(19) United States
(12) Patent Application Publication
(10) Pub. No.: US 2023/0118596 A1
(43) Pub. Date: Apr. 20, 2023

(54) METHODS FOR TREATING CANCER USING A COMBINATION OF A PD-1 ANTAGONIST, A CTLA4 ANTAGONIST, AND LENVATINIB OR A PHARMACEUTICALLY ACCPETABLE SALT THEREOF

(71) Applicants: Merck Sharp & Dohme LLC, Rahway, NJ (US); EISAI R&D Management Co., LTD., Bunkyo-ku, Tokyo (JP)

(72) Inventors: Blanca Homet Moreno, Philadelphia, PA (US); Nageatte Ibrahim, Plymouth Meeting, PA (US); Rodolfo Fleury Perini, Wynnewood, PA (US); Scott J. Diede, New Hope, PA (US); Scot W. Ebbinghaus, Lansdale, PA (US); Rachel Allison Altura, Westerly, RI (US)

(73) Assignees: Merck Sharp & Dohme LLC, Rahway, NJ (US); EISAI R&D Management Co., Ltd., Bunkyo-ku, Tokyo (JP)

(21) Appl. No.: 17/801,409

(22) PCT Filed: Mar. 4, 2021
(86) PCT No.: PCT/US2021/020858

§ 371 Date: Aug. 22, 2022

Related U.S. Application Data
(60) Provisional application No. 62/985,500, filed on Mar. 5, 2020.

(51) Int. Cl. C07K 16/28; C07D 215/233; A61P 35/00

(52) U.S. Cl. CPC C07K 16/2818; C07D 215/233; A61P 35/00

(57) ABSTRACT

Provided herein are methods of treating cancer (e.g., melanoma or RCC), which comprise administering to a human patient in need thereof: (a) a PD-1 antagonist; (b) a CTLA4 antagonist; and (c) lenvatinib represented by Formula (I), or a pharmaceutically acceptable salt thereof. Also provided are kits containing such agents and uses of therapeutic combinations of such agents for the treatment of cancer.

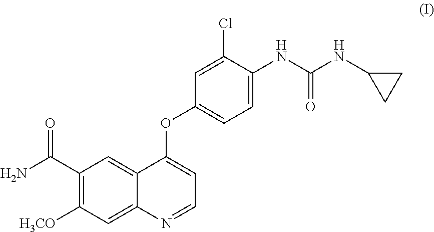 [SMILES: COc1cc2nccc(Oc3ccc(NC(=O)NC4CC4)c(Cl)c3)c2cc1C(N)=O]

### CROSS REFERENCE TO RELATED APPLICATIONS

This application claims the benefit of priority to U.S. Provisional Application No. 62/985,500 filed Mar. 5, 2020, the disclosure of which is incorporated by reference in its entirety.

### I. FIELD

Provided herein are methods for treating cancer (e.g., renal cell carcinoma (RCC), bladder cancer, breast cancer, non-small cell lung cancer (NSCLC), colorectal cancer (CRC), hepatocellular carcinoma (HCC), melanoma) using a combination of (a) a programmed death 1 protein (PD-1) antagonist, (b) an cytotoxic T-lymphocyte-associated protein 4 (CTLA4) antagonist, and (c) 4-[3-chloro-4-(cyclopropylaminocarbonyl)aminophenoxy]-7-methoxy-6-quinolinecarboxamide (lenvatinib) represented by Formula (I),

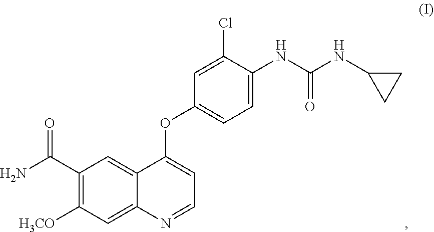 [SMILES: COc1cc2nccc(Oc3ccc(NC(=O)NC4CC4)c(Cl)c3)c2cc1C(N)=O]

or a pharmaceutically acceptable salt thereof.

### II. BACKGROUND OF THE INVENTION

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, N.J.; OPDIVO® (nivolumab), Bristol-Myers Squibb Company, Princeton, N.J.) and those that bind to the PD-L1 ligand (e.g., TECENTRIQ® (atezolizumab), Genentech, San Francisco, Calif.).

It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. One such agent that has been tested in combination with antagonists of PD-1 is an antagonist of cytotoxic T lymphocyte associated antigen 4 (abbreviated CTLA4).

CTLA4 has a very close relationship with the CD28 molecule in gene structure, chromosome location, sequence homology and gene expression. Both are receptors for the co-stimulative molecule B7, mainly expressed on the surface of activated T cells. After binding to B7, CTLA4 can inhibit the activation of mouse and human T cells, playing a negative regulating role in the activation of T cells.

CTLA4 mAbs or CTLA4 ligands can prevent CTLA4 from binding to its native ligands, thereby blocking the transduction of the T cell negative regulating signal by CTLA4 and enhancing the responsiveness of T cells to various antigens. In this aspect, results from in vivo and in vitro studies are substantially in concert. At present, there are some CTLA4 mAbs being tested in clinical trials for treating prostate cancer, bladder cancer, colorectal cancer, cancer of gastrointestinal tract, liver cancer, malignant melanoma, etc. (Grosso et al., CTLA-4 blockade in tumor models: an overview of preclinical and translational research. Cancer Immun. 13:5 (2013)).

As important factors affecting the function of T cells, CTLA4 and CTLA4 mAbs can produce specific therapeutic effects on diseases by interfering with the immune microenvironment in the body. They have high efficacy and remedy the deficiency of traditional medication, opening a novel pathway of gene therapy. CTLA4 and CTLA4 mAbs are being tested in experiments and various stages of clinical trials. For example, in autoimmune diseases, they have been shown to effectively inhibit airway hyperresponsiveness in an animal model of asthma, prevent the development of rheumatic diseases, mediate immune tolerance to an allograft in the body, and the like. On the other hand, although biological gene therapy has not shown any adverse effect in short term clinical trials, attention should be paid to the potential effect after long term application. For example, excessive blockade of CTLA4-B7 signaling by CTLA4 mAbs may result in the development of autoimmune diseases. As antibodies can specifically bind to their antigens and induce the lysis of target cells or block the progress of pathology, development and utilization of drugs based on antibodies, especially humanized antibodies have important significance in the clinical treatment of malignant tumors and other immune diseases in humans.

Tyrosine kinases are involved in the modulation of growth factor signaling and thus are an important target for cancer therapies. Lenvatinib is a multiple RTK (multi-RTK) inhibitor that selectively inhibits the kinase activities of vascular endothelial growth factor (VEGF) receptors (VEGFR1 (FLT1), VEGFR2 (KDR) and VEGFR3 (FLT4)), and fibroblast growth factor (FGF) receptors FGFR1, 2, 3 and 4 in addition to other proangiogenic and oncogenic pathway-related RTKs (including the platelet-derived growth factor (PDGF) receptor PDGFRα; KIT; and the RET proto-oncogene (RET)) involved in tumor proliferation. In particular, lenvatinib possesses a new binding mode (Type V) to VEGFR2, as confirmed through X-ray crystal structural analysis, and exhibits rapid and potent inhibition of kinase activity, according to kinetic analysis.

It has been proposed that the efficacy of anti-PD-1 or anti-PD-L1 antagonistic antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. However, there are no clear guidelines as to which agent combined with the anti-PD-1 or anti-PD-L1 antibodies may be effective or in which patients the combination may enhance the efficacy of treatment. Thus, there is an unmet need in the art for high efficacy therapeutic combinations that can generate a robust immune response to cancer.

### III. SUMMARY

The present disclosure provides methods, pharmaceutical compositions, uses and kits of treating a cancer, an infectious disease, or an infection using a combination of therapeutic agents, e.g., a combination of antibodies or antigen binding fragments thereof.

The present disclosure provides methods of treating cancer (e.g., melanoma or RCC) using a combination of a PD-1 antagonist, a CTLA4 antagonist, and 4-[3-chloro-4-(cyclopropylaminocarbonyl)aminophenoxy]-7-methoxy-6-quinolinecarboxamide (lenvatinib) represented by Formula (I),

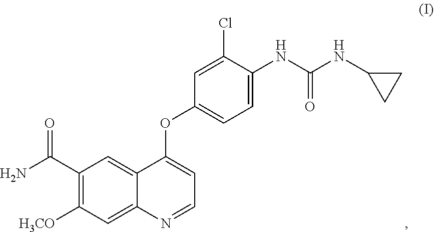 [SMILES: COc1cc2nccc(Oc3ccc(NC(=O)NC4CC4)c(Cl)c3)c2cc1C(N)=O]

or a pharmaceutically acceptable salt thereof.

The present disclosure further provides kits including a PD-1 antagonist, a CTLA4 antagonist, and 4-[3-chloro-4-(cyclopropylaminocarbonyl)aminophenoxy]-7-methoxy-6-quinolinecarboxamide (lenvatinib) represented by Formula (I),

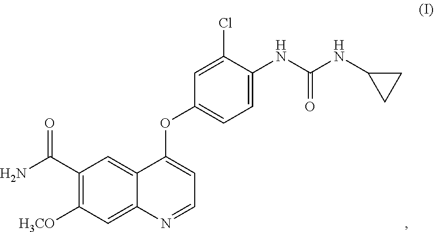 [SMILES: COc1cc2nccc(Oc3ccc(NC(=O)NC4CC4)c(Cl)c3)c2cc1C(N)=O]

or a pharmaceutically acceptable salt thereof.

Also provided herein are uses of a therapeutic combination for treating cancer (e.g., RCC, melanoma), and the therapeutic combination includes a PD-1 antagonist, a CTLA4 antagonist, and 4-[3-chloro-4-(cyclopropylaminocarbonyl)aminophenoxy]-7-methoxy-6-quinolinecarboxamide (lenvatinib) represented by Formula (I),

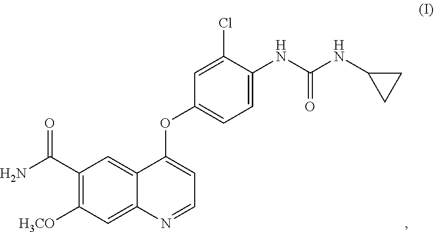 [SMILES: COc1cc2nccc(Oc3ccc(NC(=O)NC4CC4)c(Cl)c3)c2cc1C(N)=O]

a pharmaceutically acceptable salt thereof.

In one aspect, provided herein is a method of treating cancer, comprising administering to a human patient in need thereof:
(a) a PD-1 antagonist;
(b) a CTLA4 antagonist; and
(c) 4-[3-chloro-4-(cyclopropylaminocarbonyl)aminophenoxy]-7-methoxy-6-quinolinecarboxamide (lenvatinib) represented by Formula (I),

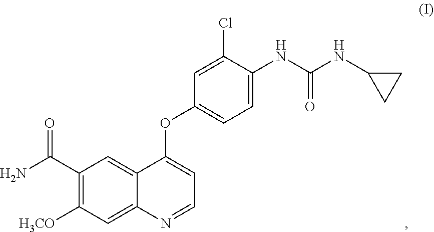 [SMILES: COc1cc2nccc(Oc3ccc(NC(=O)NC4CC4)c(Cl)c3)c2cc1C(N)=O]

or a pharmaceutically acceptable salt thereof.

In one aspect, provided herein are therapeutic combinations for use in treating cancer, comprising administering to a human patient in need thereof:
(a) a PD-1 antagonist;
(b) a CTLA4 antagonist; and
(c) 4-[3-chloro-4-(cyclopropylaminocarbonyl)aminophenoxy]-7-methoxy-6-quinolinecarboxamide (lenvatinib) represented by Formula (I),

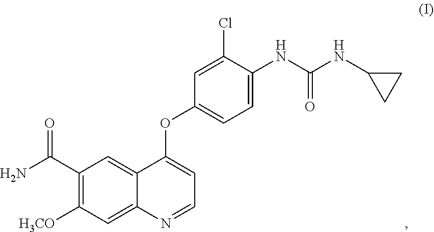 [SMILES: COc1cc2nccc(Oc3ccc(NC(=O)NC4CC4)c(Cl)c3)c2cc1C(N)=O]

or a pharmaceutically acceptable salt thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, and carcinoid cancer. For example, the cancer is a melanoma. In certain embodiments, the cancer is a treatment of PD-1 refractory melanoma. In yet another embodiment, the subject has melanoma brain metastases. In yet another embodiment, the cancer is refractory melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelofibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), and melanoma.

In certain embodiments, the cancer is metastatic. In some embodiments, the cancer is relapsed. In other embodiments, the cancer is refractory. In yet other embodiments, the cancer is relapsed and refractory. In certain embodiments, the cancer is resectable.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is melanoma. In yet another embodiment, the cancer is melanoma and the patient has melanoma brain metastases. In yet another embodiment, the cancer is refractory melanoma. In one embodiment, the cancer is PD-1 refractory melanoma. In one embodiment, the cancer is advanced melanoma. In another embodiment, the cancer is metastatic melanoma. In yet another embodiment, the cancer is relapsed melanoma. In yet still another embodiment, the cancer is relapsed and refractory melanoma. In various embodiments, the cancer is locally advanced or resectable. In various embodiments, the cancer has been treated with an adjuvant. In various embodiments, the cancer has been treated with a neoadjuvant.

In one embodiment, the cancer is advanced renal cell carcinoma (RCC). In another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In another aspect, provided herein is a kit comprising:
(a) a PD-1 antagonist;
(b) a CTLA4 antagonist; and
(c) 4-[3-chloro-4-(cyclopropylaminocarbonyl)aminophenoxy]-7-methoxy-6-quinolinecarboxamide (lenvatinib) represented by Formula (I),

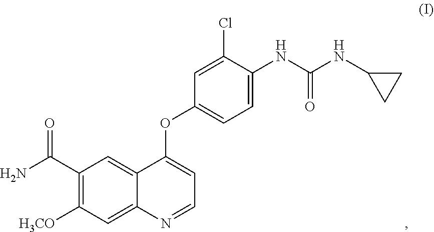 [SMILES: COc1cc2nccc(Oc3ccc(NC(=O)NC4CC4)c(Cl)c3)c2cc1C(N)=O]

or a pharmaceutically acceptable salt thereof.

In certain embodiments, the kit further comprises instructions for administering to a human patient the PD-1 antagonist, the CTLA4 antagonist, and 4-[3-chloro-4-(cyclopropylaminocarbonyl)aminophenoxy]-7-methoxy-6-quinolinecarboxamide (lenvatinib) represented by Formula (I),

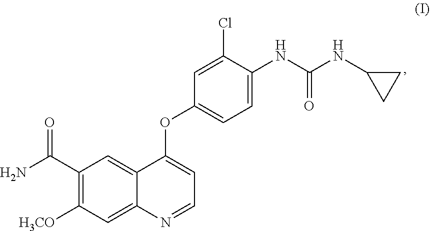 [SMILES: COc1cc2nccc(Oc3ccc(NC(=O)NC4CC4)c(Cl)c3)c2cc1C(N)=O]

or a pharmaceutically acceptable salt thereof.

In still another aspect, provided herein is use of a therapeutic combination for treating cancer in a human patient, wherein the therapeutic combination comprises:
(a) a PD-1 antagonist;
(b) a CTLA4 antagonist; and
(c) 4-[3-chloro-4-(cyclopropylaminocarbonyl)aminophenoxy]-7-methoxy-6-quinolinecarboxamide (lenvatinib) represented by Formula (I),

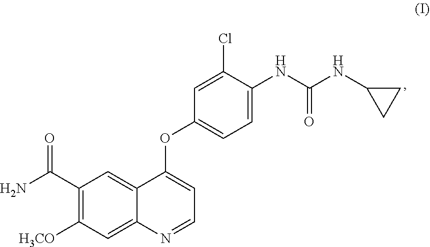 [SMILES: COc1cc2nccc(Oc3ccc(NC(=O)NC4CC4)c(Cl)c3)c2cc1C(N)=O]

or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is melanoma. In yet another embodiment, the cancer is melanoma and the patient has melanoma brain metastases. In yet another embodiment, the cancer is refractory melanoma.

In one embodiment, the cancer is advanced RCC. In another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In various embodiments, the subject or patient has a cancer and expresses at least one Breast Cancer gene (e.g., BRCA). In various embodiments, the cancer or a sample from the subject is found to have a level or to express at least one Breast Cancer gene (BRCA). In various embodiments, the at least one BRCA gene is BRCA1 or BRCA2. In an embodiment, the cancer is BRCA negative. For example, the cancer (for example breast cancer and ovarian cancer) is a BRCA negative cancer. In an embodiment, the cancer is BRCA positive.

In certain embodiments of various methods, pharmaceutical compositions, kits, uses, or the combinations for use provided herein, the subject is a human patient.

In certain embodiments the methods, pharmaceutical compositions, kits, uses, or the combinations for use provided herein are for treating cancer.

In certain embodiments of various methods, pharmaceutical compositions, kits, or uses provided herein, the PD-1 antagonist is an anti-human PD-1 monoclonal antibody or antigen binding fragment thereof.

In other embodiments of various methods, pharmaceutical compositions, kits, or uses provided herein, the PD-1 antagonist is an anti-human PD-L1 monoclonal antibody or antigen binding fragment thereof.

In some embodiments of various methods, pharmaceutical compositions, kits, or uses provided herein, the anti-human PD-1 monoclonal antibody is a humanized antibody.

In other embodiments of various methods, pharmaceutical compositions, kits, or uses provided herein, the anti-human PD-1 monoclonal antibody is a human antibody.

In some embodiments of various methods, pharmaceutical compositions, kits, or uses provided herein, the anti-human PD-L1 monoclonal antibody is a humanized antibody.

In other embodiments of various methods, pharmaceutical compositions, kits, or uses provided herein, the anti-human PD-L1 monoclonal antibody is a human antibody.

In certain embodiments of various methods, pharmaceutical compositions, kits, or uses provided herein, the CTLA4 antagonist is an anti-human CTLA4 monoclonal antibody or antigen binding fragment thereof.

In some embodiments of various methods, pharmaceutical compositions, kits, or uses provided herein, the anti-human CTLA4 monoclonal antibody is a humanized antibody.

In other embodiments of various methods, pharmaceutical compositions, kits, or uses provided herein, the anti-human CTLA4 monoclonal antibody is a human antibody.

In still other embodiments of various methods, pharmaceutical compositions, kits, uses provided herein, the anti-PD-1 antibody is independently selected from pembrolizumab, nivolumab, cemiplimab, sintilimab, tislelizumab, camrelizumab and toripalimab.

In one embodiment of various methods, pharmaceutical compositions, kits, or uses provided herein, the anti-human PD-1 monoclonal antibody is pembrolizumab.

In another embodiment of various methods, pharmaceutical compositions, kits, or uses provided herein, the anti-human PD-1 monoclonal antibody is nivolumab.

In another embodiment of various methods, pharmaceutical compositions, kits, or uses provided herein, the anti-human PD-1 monoclonal antibody is cemiplimab.

In yet another embodiment of various methods, pharmaceutical compositions, kits, or uses provided herein, the anti-human PD-1 monoclonal antibody or antigen binding fragment thereof is pidilizumab (U.S. Pat. No. 7,332,582).

In yet another embodiment of various methods, pharmaceutical compositions, kits, or uses provided herein, the anti-human PD-1 monoclonal antibody or antigen binding fragment thereof is AMP-514 (MedImmune LLC, Gaithersburg, Md.).

In yet another embodiment of various methods, pharmaceutical compositions, kits, or uses eprovided herein, the anti-human PD-1 monoclonal antibody or antigen binding fragment thereof is PDR001 (U.S. Pat. No. 9,683,048).

In yet another embodiment of various methods, pharmaceutical compositions, kits, or uses provided herein, the anti-human PD-1 monoclonal antibody or antigen binding fragment thereof is BGB-A317 (U.S. Pat. No. 8,735,553).

In yet another embodiment of various methods, pharmaceutical compositions, kits, or uses provided herein, the anti-human PD-1 monoclonal antibody or antigen binding fragment thereof is MGA012 (MacroGenics, Rockville, Md.).

In certain embodiments of various methods, kits, or uses provided herein, the anti-human CTLA4 monoclonal antibody comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

In some embodiments of various methods, kits, or uses provided herein, the anti-human CTLA4 monoclonal antibody comprises a V_{L} region comprising an amino acid sequence as set forth in SEQ ID NO:14, and a V_{H} region comprising an amino acid sequence as set forth in SEQ ID NO:15.

In other embodiments of various methods, kits, or uses provided herein, the anti-human CTLA4 monoclonal antibody comprises a light chain comprising or consisting of an amino acid sequence as set forth in SEQ ID NO:23 and a heavy chain comprising or consisting of an amino acid sequence as set forth in SEQ ID NO:22.

In yet still another embodiment of various methods, kits, or uses provided herein, the lenvatinib or a pharmaceutically acceptable salt thereof is lenvatinib mesylate. Capsules for oral administration contain 4 mg or 10 mg of lenvatinib, equivalent to 4.90 mg or 12.25 mg of lenvatinib mesylate, respectively. In another embodiment, when a pharmaceutically acceptable salt of lenvatinib is administered, such as lenvatinib mesylate, and the dose of lenvatinib to be used is 4 mg, a medical practitioner would know to administer 4.90 mg of lenvatinib mesylate. In another embodiment, when a pharmaceutically acceptable salt of lenvatinib is administered, such as lenvatinib mesylate, and the dose of lenvatinib to be used is 10 mg, a medical practitioner would know to administer 12.25 mg of lenvatinib mesylate.

In one specific embodiment of various methods, kits, or uses provided herein, the PD-1 antagonist is pembrolizumab; and the CTLA4 antagonist is a monoclonal antibody or antigen binding fragment thereof comprising a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

In one specific embodiment of various methods, kits, or uses provided herein, the PD-1 antagonist is nivolumab; and the CTLA4 antagonist is a monoclonal antibody or antigen binding fragment thereof comprising a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

In one specific embodiment of various methods, kits, or uses provided herein, the PD-1 antagonist is cemiplimab; and the CTLA4 antagonist is a monoclonal antibody or antigen binding fragment thereof comprising a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

In one specific embodiment of various methods, kits, or uses provided herein, the CTLA4 antagonist comprises six complimentary determining regions (CDRs) wherein the six CDRs comprise (a) CDR1, CDR2, and CDR3 of the light chain (LC) variable domain having the amino acid sequence shown in SEQ ID NO: 14; and (b) CDR1, CDR2, and CDR3 of the light chain (LC) variable domain having the amino acid sequence shown in SEQ ID NO: 15.

In another aspect, provided herein is a method of enhancing T cell activity, comprising contacting the T cells with:
(a) an anti-human PD-1 antibody (e.g., monoclonal antibody) or antigen binding fragment thereof;
(b) an anti-human CTLA4 antibody (e.g., monoclonal antibody) or antigen binding fragment thereof, and
(a) 4-[3-chloro-4-(cyclopropylaminocarbonyl)aminophenoxy]-7-methoxy-6-quinolinecarboxamide (lenvatinib) represented by Formula (I),

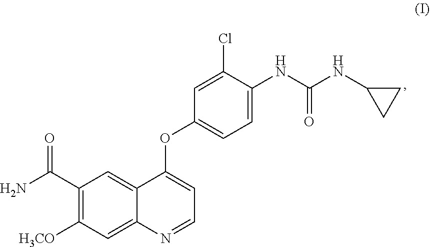 [SMILES: COc1cc2nccc(Oc3ccc(NC(=O)NC4CC4)c(Cl)c3)c2cc1C(N)=O]

or a pharmaceutically acceptable salt thereof.

In some embodiments, the enhancement of T cell activity occurs in vitro. In other embodiments, the enhancement of T cell activity occurs in vivo. For example, the enhancement is in a subject including but not limited to a human subject or human patient.

In certain embodiments, the enhancement of T cell activity is measured by increased cytokine production. In other embodiments, the enhancement of T cell activity is measured by increased cell proliferation.

In some embodiments, provided herein is a method of increasing cytokine production of T cells, comprising contacting the T cells with:
(a) an anti-human PD-1 antibody (e.g., monoclonal antibody) or antigen binding fragment thereof;
(b) an anti-human CTLA4 antibody (e.g., monoclonal antibody) or antigen binding fragment thereof, and
(a) 4-[3-chloro-4-(cyclopropylaminocarbonyl)aminophenoxy]-7-methoxy-6-quinolinecarboxamide (lenvatinib) represented by Formula (I),

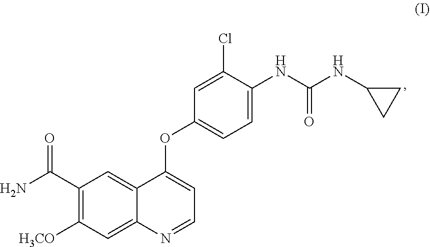 [SMILES: COc1cc2nccc(Oc3ccc(NC(=O)NC4CC4)c(Cl)c3)c2cc1C(N)=O]

or a pharmaceutically acceptable salt thereof.

In some embodiments, the increased cytokine production of T cells occurs in vitro. In other embodiments, the increased cytokine production of T cells occurs in vivo.

In some embodiments of various methods described herein, the human patient is administered 200 mg, 240 mg, or 2 mg/kg pembrolizumab, and pembrolizumab is administered once every three weeks. In one embodiment, the human patient is administered 200 mg pembrolizumab once every three weeks. In one embodiment, the human patient is administered 240 mg pembrolizumab once every three weeks. In one embodiment, the human patient is administered 2 mg/kg pembrolizumab once every three weeks.

In certain embodiments of various methods described herein, the human patient is administered 400 mg pembrolizumab, and pembrolizumab is administered once every six weeks.

In other embodiments of various methods described herein, the human patient is administered 240 mg or 3 mg/kg nivolumab once every two weeks, or 480 mg nivolumab once every four weeks. In one specific embodiment, the human patient is administered 240 mg nivolumab once every two weeks. In one specific embodiment, the human patient is administered 3 mg/kg nivolumab once every two weeks. In one specific embodiment, the human patient is administered 480 mg nivolumab once every four weeks.

In yet other embodiments of various methods described herein, the human patient is administered 350 mg cemiplimab, and cemiplimab is administered once every three weeks.

In still other embodiments of various methods described herein, the anti-human CTLA4 monoclonal antibody or antigen binding fragment thereof comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively, the human patient is administered from about 1 mg to about 100 mg of the anti-human CTLA4 antibody once every three weeks. In some embodiments, the human patient is administered 1, 10, 25, 50, 75, or 100 mg of the anti-human CTLA4 antibody once every three weeks. In one specific embodiment, the human patient is administered 1 mg of the anti-human CTLA4 antibody once every three weeks. In one specific embodiment, the human patient is administered 10 mg of the anti-human CTLA4 antibody once every three weeks. In one specific embodiment, the human patient is administered 25 mg of the anti-human CTLA4 antibody once every three weeks. In one specific embodiment, the human patient is administered 50 mg of the anti-human CTLA4 antibody once every three weeks. In one specific embodiment, the human patient is administered 75 mg of the anti-human CTLA4 antibody once every three weeks. In one specific embodiment, the human patient is administered 100 mg of the anti-human CTLA4 antibody once every three weeks.

In still other embodiments of various methods described herein, the anti-human CTLA4 monoclonal antibody or antigen binding fragment thereof comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively, the human patient is administered from about 1 mg to about 100 mg of the anti-human CTLA4 antibody once every six weeks. In some embodiments, the human patient is administered 1, 10, 25, 50, 75, or 100 mg of the anti-human CTLA4 antibody once every six weeks. In one specific embodiment, the human patient is administered 1 mg of the anti-human CTLA4 antibody once every six weeks. In one specific embodiment, the human patient is administered 10 mg of the anti-human CTLA4 antibody once every six weeks. In one specific embodiment, the human patient is administered 25 mg of the anti-human CTLA4 antibody once every six weeks. In one specific embodiment, the human patient is administered 50 mg of the anti-human CTLA4 antibody once every six weeks. In one specific embodiment, the human patient is administered 75 mg of the anti-human CTLA4 antibody once every six weeks. In one specific embodiment, the human patient is administered 100 mg of the anti-human CTLA4 antibody once every six weeks.

In yet still other embodiments of various methods described herein, the human patient is administered 8, 10, 12, 14, 18, 20, or 24 mg lenvatinib once daily.

Thus, in some embodiments, the human patient is administered:
(a) 200 mg, 240 mg, or 2 mg/kg pembrolizumab once every three or six weeks;
(b) 1, 10, 25, 50, 75, or 100 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 8, 10, 12, 14, 18, 20, or 24 mg lenvatinib once daily.

In certain embodiments, the human patient is administered:
(a) 200 mg, 240 mg, or 2 mg/kg pembrolizumab once every six weeks;
(b) 1, 10, 25, 50, 75, or 100 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 8, 10, 12, 14, 18, 20, or 24 mg lenvatinib once daily.

In certain embodiments, the human patient is administered:
(a) 200 mg pembrolizumab once every six weeks;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 20 mg lenvatinib once daily.

In certain embodiments, the human patient is administered:
(a) 240 mg pembrolizumab once every six weeks;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 20 mg lenvatinib once daily.

In certain embodiments, the human patient is administered:
(a) 2 mg/kg pembrolizumab once every six weeks;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 20 mg lenvatinib once daily.

In certain embodiments, the human patient is administered:
(a) 400 mg pembrolizumab once every six weeks;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 20 mg lenvatinib once daily.

In a specific embodiment, provided herein is a method of treating RCC, comprising administering to a human patient in need thereof:
(a) 400 mg pembrolizumab once every six weeks;
(b) 25 mg of an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 20 mg lenvatinib once daily.

In a specific embodiment, provided herein is a method of treating melanoma, comprising administering to a human patient in need thereof:
(a) 400 mg pembrolizumab once every six weeks;
(b) 25 mg of an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively once every six weeks; and
(c) 20 mg lenvatinib once daily.

In certain embodiments of such a method, the anti-human PD-1 monoclonal antibody and the anti-human CTLA4 monoclonal antibody are administered on the same day. In some embodiments, the anti-human PD-1 monoclonal antibody and the anti-human CTLA4 monoclonal antibody are administered sequentially. In other embodiments, the anti-human PD-1 monoclonal antibody and the anti-human CTLA4 monoclonal antibody are administered concurrently.

In some embodiments of various methods, kits, or uses described herein, the pharmaceutically acceptable salt of lenvatinib—lenvatinib mesylate—can be used. When lenvatinib mesylate is used, the dosage of lenvatinib mesylate is appropriately adjusted to provide the same molar equivalents of lenvatinib as 8, 10, 12, 14, 18, 20, or 24 mg lenvatinib provides.

### IV. DETAILED DESCRIPTION OF THE INVENTION

### 1. Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-PD-1 antibody or antigen binding fragment thereof, an anti-CTLA4 antibody or antigen binding fragment thereof, or lenvatinib, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, an anti-CTLA4 antibody, and lenvatinib as described herein) into a patient, such as by oral, mucosal, intradermal, intravenous, subcutaneous, intramuscular delivery, and/or any other methods of physical delivery described herein or known in the art.

“Anti-PD-1 antibody” means a monoclonal antibody that specifically binds to human PD-1. PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (Sharpe, A. H, Wherry, E. J., Ahmed R., and Freeman G. J. The function of programmed cell death 1 and its ligands in regulating autoimmunity and infection. Nature Immunology (2007); 8:239-245). Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong H et al. Nat Med. 2002 August; 8(8):793-800; Yang et al. Invest Ophthalmol Vis Sci. 2008 June; 49 (6 (2008): 49: 2518-2525; Ghebeh et al. Neoplasia (2006) 8: 190-198; Hamanishi J et al. Proceeding of the National Academy of Sciences (2007): 104: 3360-3365; Thompson R H et al. Clinical genitourin Cancer (2006): 5: 206-211; Nomi, T. Sho, M., Akahori, T., et al. Clinical Cancer Research (2007); 13:2151-2157; Ohigashi Y et al. Clin. Cancer Research (2005): 11: 2947-2953; Inman et al. Cancer (2007): 109: 1499-1505; Shimauchi T et al. Int. J. Cancer (2007): 121:2585-2590; Gao et al. Clinical Cancer Research (2009) 15: 971-979; Nakanishi J. Cancer Immunol Immunother. (2007) 56: 1173-1182; Hino et al. Cancer (2010): 00: 1-9). Similarly, PD-1 expression on tumor infiltrating lymphocytes was found to mark dysfunctional T cells in breast cancer and melanoma (Ghebeh H., BMC Cancer. 2008 Feb. 23; 8:57; and Ahmadzadeh M et al. Blood (2009) 114: 1537-1544) and to correlate with poor prognosis in renal cancer (Thompson R H et al, Clinical Cancer Research (2007) 15: 1757-1761). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) mAb with high specificity of binding to the programmed cell death 1 (PD 1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications. “PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. The PD-1 antagonist is not the anti-PD-L1 monoclonal antibody atezolizumab.

An “CTLA4 antagonist” means any chemical compound or biological molecule that specifically binds to human CTLA4 and blocks the interaction of CTLA4 with its ligands, CD80 (B7.1) and CD 86 (B7.2). An “anti-CTLA4 antibody” means a monoclonal antibody that specifically binds to human CTLA4. The anti-CTLA4 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}”.

Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} f-sheet framework, or one of three hypervariable regions (L1, L2, or L3) within the non-framework region of the antibody V_{L} f-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDRI | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDRI | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1 or CTLA4 is an antibody that exhibits preferential binding to PD-1 or CTLA4 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1 or CTLA4). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorph (e.g., rabbits), rodent (e.g., rats or mice), Procyon lotor (e.g., raccoons). In particular embodiments, the subject is human.

The term “subject in need thereof” as used herein refers to a subject diagnosed with or suspected of having cancer or an infectious disease as defined herein.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody or an anti-CTLA4 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant or an anti-CTLA4 antibody variant) at least retains the antibody functional activity. In specific embodiments, an anti-PD-1 antibody variant binds to PD-1 and/or is antagonistic to PD-1 activity. In some embodiments, an anti-CTLA4 antibody variant binds to CTLA4 and/or is antagonistic to CTLA4 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared × 100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Treat” or “treating” cancer as used herein means to administer a therapeutic combination of an anti-human PD-1 monoclonal antibody or antigen binding fragment thereof, an anti-human CTLA4 monoclonal antibody or antigen binding fragment thereof, and lenvatinib or a pharmaceutically acceptable salt thereof, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth, comprising administration by oral, mucosal, intradermal, intravenous, subcutaneous, intramuscular delivery, and/or any other methods of physical delivery described herein or known in the art. “Treatment” may include one or more of the following: inducing/increasing an antitumor immune response, decreasing the number of one or more tumor markers, halting or delaying the growth of a tumor or blood cancer or progression of disease such as cancer, stabilization of disease, inhibiting the growth or survival of tumor cells, eliminating or reducing the size of one or more cancerous lesions or tumors, decreasing the level of one or more tumor markers, ameliorating or abrogating the clinical manifestations of disease, reducing the severity or duration of the clinical symptoms, prolonging the survival or patient relative to the expected survival in a similar untreated patient, and inducing complete or partial remission of a cancerous condition, wherein the disease is cancer, and wherein the cancer is selected from the group consisting of bladder cancer, breast cancer, non-small cell lung cancer (NSCLC), colorectal cancer (CRC), renal cell carcinoma (RCC), hepatocellular carcinoma (HCC), and melanoma. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control × 100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “pharmaceutically acceptable carrier” refers to any inactive substance that is suitable for use in a formulation for the delivery of a therapeutic agent. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

As used herein, the terms “combination,” “combination therapy,” and “therapeutic combination” refer to treatments in which at least one anti-human PD-1 monoclonal antibody or antigen-binding fragment thereof, at least one anti-human CTLA4 monoclonal antibody or antigen-binding fragment thereof, and lenvatinib or a pharmaceutically acceptable salt thereof, and optionally additional therapeutic agents, each are administered to a patient in a coordinated manner, over an overlapping period of time. The period of treatment with the at least one anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the at least one anti-human CTLA4 monoclonal antibody (or antigen-binding fragment thereof) (the “anti-CTLA4 treatment”) is the period of time that a patient undergoes treatment with the anti-human CTLA4 monoclonal antibody (or antigen-binding fragment thereof); that is, the period of time from the initial dosing with the anti-human CTLA4 monoclonal antibody (or antigen-binding fragment thereof) through the final day of a treatment cycle. The period of treatment with lenvatinib or a pharmaceutically acceptable salt thereof (the “lenvatinib treatment”) is the period of time that a patient undergoes treatment with lenvatinib; that is, the period of time from the initial dosing with lenvatinib through the final day of a treatment cycle. In the methods and therapeutic combinations described herein, the anti-PD-1 treatment overlaps by at least one day with the anti-CTLA4 treatment and overlaps by at least one day with the lenvatinib treatment. In certain embodiments, the anti-PD-1 treatment, the anti-CTLA4 treatment, and the lenvatinib treatment are the same period of time. In some embodiments, the anti-PD-1 treatment begins prior to the anti-CTLA4 and/or the lenvatinib treatment. In other embodiments, the anti-PD-1 treatment begins after the anti-CTLA4 and/or the lenvatinib treatment. In yet other embodiments, the anti-CTLA4 treatment begins prior to the anti-PD-1 and/or the lenvatinib treatment. In still other embodiments, the anti-CTLA4 treatment begins after the anti-PD-1 and/or the lenvatinib treatment. In some embodiments, the lenvatinib treatment begins prior to the anti-CTLA4 and/or the anti-PD-1 treatment. In other embodiments, the lenvatinib treatment begins after the anti-CTLA4 and/or the anti-PD-1 treatment. In certain embodiments, the anti-PD-1 treatment is terminated prior to termination of the anti-CTLA4 and/or the lenvatinib treatment. In other embodiments, the anti-PD-1 treatment is terminated after termination of the anti-CTLA4 and/or the lenvatinib treatment. In yet other embodiments, the anti-CTLA4 treatment is terminated prior to termination of the anti-PD-1 and/or the lenvatinib treatment. In still other embodiments, the anti-CTLA4 treatment is terminated after termination of the anti-PD-1 and/or the lenvatinib treatment. In certain embodiments, the lenvatinib treatment is terminated prior to termination of the anti-CTLA4 and/or the anti-PD-1 treatment. In other embodiments, the lenvatinib treatment is terminated after termination of the anti-CTLA4 and/or the anti-PD-1 treatment.

The terms “treatment regimen,” “dosing protocol,” and “dosing regimen” are used interchangeably to refer to the dose and timing of administration of each therapeutic agent in a combination therapy of the disclosure.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include, but are not limited to, squamous cell carcinoma, myeloma, small-cell lung cancer, non-small cell lung cancer, glioma, Hodgkin lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

As used herein, the term “effective amount” refer to an amount of an anti-PD-1 antibody or antigen binding fragment, an anti-CTLA4 antibody or antigen binding fragment of the invention, and lenvatinib that, when administered alone or in combination with an additional therapeutic agent to a cell, tissue, or subject, is effective to cause a measurable improvement in one or more symptoms of an infection or a disease, for example cancer or the progression of cancer. An effective dose further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic may result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. Toxicity and therapeutic efficacy of the antibodies or antigen binding fragments of the invention, administered alone or in combination with another therapeutic agent, can be determined by any number of systems or means. For example, the toxicity and therapeutic efficacy of the antibodies or antigen binding fragments of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

It is understood that wherever embodiments are described herein with the language “comprising,” otherwise analogous embodiments described in terms of “consisting of” and/or “consisting essentially of” are also provided.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### 2. Abbreviations with Expanded Term

TABLE 3
Abbreviations

| Abbreviation | Expanded Term |
|---|---|
| 1L | First line |
| ADA | anti drug antibody |
| AE | adverse event |
| AJCC | American Joint Committee on Cancer |
| ALT | Alanine aminotransferase |
| anti-CTLA-4 | anti cytotoxic T-lymphocyte-associated protein 4 |
| APaT | All-Participants-as-Treated |
| aPTT | Activated partial thromboplastin time |
| BCG | Bacillus Calmette-Guérin |
| BICR | blinded independent central review |
| BP | blood pressure |
| BRAF | v-raf murine sarcoma viral oncogene homolog B1 |
| C1 | Cycle 1 |
| C2 | Cycle 2 |
| C3 | Cycle 3 |
| C4 | Cycle 4 |
| C5 | Cycle 5 |
| C6 | Cycle 6 |
| C10 | Cycle 10 |
| C14 | Cycle 14 |
| C1D1 | Cycle 1 Day 1 |
| CD | cluster of differentiation |
| CDR | Complementary determining region |
| CD28 | cluster of differentiation 28 |
| CHO | Chinese hamster ovary |
| CIV | central imaging vendor |
| CNS | central nervous system |
| CR | complete response |
| CSR | Clinical Study Report |
| CT | computed tomography |
| CTCAE | Common Terminology Criteria for Adverse Events |
| CTCAE 5.0 | Common Terminology Criteria for Adverse Events, |
| | version 5.0 |
| CTLA-4 | cytotoxic T-lymphocyte-associated protein 4 |
| D15 | Day 1 |
| D8 | Day 8 |
| D15 | Day 15 |
| D/C | discontinue/discontinuing |
| DLT | dose-limiting toxicity |
| DNA | deoxyribonucleic acid |
| DOR | Duration of Response |
| ECG | Electrocardiogram |
| ECHO | Echocardiogram |
| ECI | event of clinical interest |
| ECOG | Eastern Cooperative Oncology Group |
| EFS | event-free survival |
| EOT | End of treatment |
| EU | European Union |
| FR | framework region |
| HBsAg | Hepatitis B surface antigen |
| HBV | Hepatitis B virus |
| HCV | Hepatitis C virus |
| HIV | human immunodeficiency virus |
| IA | interim analysis |
| IB | Investigator's Brochure |
| IC50 | mean inhibitory concentration |
| ICF | Informed Consent Form |
| iCPD | iRECIST confirmed progressive disease |
| iCR | iRECIST complete response |
| IgG | Immunoglobulin G |
| IgG1 | Immunoglobulin G, subclass 1 |
| IMP | Investigational medicinal product |
| IND | Investigational New Drug |
| IO | immuno-oncology |
| IP | Intraperitoneal |
| iPR | iRECIST partial response |
| irAE | immune-related adverse event |
| iRECIST | Response Evaluation Criteria in Solid Tumors 1.1 |
| | for immune-based therapeutics |
| iSD | iRECIST stable disease |
| IT | Intratumoral |
| ITT | immunoglobulin tail tyrosine |
| ITIM | immunoreceptor tyrosine-based inhibitory motif |
| iUPD | iRECIST unconfirmed progressive disease |
| IV | Intravenous |
| IVD | in vitro diagnostic |
| kg | Kilogram |
| LVEF | left ventricular ejection fraction |
| mAb | monoclonal antibody |
| MedDRA | Medical Dictionary for Regulatory Activities |
| Mg | Milligram |
| mL | Milliliter |
| Mm | Millimeter |
| mm³ | cubic millimeter, 0.001 mL |
| MPK | milligram per kilogram |
| MRI | magnetic resonance imaging |
| MSD | Merck Sharp and Dohme Corp., a subsidiary of |
| | Merck & Co., Inc. |
| MSI | microsatellite instability |
| mTPI | modified Toxicity Probability Interval |
| MUGA | multiple-gated acquisition |
| N | number of subjects in a treatment group |
| NCI | National Cancer Institute |
| NE | not estimable |
| NK | Natural killer (cells) |
| NK T | Natural killer T (cells) |
| NSCLC | non-small cell lung cancer |
| OR | overall response |
| ORR | objective response rate |
| OS | overall survival |
| PBPK | physiologically-based PK |
| PD-1 | programmed cell-death 1 |
| PDGFR | platelet-derived growth factor receptor |
| PD-L1 | programmed cell-death ligand 1 |
| PD-L2 | programmed cell-death ligand 2 |
| PE | Physical examination |
| PET | positron emission tomography |
| PFS | Progression-free survival |
| PK | Pharmacokinetic |
| PO | by mouth (orally) |
| PP | Per Protocol |
| pPR | pathological partial response |
| PR | partial response |
| PRO | patient-reported outcome |
| pRR | pathological response rate |
| PT | prothrombin time |
| PTT | partial thromboplastin time |
| Q2W | every 2 weeks |
| Q3W | every 3 weeks |
| Q6W | every 6 weeks |
| Q9W | every 9 weeks |
| Q12W | every 12 weeks |
| Q24W | every 24 weeks |
| QD | once daily/one dose per day |
| Q5D | dose every 5 days |
| Q7D | dose every 7 days |
| Q10D | dose every 10 days |
| QoL | quality of life |
| QTc | The QTc interval is the length of time it |
| | take the electrical system in the heart to |
| | repolarize, adjusted for heart rate. |
| RCC | renal cell carcinoma |
| RECIST | Response Evaluation Criteria in Solid Tumors |
| RECIST 1.1 | Response Evaluation Criteria in Solid Tumors 1.1 |
| RENCA | Renal cell carcinoma |
| RNA | ribonucleic acid |
| RP2D | recommended Phase 2 dose |
| RR | Respiratory rate |
| SAE | serious adverse event |
| SD | stable disease |
| SEM | standard error of the mean |
| SoA | Schedule of Activities |
| T3 | Triiodothyronine |
| T/C | median tumor volume of the treated animal/ |
| | Median tumor volume of the control animal |
| TSH | thyroid-stimulating hormone |
| μg, μg, UG | Microgram |
| US | United States |
| VEGF | vascular endothelial growth factor |
| VEGFR | vascular endothelial growth factor receptors |
| V_{H} | immunoglobulin heavy chain variable region |
| V_{K} | immunoglobulin kappa light chain variable region |
| V_{L} | immunoglobulin light chain variable region |
| WOCBP | woman/women of childbearing potential |

### 3. PD-1 Antagonist

Provided herein are PD-1 antagonists or anti-human PD-1 monoclonal antibodies that can be used in the various methods, kits, and uses disclosed herein, including any chemical compound or biological molecule that blocks binding of PD-L1 to PD-1 and preferably also blocks binding of PD-L2 to PD-1.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, pidilizumab (U.S. Pat. No. 7,332,582), AMP-514 (MedImmune LLC, Gaithersburg, Md.), PDR001 (U.S. Pat. No. 9,683,048), BGB-A317 (U.S. Pat. No. 8,735,553), and MGA012 (MacroGenics, Rockville, Md.). In one embodiment, the anti-human PD-1 monoclonal antibody is pembrolizumab. In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In certain embodiments of various methods, pharmaceutical compositions, kits, or uses provided herein, the anti-human PD-1 monoclonal antibody or antigen binding fragment thereof comprises a light chain variable region (V_{L}) complementarity determining region 1 (CDR1), a V_{L} CDR2, and a V_{L} CDR3 comprising amino acid sequences as set forth in SEQ ID NOs: 34, 35, and 36, respectively, and a heavy chain variable region (V_{H}) CDR1, a V_{H} CDR2, and a V_{H} CDR3 comprising amino acid sequences as set forth in SEQ ID NOs:39, 40, and 41, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:37, and a V_{H} region comprising an amino acid sequence as set forth in SEQ ID NO:42.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:38 and a heavy chain comprising or consisting of an amino acid sequence as set forth in SEQ ID NO:43.

In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

Examples of monoclonal antibodies that bind to human PD-L1 that can be used in various methods, kits, and uses described herein are disclosed in U.S. Pat. No. 8,383,796, the disclosures of which are incorporated by reference herein in their entireties. Specific anti-human PD-L1 monoclonal antibodies useful as the PD-1 antagonist in the various methods, kits, and uses described include durvalumab, avelumab, and BMS-936559.

Other PD-1 antagonists useful in various methods, kits, and uses described herein include an immunoadhesion molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In various embodiments, the anti-human PD-1 or anti-human PD-L1 monoclonal antibody or antigen binding fragment thereof comprises a variant of the amino acid sequences of the anti-human PD-1 or anti-human PD-L1 antibodies described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein and a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1.

In one embodiment, the anti-human PD-1 or anti-human PD-L1 monoclonal antibody or antigen binding fragment thereof has a V_{L} domain having up to 1, 2, 3, 4, 5 or more amino acid substitutions, deletions and/or additions in one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain having up to 1, 2, 3, 4, 5 or more amino acid substitutions, deletions, and/or additions in one of the V_{L} domains of the anti-human PD-1 or anti-human PD-L1 antibodies described herein and a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1.

In various embodiments, the anti-human PD-1 or anti-human PD-L1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-human PD-1 or anti-human PD-L1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

In some embodiments, the anti-human PD-1 or anti-human PD-L1 monoclonal antibody is a human antibody. In other embodiments, the anti-human PD-1 or anti-human PD-L1 monoclonal antibody is a humanized antibody.

In some embodiments, the light chain of the anti-human PD-1 or anti-human PD-L1 monoclonal antibody has a human kappa backbone. In other embodiments, the light chain of the anti-human PD-1 or anti-human PD-L1 monoclonal antibody has a human lambda backbone.

In some embodiments, the heavy chain of the anti-human PD-1 or anti-human PD-L1 monoclonal antibody has a human IgG1 backbone. In other embodiments, the heavy chain of the anti-human PD-1 or anti-human PD-L1 monoclonal antibody has a human IgG2 backbone. In yet other embodiments, the heavy chain of the anti-human PD-1 or anti-human PD-L1 monoclonal antibody has a human IgG3 backbone. In still other embodiments, the heavy chain of the anti-human PD-1 or anti-human PD-L1 monoclonal antibody has a human IgG4 backbone.

In some embodiments, the heavy chain of the anti-human PD-1 or anti-human PD-L1 monoclonal antibody has a human IgG1 variant backbone. In other embodiments, the heavy chain of the anti-human PD-1 or anti-human PD-L1 monoclonal antibody has a human IgG2 variant backbone. In yet other embodiments, the heavy chain of the anti-human PD-1 or anti-human PD-L1 monoclonal antibody has a human IgG3 variant backbone. In still other embodiments, the heavy chain of the anti-human PD-1 or anti-human PD-L1 monoclonal antibody has a human IgG4 variant (e.g., IgG4 with S228P mutation) backbone.

### 4. CTLA4 Antagonists

An “anti-CTLA4 antibody” useful in any of the treatment methods, compositions, kits, and uses of the present invention include monoclonal antibodies (mAb), or antigen binding fragments thereof, which specifically bind to human CTLA4 and block the interaction of CTLA4 with its ligands, CD80 (B7.1) and CD 86 (B7.2). An anti-CTLA4 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

In one embodiment of the treatment methods, compositions, kits and uses of the invention, the anti-CTLA4 antibody is the human monoclonal antibody 10D1, now known as ipilimumab, and marketed as Yervoy™, which is disclosed in U.S. Pat. No. 6,984,720 and WHO Drug Information 19(4): 61 (2005). In another embodiment, the anti-CTLA-4 antibody is tremelimumab, also known as CP-675,206, which is an IgG2 monoclonal antibody which is described in U.S. Patent Application Publication No. 2012/263677, or PCT International Application Publication Nos. WO 2012/122444 or WO 2007/113648 A2.

In further embodiments of the treatment methods, compositions, kits, and uses of the present invention, the anti-CTLA4 antibody, or antigen binding fragment thereof, comprises: light chain CDRs comprising a sequence of amino acids as set forth in SEQ ID NOs: 24, 25 and 26 and heavy chain CDRs comprising a sequence of amino acids as set forth in SEQ ID NOs: 27, 28 and 29.

In other embodiments of the treatment methods, compositions, kits, and uses of the present invention, the anti-CTLA4 antibody is a monoclonal antibody, or antigen binding fragment thereof, which binds to human CTLA4 and comprises (a) a heavy chain variable region comprising an amino acid sequence as set forth in SEQ ID NO: 30 and (b) a light chain variable region comprising an amino acid sequence as set forth in SEQ ID NO: 31.

In one embodiment of the treatment methods, compositions, kits and uses of the invention, the anti-CTLA-4 antibody is a monoclonal antibody that comprises a heavy chain having the amino acid sequence set forth in SEQ ID NO:32 and a light chain comprising the amino acid sequence set forth in SEQ ID NO:33. In some embodiments, the anti-CTLA4 antibody is an antigen binding fragment of SEQ ID NO:32 and/or SEQ ID NO:33, wherein the antigen binding fragment specifically binds to CTLA4.

In one embodiment of the treatment methods, compositions, kits and uses of the invention, the anti-CTLA-4 antibody is any of the anti-CTLA-4 antibodies, or antigen binding fragments thereof, disclosed in International Application Publication No. WO 2016/015675 A1. In one embodiment, the anti-CTLA4 antibody is a monoclonal antibody which comprises the following CDR's:

CDRH1 comprising the amino acid sequence GFTFSDNW (SEQ ID NO:1);

CDRH2 comprising the amino acid sequence IRNKPYNYET (SEQ ID NO:2);

CDRH3 comprising the amino acid sequence TAQFAY (SEQ ID NO:3);

and/or

CDRL1 comprising the amino acid sequence ENIYGG (SEQ ID NO:4);

CDRL2 comprising the amino acid sequence GAT (SEQ ID NO:5); and

CDRL3 comprising an amino acid sequence selected from: QNVLRSPFT (SEQ ID NO:6); QNVLSRHPG (SEQ ID NO:7); or QNVLSSRPG (SEQ ID NO:8).

In one embodiment of the treatment methods, compositions, kits and uses of the invention, the anti-CTLA4 antibody is 8D2/8D2 (RE) or a variant thereof, 8D2H1L1 or a variant thereof, 8D2H2L2 or a variant thereof, 8D2H3L3 or a variant thereof, 8D2H2L15 or a variant thereof, or 8D2H2117 or a variant thereof.

In another embodiment of the treatment methods, compositions, kits and uses of the invention, the anti-CTLA4 antibody is a variant of 8D2/8D2 (RE), a variant of 8D2H1L1, a variant of 8D2H2L2, a variant of 8D2H2L15, or a variant of 8D2H2117, wherein the methionine (Met) at position 18 in the V_{H} chain amino acid sequence is independently substituted with an amino acid selected from: Leucine (Leu), Valine (Val), Isoleucine (Ile) or Alanine (Ala). In embodiments of the invention, the anti-CTLA4 antibody comprises the sequence of the 8D2H2L2 Variant 1 as set forth in the table above.

In another embodiment of the treatment methods, compositions, kits and uses of the invention, the anti-CTLA4 antibody is 8D2H2L2 Variant 1, having the full heavy chain amino acid sequence set forth in SEQ ID NO: 22 and the full light chain sequence set forth in SEQ ID NO: 23.

In one embodiment of the treatment methods, compositions, kits and uses of the invention, the anti-CTLA4 antibody is any of the anti-CTLA4 antibodies, or antigen binding fragments thereof, described as disclosed in International Application Publication No. WO 2018/035710 A1, published Mar. 1, 2018.

In certain embodiments of various methods, pharmaceutical compositions, kits, or uses provided herein, the anti-human CTLA4 antibody (e.g., monoclonal antibody) or antigen binding fragment thereof comprises a V_{L} CDR1 comprising an amino acid sequence comprising 80%, 85%, 90%, 95%, or 99% sequence identity to SEQ ID NO: 4, a V_{L} CDR2 comprising an amino acid sequence 80%, 85%, 90%, 95%, or 99% sequence identity to SEQ ID NO: 5, and a V_{L} CDR3 comprising an amino acid sequences 80%, 85%, 90%, 95%, or 99% sequence identity to SEQ ID NOS:6, 7, or 8, and a V_{H} CDR1, a V_{H} CDR2, and a V_{H} CDR3 comprising amino acid sequences comprising 80%, 85%, 90%, 95%, or 99% sequence identity to SEQ ID NOS: 1, 2, and 3, respectively.

Ordinarily, amino acid sequence variants of the anti-CTLA4 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

### 5. Methods of Treating Cancer Using a Combination of a PD-1 Antagonist, a CTLA4 Antagonist, and Lenvatinib or a Pharmaceutically Acceptable Salt Thereof

In another aspect, provided herein are methods of treating cancer (e.g., RCC) using a combination of a PD-1 antagonist, a CTLA4 antagonist, and lenvatinib or a pharmaceutically acceptable salt thereof described.

In some embodiments, the PD-1 antagonist is an anti-PD-1 antibody or antigen binding fragment thereof. In certain embodiments, the CTLA4 antagonist is an anti-CTLA4 antibody or antigen binding fragment thereof.

In certain embodiments, the method of treating cancer comprises administering to a human patient in need thereof:
(a) a PD-1 antagonist;
(b) a CTLA4 antagonist; and
(c) lenvatinib represented by Formula (I),

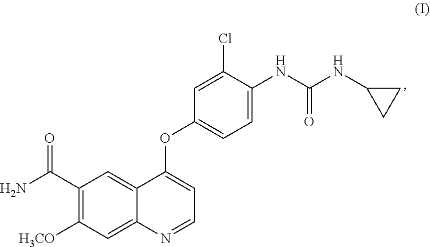 [SMILES: COc1cc2nccc(Oc3ccc(NC(=O)NC4CC4)c(Cl)c3)c2cc1C(N)=O]

or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is melanoma. In yet another embodiment, the cancer is melanoma and the patient has melanoma brain metastases. In yet another embodiment, the cancer is refractory melanoma.

In one embodiment, the cancer is advanced RCC. In another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In certain embodiments, provided herein is a method of treating RCC, comprising administering to a human patient in need thereof:
(a) a PD-1 antagonist;
(b) a CTLA4 antagonist; and
(c) lenvatinib represented by Formula (I),

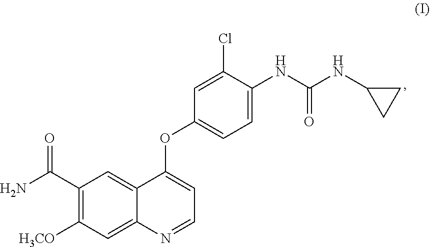 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is a method of treating advanced RCC, comprising administering to a human patient in need thereof:
(a) a PD-1 antagonist;
(b) a CTLA4 antagonist; and
(c) lenvatinib represented by Formula (I),

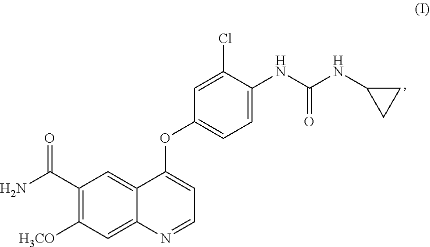 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is a method of treating metastatic RCC, comprising administering to a human patient in need thereof:
(a) a PD-1 antagonist;
(b) a CTLA4 antagonist; and
(c) lenvatinib represented by Formula (I),

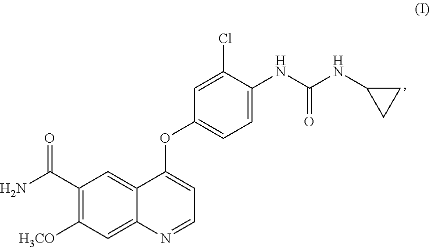 [SMILES: COc1cc2nccc(Oc3ccc(NC(=O)NC4CC4)c(Cl)c3)c2cc1C(N)=O]

or a pharmaceutically acceptable salt thereof.

In yet other embodiments, provided herein is a method of treating relapsed RCC, comprising administering to a human patient in need thereof:
(a) a PD-1 antagonist;
(b) a CTLA4 antagonist; and
(c) lenvatinib represented by Formula (I),

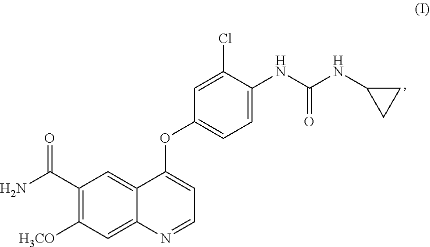 [SMILES: COc1cc2nccc(Oc3ccc(NC(=O)NC4CC4)c(Cl)c3)c2cc1C(N)=O]

or a pharmaceutically acceptable salt thereof.

In yet still other embodiments, provided herein is a method of treating refractory RCC, comprising administering to a human patient in need thereof:
(a) a PD-1 antagonist;
(b) a CTLA4 antagonist; and
(c) lenvatinib represented by Formula (I),

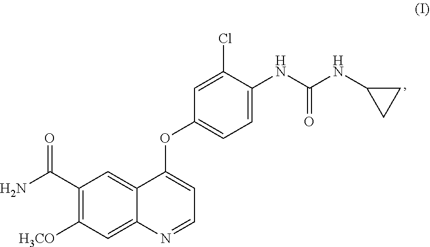 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is a method of treating relapsed and refractory RCC, comprising administering to a human patient in need thereof:
(a) a PD-1 antagonist;
(b) a CTLA4 antagonist; and
(c) lenvatinib represented by Formula (I),

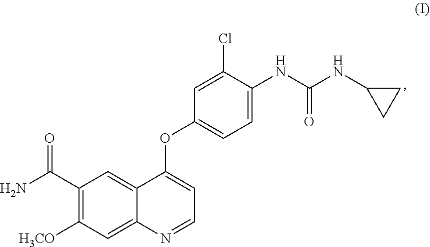 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is a method of treating melanoma, comprising administering to a human patient in need thereof:
(a) a PD-1 antagonist;
(b) a CTLA4 antagonist; and
(c) lenvatinib represented by Formula (I),

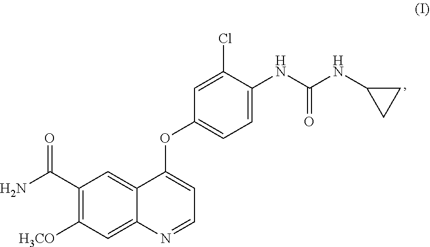 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is a method of treating refractory melanoma, comprising administering to a human patient in need thereof:
(a) a PD-1 antagonist;
(b) a CTLA4 antagonist; and
(c) lenvatinib represented by Formula (I),

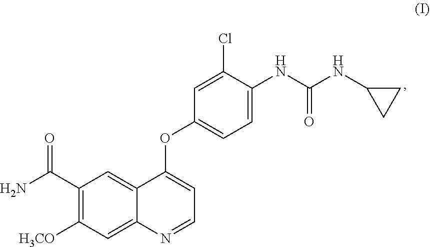 [SMILES: COc1cc2nccc(Oc3ccc(NC(=O)NC4CC4)c(Cl)c3)c2cc1C(N)=O]

or a pharmaceutically acceptable salt thereof.

In certain embodiments, the method of treating cancer comprises administering to a human patient in need thereof:
(a) a PD-1 antagonist as disclosed in Section V.2;
(b) a CTLA4 antagonist as disclosed in Section V.3; and
(c) lenvatinib or a pharmaceutically acceptable salt thereof.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the CTLA4 antagonist is an anti-human CTLA4 monoclonal antibody or antigen binding fragment thereof. In some embodiments, the anti-human CTLA4 monoclonal antibody is a human antibody. In other embodiments, the anti-human CTLA4 monoclonal antibody is a humanized antibody.

Thus, in certain embodiments, provided herein is a method for treating cancer, comprising administering to a human patient in need thereof:
(a) a human or humanized anti-human PD-1 monoclonal antibody or antigen binding fragment thereof,
(b) a human or humanized anti-human CTLA4 monoclonal antibody or antigen binding fragment thereof, and
(c) lenvatinib represented by Formula (I),

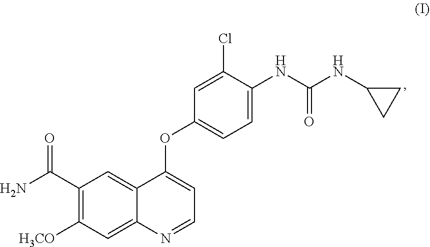 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is a method for treating cancer, comprising administering to a human patient in need thereof:
(a) a human anti-human PD-1 monoclonal antibody or antigen binding fragment thereof,
(b) a human anti-human CTLA4 monoclonal antibody or antigen binding fragment thereof, and
(c) lenvatinib represented by Formula (I),

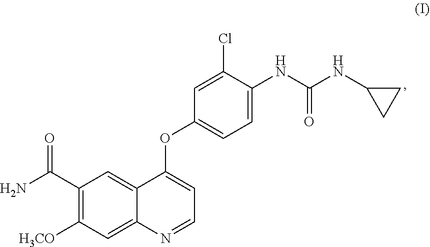 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is a method for treating cancer, comprising administering to a human patient in need thereof:
(a) a humanized anti-human PD-1 monoclonal antibody or antigen binding fragment thereof,
(b) a humanized anti-human CTLA4 monoclonal antibody or antigen binding fragment thereof, and
(c) lenvatinib represented by Formula (I),

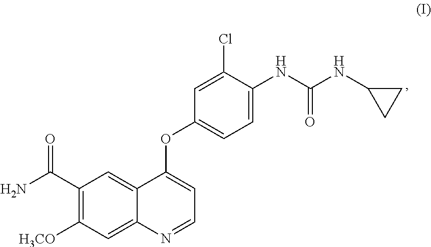 [SMILES: COc1cc2nccc(Oc3ccc(NC(=O)NC4CC4)c(Cl)c3)c2cc1C(N)=O]

or a pharmaceutically acceptable salt thereof.

In one embodiment of various methods provided herein, the anti-human PD-1 monoclonal antibody or antigen binding fragment thereof is pembrolizumab.

In another embodiment of various methods provided herein, the anti-human PD-1 monoclonal antibody or antigen binding fragment thereof is nivolumab.

In another embodiment of various methods provided herein, the anti-human PD-1 monoclonal antibody or antigen binding fragment thereof is cemiplimab.

In certain embodiments of various methods provided herein, the anti-human CTLA4 monoclonal antibody or antigen binding fragment thereof comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

In some embodiments of various methods provided herein, the anti-human CTLA4 monoclonal antibody or antigen binding fragment thereof comprises a V_{L} region comprising an amino acid sequence as set forth in SEQ ID NO: 14, and a V_{H} region comprising an amino acid sequence as set forth in SEQ ID NO:15.

In other embodiments of various methods provided herein, the anti-human CTLA4 monoclonal antibody or antigen binding fragment thereof comprises a light chain comprising or consisting of an amino acid sequence as set forth in SEQ ID NO:23 and a heavy chain comprising or consisting of an amino acid sequence as set forth in SEQ ID NO:22.

Thus, in one specific embodiment of various methods provided herein, the method for treating cancer comprises administering to a human patient in need thereof:
(a) pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating cancer comprises administering to a human patient in need thereof:
(a) nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating cancer comprises administering to a human patient in need thereof:
(a) cemiplimab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating RCC comprises administering to a human patient in need thereof:
(a) pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating RCC comprises administering to a human patient in need thereof:
(a) nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID Nos: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating RCC comprises administering to a human patient in need thereof:
(a) cemiplimab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one embodiment, the RCC is advanced RCC. In another embodiment, the RCC is metastatic RCC. In yet another embodiment, the RCC is relapsed RCC. In still another embodiment, the RCC is refractory RCC. In yet still another embodiment, the RCC is relapsed and refractory RCC.

In one specific embodiment of various methods provided herein, the method for treating melanoma comprises administering to a human patient in need thereof:
(a) pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating melanoma comprises administering to a human patient in need thereof:
(a) nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID Nos: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment of various methods provided herein, the method for treating melanoma comprises administering to a human patient in need thereof:
(a) cemiplimab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one embodiment, the melanoma is refractory melanoma.

### 6. Dosing and Administration

Further provided herein are dosing regimens and routes of administration for treating cancer (e.g., RCC) using a combination of a PD-1 antagonist (e.g., an anti-PD-1 monoclonal antibody or antigen binding fragment thereof), a CTLA4 antagonist (e.g., an anti-CTLA4 monoclonal antibody or antigen binding fragment thereof), and a multi-RTK inhibitor (e.g., lenvatinib or a pharmaceutically acceptable salt thereof).

The anti-PD-1 monoclonal antibody or antigen binding fragment thereof, the anti-CTLA4 monoclonal antibody or antigen binding fragment thereof, or lenvatinib or a pharmaceutically acceptable salt thereof disclosed herein may be administered by doses administered, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. In certain embodiments, the doses are administered orally. A total dose for a treatment interval is generally at least 0.05 g/kg body weight, more generally at least 0.2 g/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more. Doses may also be provided to achieve a pre-determined target concentration of the antibody (e.g., anti-PD-1 antibody) or antigen binding fragment thereof in the subject's serum, such as 0.1, 0.3, 1, 3, 10, 30, 100, 300 μg/mL or more.

In some embodiments, the anti-PD-1 monoclonal antibody or antigen binding fragment thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at 10, 20, 50, 80, 100, 200, 300, 400, 500, 1000 or 2500 mg/subject. In some specific methods, the dose of the anti-PD-1 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In one embodiment, the anti-CTLA4 antibody or antigen binding fragment thereof comprises: (a) light chain CDRs comprising a sequence of amino acids as set forth in SEQ ID NOs: 4, 5 and 6 and heavy chain CDRs comprising a sequence of amino acids as set forth in SEQ ID NOs: 1, 2, and 3; (b) light chain CDRs comprising a sequence of amino acids as set forth in SEQ ID NOs: 4, 5, and 7 and heavy chain CDRs comprising a sequence of amino acids as set forth in SEQ ID NOs: 1, 2, and 3 or (c) light chain CDRs comprising a sequence of amino acids as set forth in SEQ ID NOs: 4, 5 and 8 and heavy chain CDRs comprising a sequence of amino acids as set forth in SEQ ID NOs: 1, 2, and 3.

In one embodiment, the anti-CTLA4 antibody, or antigen binding fragment thereof, comprises light chain CDRs comprising a sequence of amino acids as set forth in SEQ ID NOs: 4, 5, and 6 and heavy chain CDRs comprising a sequence of amino acids as set forth in SEQ ID NOs: 1, 2, and 3.

In some embodiments, the anti-CTLA4 monoclonal antibody or antigen binding fragment thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at 1, 10, 20, 25, 30, 40, 50, 60, 75, 80, 90, 100, 110, 120, 125, 130, 140, 150, 160, 170, 175, 180, 190, 200, 500, 1000, or 2500 mg/subject. In another embodiment, the anti-CTLA4 monoclonal antibody or antigen binding fragment thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at 1, 10, 20, 25, 30, 40, 50, 60, 75, 80, 90, 100, 110, 120, 125, 130, 140, 150, 160, 170, 175, 180, 190, 200 mg/subject.

In some specific methods, the dose of the anti-CTLA4 monoclonal antibody or antigen binding fragment thereof is from about 1 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-CTLA4 monoclonal antibody or antigen binding fragment thereof is 1 mg, 5 mg, 10 mg, 15 mg, 20 mg, 30 mg, 40 mg, 45 mg, 50 mg, 75 mg, 100 mg, 125 mg, 150 mg, 175 mg, 200 mg, 225 mg, 240 mg, 250 mg, 300 mg, 400 mg, or 500 mg.

In another embodiment, the dose of the anti-CTLA4 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-CTLA4 monoclonal antibody or antigen binding fragment thereof is from about 1.0 mg/ml, 1.25 mg/ml, 1.4 mg/ml, 2.0 mg/ml, 2.5 mg/ml, 2.9 mg/ml, 5 mg/ml, 7.9 mg/ml, 10 mg/ml, 12.5 mg/ml, 25 mg/ml, 50 mg/ml, 75 mg/ml, 100 mg/ml, 125 mg/ml, 150 mg/ml, 175 mg/ml, or 200 mg/ml.

In some specific methods, the dose of the anti-CTLA4 monoclonal antibody or antigen binding fragment thereof is from about 1 mg to about 200 mg, from about 1 mg to about 100 mg. In some embodiments, the dose of the anti-CTLA4 monoclonal antibody or antigen binding fragment thereof is 1 mg, 2.9 mg, 5 mg, 10 mg, 25 mg, or 50 mg. In one embodiment, the dose of the anti-CTLA4 monoclonal antibody or antigen binding fragment thereof is 25 mg.

In some embodiments of various methods described herein, the anti-human PD-1 monoclonal antibody or antigen binding fragment thereof is pembrolizumab, the human patient is administered 200 mg, 240 mg, 400 mg, 480 mg, 720 mg, or 2 mg/kg pembrolizumab, and pembrolizumab is administered once every three or six weeks. In one embodiment, the human patient is administered 200 mg pembrolizumab once every three weeks. In one embodiment, the human patient is administered 240 mg pembrolizumab once every three weeks. In one embodiment, the human patient is administered 2 mg/kg pembrolizumab once every three weeks. In one embodiment, the human patient is administered 400 mg pembrolizumab once every three weeks.

In certain embodiments of various methods described herein, the anti-human PD-1 monoclonal antibody or antigen binding fragment thereof is pembrolizumab, the human patient is administered 400 mg pembrolizumab, and pembrolizumab is administered once every six weeks.

In some embodiments of various methods described herein, the anti-human PD-1 monoclonal antibody or antigen binding fragment thereof is pembrolizumab, the human patient is administered 200 mg, 240 mg, 400 mg, 480 mg, 720 mg, or 2 mg/kg pembrolizumab, and pembrolizumab is administered once every six weeks. In one embodiment, the human patient is administered 200 mg pembrolizumab once every six weeks. In one embodiment, the human patient is administered 240 mg pembrolizumab once every six weeks. In one embodiment, the human patient is administered 400 mg pembrolizumab once every six weeks. In one embodiment, the human patient is administered 480 mg pembrolizumab once every six weeks. In one embodiment, the human patient is administered 720 mg pembrolizumab once every six weeks. In one embodiment, the human patient is administered 2 mg/kg pembrolizumab once every six weeks.

In some embodiments of the invention, the anti-PD-1 antibody, or antigen binding fragment thereof, and the anti-CTLA4 antibody, or antigen binding fragment thereof, are administered to the patient once every approximately six weeks for 12 weeks or more. In other embodiments, the anti-PD-1 antibody, or antigen binding fragment and the anti-CTLA4 antibody, or antigen binding fragment thereof, are administered to the patient once every six weeks for 18 weeks or more, 24 weeks or more, 30 weeks or more, 36 weeks or more, 42 weeks or more, 48 weeks or more, 54 weeks or more, 60 weeks or more, 66 weeks or more, 72 weeks or more, 78 weeks or more, 84 weeks or more, or 90 weeks or more. In one embodiment, the administration occurs on the same day.

In a sub-embodiment, the anti-PD-1 antibody, or antigen binding fragment thereof, and the anti-CTLA4 antibody, or antigen binding fragment thereof, are administered on the same day simultaneously (e.g., in a single formulation or concurrently as separate formulations). In an alternative embodiment, the anti-PD-1 antibody or antigen binding fragment thereof and the anti-CTLA4 antibody or antigen binding fragment thereof are administered sequentially on the same day (e.g., as separate formulations), in either order. In one embodiment of same day sequential administration, the anti-PD-1 antibody or antigen binding fragment thereof is administered first. In another embodiment of same day sequential administration, the anti-CTLA4 antibody or antigen binding fragment thereof is administered first. In certain embodiments of various methods described herein, the anti-human PD-1 monoclonal antibody or antigen binding fragment thereof is pembrolizumab, the human patient is administered 200 mg pembrolizumab, and pembrolizumab is administered once every three weeks. In certain embodiments of various methods described herein, the anti-human PD-1 monoclonal antibody or antigen binding fragment thereof is pembrolizumab, the human patient is administered 400 mg pembrolizumab, and pembrolizumab is administered once every six weeks.

In other embodiments of various methods described herein, the anti-human PD-1 monoclonal antibody or antigen binding fragment thereof is nivolumab, the human patient is administered 240 mg or 3 mg/kg nivolumab, and nivolumab is administered once every two weeks. In one specific embodiment, the human patient is administered 240 mg nivolumab once every two weeks. In one specific embodiment, the human patient is administered 3 mg/kg nivolumab once every two weeks. In other embodiments of various methods described herein, the anti-human PD-1 monoclonal antibody or antigen binding fragment thereof is nivolumab, the human patient is administered 480 mg nivolumab, and nivolumab is administered once every four weeks.

In yet other embodiments of various methods described herein, the anti-human PD-1 monoclonal antibody or antigen binding fragment thereof is cemiplimab, the human patient is administered 350 mg cemiplimab, and cemiplimab is administered once every three weeks.

In still other embodiments of various methods described herein, the anti-human CTLA4 monoclonal antibody or antigen binding fragment thereof comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively, the human patient is administered from about 1 to about 200 mg anti-human CTLA4 antibody, and anti-human CTLA4 antibody is administered once every six weeks. In still other embodiments of various methods described herein, the anti-human CTLA4 monoclonal antibody or antigen binding fragment thereof comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively, the human patient is administered 1-100 mg anti-human CTLA4 antibody, and anti-human CTLA4 antibody is administered once every six weeks. In one specific embodiment, the human patient is administered 1.4 mg/ml anti-human CTLA4 antibody once every six weeks. In one specific embodiment, the human patient is administered 25 mg anti-human CTLA4 antibody once every six weeks. In one specific embodiment, the human patient is administered 50 mg anti-human CTLA4 antibody once every six weeks.

In certain embodiments, lenvatinib or a pharmaceutically acceptable salt thereof is administered orally. In some embodiments, lenvatinib or a pharmaceutically acceptable salt thereof is administered at a daily dose of 8, 10, 12, 14, 18, 20, or 24 mg, of lenvatinib.

Thus, in some embodiments of various methods provided herein, the human patient is administered:
(a) 200 mg, 240 mg, 400 mg, or 2 mg/kg pembrolizumab;
(b) 1-200 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 8, 10, 12, 14, 18, 20, or 24 mg lenvatinib;
wherein each (a) and (b) are administered once every three or six weeks, and wherein (c) is administered daily.

In certain embodiments of various methods provided herein, the human patient is administered:
(a) 200 mg, 240 mg, 400 mg, 2 mg/kg or 22. mg/mL pembrolizumab;
(b) 1-200 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 8, 10, 12, 14, 18, 20, or 24 mg lenvatinib;
wherein each of (a) and (b) is administered once every six weeks; and wherein (c) is administered daily.

In certain embodiments of various methods provided herein, the human patient is administered:
(a) 200 mg pembrolizumab;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 20 mg lenvatinib;
wherein each of (a) and (b) is administered once every six weeks; and
wherein (c) is administered daily.

In certain embodiments of various methods provided herein, the human patient is administered:
(a) 240 mg pembrolizumab;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 20 mg lenvatinib;
wherein each of (a) and (b) is administered once every six weeks; and
wherein (c) is administered daily.

In certain embodiments of various methods provided herein, the human patient is administered:
(a) 2 mg/kg pembrolizumab;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a comprising amino acid sequences as set forth in SEQ ID NOs:1, 2, and 3, respectively; and
(c) 20 mg lenvatinib;
wherein each of (a) and (b) is administered once every six weeks; and
wherein (c) is administered daily.

In certain embodiments of various methods provided herein, the human patient is administered:
(a) 400 mg pembrolizumab;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 20 mg lenvatinib;
wherein (a) is administered once every six weeks; wherein (b) is administered once every six weeks; and wherein (c) is administered daily.

In certain embodiments of various methods provided herein, the human patient is administered:
(a) 200 mg pembrolizumab;
(b) 1.4 mg/ml an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 20 mg lenvatinib;
wherein each of (a) and (b) is administered once every six weeks; and
wherein (c) is administered daily.

In certain embodiments of various methods provided herein, the human patient is administered:
(a) 400 mg pembrolizumab;
(b) 50 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in NOs: 1, 2, and 3, respectively; and
(c) 20 mg lenvatinib;
wherein each of (a) and (b) is administered once every three weeks; and
wherein (c) is administered daily.

In certain embodiments of various methods provided herein, the human patient is administered:
(a) 400 mg pembrolizumab;
(b) 100 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in NOs: 1, 2, and 3, respectively; and
(c) 20 mg lenvatinib;
wherein each of (a) and (b) is administered once every six weeks; and
wherein (c) is administered daily.

In certain embodiments of various methods provided herein, the human patient is administered:
(a) 400 mg pembrolizumab;
(b) 200 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in NOs: 1, 2, and 3, respectively; and
(c) 20 mg lenvatinib;
wherein each of (a) and (b) is administered once every six weeks; and
wherein (c) is administered daily.

In certain embodiments of various methods provided herein, the human patient is administered:
(a) 400 mg pembrolizumab;
(b) 225 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in NOs: 1, 2, and 3, respectively; and
(c) 14 mg lenvatinib;
wherein each of (a) and (b) is administered once every six weeks; and
wherein (c) is administered daily.

In certain embodiments of various methods provided herein, the human patient is administered:
(a) 400 mg pembrolizumab;
(b) 25 mg of an anti-human CTLA4 antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in NOs: 1, 2, and 3, respectively; and
(c) 10 mg lenvatinib;
wherein each of (a) and (b) is administered once every six weeks; and
wherein (c) is administered daily.

In some embodiments, at least one of the therapeutic agents (e.g., the anti-PD-1 monoclonal antibody or binding fragment thereof, the anti-CTLA4 monoclonal antibody or binding fragment thereof, or lenvatinib) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-PD-1 monoclonal antibody or binding fragment thereof, the anti-CTLA4 monoclonal antibody or binding fragment thereof, or lenvatinib) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a biotherapeutic or chemotherapeutic agent, i.e., is treatment-naïve. In other embodiments, the combination therapy is administered to a patient who failed to achieve a sustained response after prior therapy with the biotherapeutic or chemotherapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with one or more of the therapeutic agents in the combinations disclosed herein.

The one or more additional active agents may be co-administered with the anti-PD-1 monoclonal antibody or antigen binding fragment thereof, the anti-CTLA4 monoclonal antibody or antigen binding fragment thereof, or lenvatinib or a pharmaceutically acceptable salt thereof. The additional active agent(s) can be administered in a single dosage form with one or more co-administered agent selected from the anti-PD-1 monoclonal antibody or antigen binding fragment thereof, the anti-CTLA4 monoclonal antibody or antigen binding fragment thereof, and lenvatinib or a pharmaceutically acceptable salt thereof. The additional active agent(s) can also be administered in separate dosage form(s) from the dosage forms containing the anti-PD-1 monoclonal antibody or antigen binding fragment thereof, the anti-CTLA4 monoclonal antibody or antigen binding fragment thereof, or lenvatinib or a pharmaceutically acceptable salt thereof.

### 7. Pharmaceutical Compositions

In yet another aspect, provided herein are pharmaceutical compositions comprising the therapeutic agents disclosed herein (e.g., a PD-1 antagonist, a CTLA4 antagonist, and lenvatinib).

In certain embodiments, the pharmaceutical composition further comprises a pharmaceutically acceptable carrier.

The pharmaceutical compositions comprising an anti-human PD-1 monoclonal antibody or antigen binding fragment thereof, an anti-human CTLA4 monoclonal antibody or antigen binding fragment thereof, and lenvatinib or pharmaceutically acceptable salt thereof, can be prepared for storage by mixing the antibodies having the desired degree of purity with optionally physiologically acceptable carriers, excipients, or stabilizers (see, e.g., Remington, Remington's Pharmaceutical Sciences (18^{th} ed. 1980)) in the form of aqueous solutions or lyophilized or other dried forms.

The pharmaceutically acceptable carriers, excipients, or stabilizers are non-toxic to the cell or mammalian being exposed thereto at the dosage and concentrations employed. Often the pharmaceutically acceptable carrier is an aqueous pH buffered solution. Examples of pharmaceutically acceptable carriers include buffers, such as phosphate, citrate, acetate, and other organic acids; antioxidants, such as ascorbic acid; low molecular weight (e.g., fewer than about 10 amino acid residues) polypeptide; proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The pharmaceutically acceptable carriers can also refer to a diluent, adjuvate (e.g., Freund's adjuvate (complete or incomplete)), excipient, or vehicle. Such carriers can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsions, tablets, pills, capsules, powders, sustained-release formulations, and the like.

### 8. Kits

In still another aspect, provided herein are kits comprising the therapeutic agents disclosed herein (e.g., a PD-1 antagonist, a CTLA4 antagonist, and lenvatinib) or pharmaceutical compositions thereof, packaged into suitable packaging material. A kit optionally includes a label or packaging insert that include a description of the components or instructions for use in vitro, in vivo, or ex vivo, of the components therein.

In some embodiments, the kit comprises
(a) a PD-1 antagonist;
(b) a CTLA4 antagonist; and
(c) lenvatinib or a pharmaceutically acceptable salt thereof.

In certain embodiments, the kit further comprises instructions for administering to a human patient the PD-1 antagonist, the CTLA4 antagonist, and lenvatinib or a pharmaceutically acceptable salt thereof.

In some embodiments, the PD-1 antagonist is an anti-PD-1 monoclonal antibody or antigen-binding fragment thereof. In some embodiments, the PD-1 antagonist is an anti-PD-L1 monoclonal antibody or antigen-binding fragment thereof. In some embodiments, the CTLA4 antagonist is an anti-CTLA4 monoclonal antibody or antigen-binding fragment thereof.

In one embodiment, the kit comprises: (a) one or more dosages of an anti-PD-1 monoclonal antibody or antigen binding fragment thereof; (b) one or more dosages of an anti-CTLA4 monoclonal antibody or antigen binding fragment thereof; (c) one or more dosages of lenvatinib or a pharmaceutically acceptable salt thereof; and (d) instructions for administering to a human patient the anti-human PD-1 monoclonal antibody or antigen binding fragment thereof, the anti-human CTLA4 monoclonal antibody or antigen binding fragment thereof, and lenvatinib or a pharmaceutically acceptable salt thereof.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

The dosages for the anti-PD-1 monoclonal antibody, the anti-CTLA4 monoclonal antibody, or lenvatinib or a pharmaceutically acceptable salt thereof described in section V.6 can be used in various kits herein. In some embodiments, a kit comprises dosages of each component sufficient for a certain period of treatment (e.g., 3, 6, 12, or 24 weeks, etc.). For example, a kit can comprise a dosage of 200 mg pembrolizumab, 1 dosage of 25 mg anti-CTLA4 antibody, and 21 dosages of 20 mg lenvatinib (or equivalent amount of a pharmaceutically acceptable salt of lenvatinib), which are sufficient for a 3-week treatment. Or, a kit can also comprise a dosage of 400 mg pembrolizumab, 1 dosage of 25 mg anti-CTLA4 antibody, and 42 dosages of 20 mg lenvatinib (or equivalent amount of a pharmaceutically acceptable salt of lenvatinib), which are sufficient for a 6-week treatment.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### 9. Uses of a Therapeutic Combination for Treating Cancer

In still another aspect, provided herein are uses of a therapeutic combination for treating cancer (e.g., melanoma or RCC) in a human patient, wherein the therapeutic combination comprises:
(a) a PD-1 antagonist;
(b) a CTLA4 antagonist; and
(c) lenvatinib represented by Formula (I),

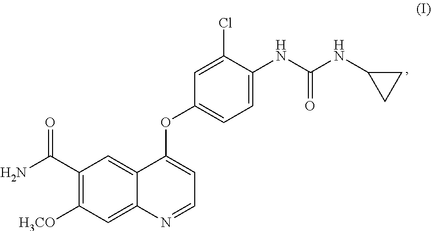 [SMILES: COc1cc2nccc(Oc3ccc(NC(=O)NC4CC4)c(Cl)c3)c2cc1C(N)=O]

or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is melanoma.

In one embodiment, the cancer is advanced RCC. In another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In one embodiment, provided herein is use of a therapeutic combination for treating RCC in a human patient, wherein the therapeutic combination comprises:
(a) a PD-1 antagonist;
(b) a CTLA4 antagonist; and
(c) lenvatinib represented by Formula (I),

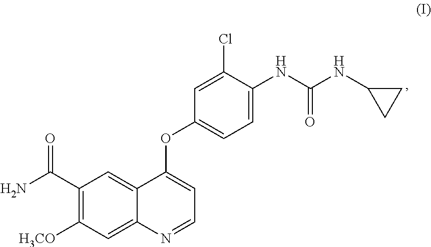 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is use of a therapeutic combination for treating advanced RCC in a human patient, wherein the therapeutic combination comprises:
(a) a PD-1 antagonist;
(b) a CTLA4 antagonist; and
(c) lenvatinib represented by Formula (I),

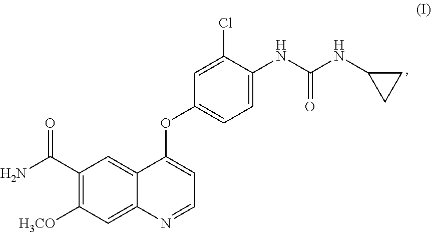 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating metastatic RCC in a human patient, wherein the therapeutic combination comprises:
(a) a PD-1 antagonist;
(b) a CTLA4 antagonist; and
(c) lenvatinib represented by Formula (I),

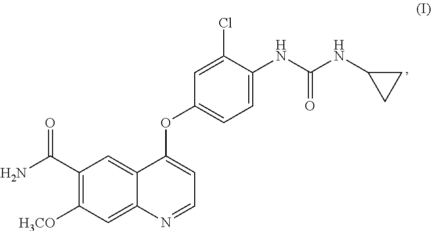 [SMILES: COc1cc2nccc(Oc3ccc(NC(=O)NC4CC4)c(Cl)c3)c2cc1C(N)=O]

or a pharmaceutically acceptable salt thereof.

In yet other embodiments, provided herein is use of a therapeutic combination for treating relapsed RCC in a human patient, wherein the therapeutic combination comprises:
(a) a PD-1 antagonist;
(b) a CTLA4 antagonist; and
(c) lenvatinib represented by Formula (I),

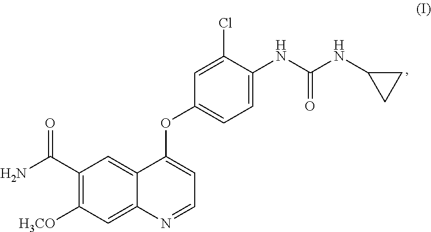 [SMILES: COc1cc2nccc(Oc3ccc(NC(=O)NC4CC4)c(Cl)c3)c2cc1C(N)=O]

or a pharmaceutically acceptable salt thereof.

In yet still other embodiments, provided herein is use of a therapeutic combination for treating refractory RCC in a human patient, wherein the therapeutic combination comprises:
(a) a PD-1 antagonist;
(b) a CTLA4 antagonist; and
(c) lenvatinib represented by Formula (I),

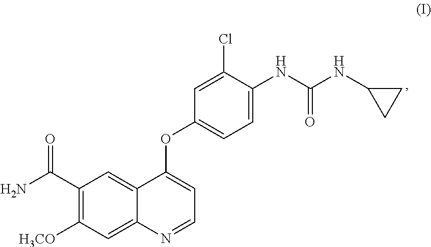 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating relapsed and refractory RCC in a human patient, wherein the therapeutic combination comprises:
(a) a PD-1 antagonist;
(b) a CTLA4 antagonist; and
(c) lenvatinib represented by Formula (I),

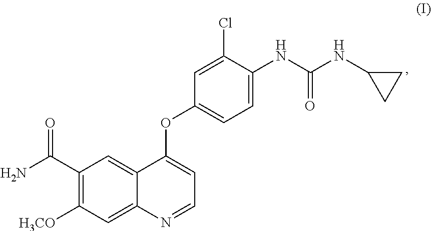 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating melanoma in a human patient, wherein the therapeutic combination comprises:
(a) a PD-1 antagonist;
(b) a CTLA4 antagonist; and
(c) lenvatinib represented by Formula (I),

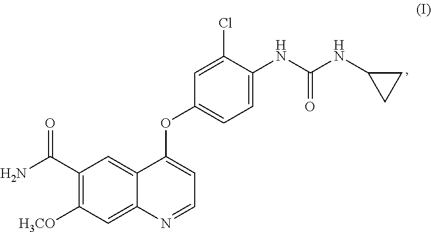 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating refractory melanoma in a human patient, wherein the therapeutic combination comprises:
(a) a PD-1 antagonist;
(b) a CTLA4 antagonist; and
(c) lenvatinib represented by Formula (I),

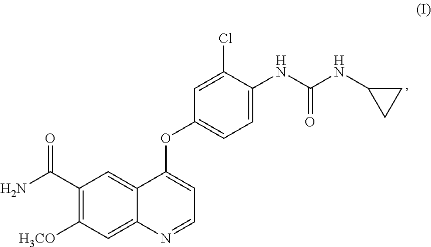 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating melanoma brain metastases melanoma in a human patient, wherein the therapeutic combination comprises:
(a) a PD-1 antagonist;
(b) a CTLA4 antagonist; and
(c) lenvatinib represented by Formula (I),

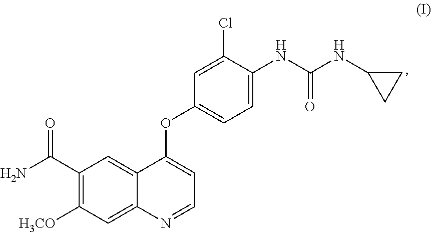 [SMILES: COc1cc2nccc(Oc3ccc(NC(=O)NC4CC4)c(Cl)c3)c2cc1C(N)=O]

or a pharmaceutically acceptable salt thereof.

In still other embodiments, provided herein is use of a therapeutic combination for treating cancer, wherein the therapeutic combination comprises:
(a) a PD-1 antagonist as disclosed in Section V.2;
(b) a CTLA4 antagonist as disclosed in Section V.3; and
(c) lenvatinib represented by Formula (I),

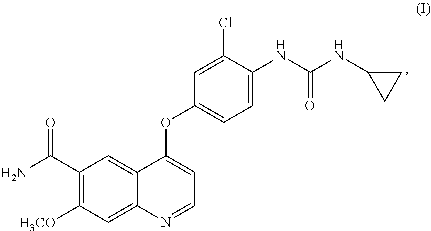 [SMILES: COc1cc2nccc(Oc3ccc(NC(=O)NC4CC4)c(Cl)c3)c2cc1C(N)=O]

or a pharmaceutically acceptable salt thereof.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the CTLA4 antagonist is an anti-human CTLA4 monoclonal antibody or antigen binding fragment thereof. In some embodiments, the anti-human CTLA4 monoclonal antibody is a human antibody. In other embodiments, the anti-human CTLA4 monoclonal antibody is a humanized antibody.

Thus, in certain embodiments, provided herein is use of a therapeutic combination for treating cancer, wherein the therapeutic combination comprises:
(a) a human or humanized anti-human PD-1 monoclonal antibody or antigen binding fragment thereof,
(b) a human or humanized anti-human CTLA4 monoclonal antibody or antigen binding fragment thereof, and
(c) lenvatinib represented by Formula (I),

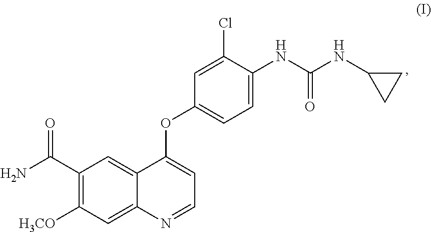 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is use of a therapeutic combination for treating cancer, wherein the therapeutic combination comprises:
(a) a human anti-human PD-1 monoclonal antibody or antigen binding fragment thereof;
(b) a human anti-human CTLA4 monoclonal antibody or antigen binding fragment thereof, and
(c) lenvatinib represented by Formula (I),

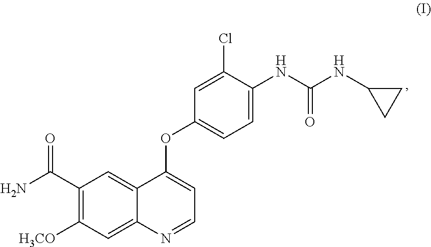 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating cancer, wherein the therapeutic combination comprises:
(a) a humanized anti-human PD-1 monoclonal antibody or antigen binding fragment thereof,
(b) a humanized anti-human CTLA4 monoclonal antibody or antigen binding fragment thereof, and
(c) lenvatinib represented by Formula (I),

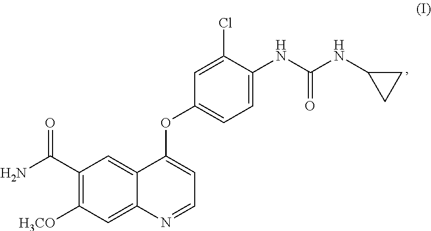 [SMILES: COc1cc2nccc(Oc3ccc(NC(=O)NC4CC4)c(Cl)c3)c2cc1C(N)=O]

or a pharmaceutically acceptable salt thereof.

In some embodiments of various uses provided herein, the anti-PD-1 monoclonal antibody or antigen binding fragment thereof is pembrolizumab. In some embodiments of various uses provided herein, the anti-PD-1 monoclonal antibody or antigen binding fragment thereof is nivolumab. In some embodiments of various uses provided herein, the anti-PD-1 monoclonal antibody or antigen binding fragment thereof is cemiplimab.

In certain embodiments of various uses provided herein, the anti-human CTLA4 monoclonal antibody or antigen binding fragment thereof comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

In some embodiments of various uses provided herein, the anti-human CTLA4 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:15.

In other embodiments of various uses provided herein, the anti-human CTLA4 monoclonal antibody or antigen binding fragment thereof comprises a light chain comprising or consisting of an amino acid sequence as set forth in SEQ ID NO:23 and a heavy chain comprising or consisting of an amino acid sequence as set forth in SEQ ID NO:22.

Thus, in one specific embodiment, provided herein is use of a therapeutic combination for treating cancer, wherein the therapeutic combination comprises:
(a) pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment, provided herein is use of a therapeutic combination for treating cancer, wherein the therapeutic combination comprises:
(a) nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment, provided herein is use of a therapeutic combination for treating cancer, wherein the therapeutic combination comprises:
(a) cemiplimab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment, provided herein is use of a therapeutic combination for treating RCC, wherein the therapeutic combination comprises:
(a) pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment, provided herein is use of a therapeutic combination for treating RCC, wherein the therapeutic combination comprises:
(a) nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one specific embodiment, provided herein is use of a therapeutic combination for treating RCC, wherein the therapeutic combination comprises:
(a) cemiplimab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) lenvatinib, or a pharmaceutically acceptable salt thereof.

In one embodiment, the RCC is advanced RCC. In another embodiment, the RCC is metastatic RCC. In yet another embodiment, the RCC is relapsed RCC. In still another embodiment, the RCC is refractory RCC. In yet still another embodiment, the RCC is relapsed and refractory RCC.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments.

### EXAMPLES

The examples in this section are offered by way of illustration, and not by way of limitation.

### Example 1: Clinical Trial of Administering an Anti-PD-1 Antibody in Combination with an Anti-CTLA4 Antibody, and Lenvatinib in Melanoma Patients

The design overview for the Phase 1/2 open-label rolling arm umbrella platform design of a CTLA4 antagonist (MK-1308) in combination with pembrolizumab and lenvatinib in patients with Melanoma. Participants with Melanoma undergo ICF screening (no molecular testing required), and proceed to substudy 02A where participants have PD-1 refractory melanoma.

This study is a phase 1/2, rolling arm, multicenter, open-label, adaptive design study that will evaluate within this study the efficacy of investigational agents for the treatment of PD-1 refractory melanoma.

Participants will be administered 400 mg Pembrolizumab once every six weeks (Q6W) by IV in combination with a CTLA4 antagonist (MK-1308) (at a fixed dose of 25 mg Q6W), and lenvatinib (20 mg QD (once daily) PO (by mouth, orally)). Lenvatinib is administered at the dose specified orally every day. When more than 1 agent is to be given on the same day, the order of administration is pembrolizumab and then lenvatinib.

At the time of the Oct. 28, 2020 data cutoff, a total of 14 participants were enrolled in the triplet combination of pembrolizumab (400 mg) Q6W, MK-1308 (25 mg) Q6W, and Lenvatinib (20 mg) QD. 10 of these participants were enrolled in the Safety Lead-in phase and 4 of these participants were enrolled in the Efficacy phase. 7 of the participants in the safety lead-in phase completed DLT monitoring window. All 14 participants were analyzed. There were no dose limiting toxicities (DLT's) in the Safety Lead-in phase, and we were able to establish recommended phase 2 dosing at the 20 mg Lenvatinib dose. This triplet combination with lenvatinib at 20 mg has been generally well tolerated. 4 patients (28.6%) experienced grade 3-5 drug related AE's, all grade 3, and there were 2 SAE's. 2 of the 4 patients (14.3%) that experienced grade 3-5 drug-related AE's experienced hypertension, 1 patient (7.1%) experienced increased blood alkaline phosphatase, one patient (7.1%) experienced an embolism, and 1 patient (7.1%) experienced increased gamma-glutamyltransferase.

Out of the 14 study participants, 4 patients experienced Grade 3 drug-related adverse events, no Grade 4 and 5 drug-related adverse events have been observed. 2 participants had adverse events of special interest (AEOSI). One participant had hypothyroidism and one participant had pancreatitis. Out of the total 14 treated participants, preliminary data shows that 8 (57.1%) had one or more drug-related AE's.

Two patients have reduced their doses of Lenvatinib, one discontinued Lenvatinib completely due to toxicities—Participant continued treatment with MK1308+ pembrolizumab. One death was reported in this triplet combination efficacy phase after the data cutoff.

There are 3 predetermined dose levels of lenvatinib that may be explored (Dose Level 0, Dose Level −1, Dose Level −2).

Patient characteristics are found in Table 4. Additional patient data from the Safety Lead-in phase can be found in the following tables.

TABLE 4
Participant Characteristics (APaT Population) by Treatment Arm

| | MK-3475 400 mg Q6W + | |
|---|---|---|
| | MK-1308 25 mg Q6W + | |
| | Lenvatinib 20 mg QD | |
| | n | (%) |
| Participants in population | 14 | |

Sex

| | | |
|---|---|---|
| Male | 12 | 85.7 |
| Female | 2 | 14.3 |

Age (Years)

| | | |
|---|---|---|
| <65 | 8 | 57.1 |
| ≥65 | 6 | 42.9 |
| Range | 33 to 78 | |

Race

| | | |
|---|---|---|
| White | 12 | 85.7 |
| Missing | 2 | 14.3 |

Ethnicity

| | | |
|---|---|---|
| Hispanic or Latino | 1 | 7.1 |
| Not Hispanic Or Latino | 12 | 85.7 |
| Not Reported | 1 | 7.1 |

ECOG

| | | |
|---|---|---|
| 0 | 8 | 57.1 |
| 1 | 6 | 42.9 |

LDH

| | | |
|---|---|---|
| ≤ULN | 12 | 85.7 |
| >ULN but <2X ULN | 1 | 7.1 |
| ≥2X ULN | 1 | 7.1 |

Overall Cancer Stage at Enrollment

| | | |
|---|---|---|
| IIIC | 2 | 14.3 |
| IV | 12 | 85.7 |

Brain Metastasis*

| | | |
|---|---|---|
| No | 14 | 100.0 |

Metastatic Staging

| | | |
|---|---|---|
| MO | 2 | 14.3 |
| M1a(0) | 4 | 28.6 |
| M1b(0) | 1 | 7.1 |
| M1c(0) | 5 | 35.7 |
| M1c(1) | 2 | 14.3 |

Baseline Tumor Size (mm) based on Central Evaluation

| | |
|---|---|
| Participants with Data | 7 |
| Median | 20 |
| Range | 10.0 to 31.0 |

Prior Lines of Therapy

| | | |
|---|---|---|
| One Line | 11 | 78.6 |
| Two Lines | 3 | 21.4 |

Prior Adjuvant Systemic Immunotherapy

| | | |
|---|---|---|
| Yes | 12 | 85.7 |
| No | 2 | 14.3 |

Prior Systemic Immunotherapy for Progressive/Relapsed Disease

| | | |
|---|---|---|
| Yes | 2 | 14.3 |
| No | 12 | 85.7 |

Prior Adjuvant Systemic BRAF/MEK Inhibitor

| | | |
|---|---|---|
| Yes | 1 | 7.1 |
| No | 13 | 92.9 |

Prior Systemic BRAF/MEK inhibitor for Progressive/Relapsed Disease

| | | |
|---|---|---|
| Yes | 1 | 7.1 |
| No | 13 | 92.9 |

BRAF Mutation Status

| | | |
|---|---|---|
| BRAF mutant | 5 | 35.7 |
| BRAF wild type | 9 | 64.3 |

BRAF Status and Prior BRAFi/MEKi treatment use

| | | |
|---|---|---|
| BRAF mutant (no prior treatment) | 3 | 21.4 |
| BRAF mutant, prior adjuvant BRAFi/MEKi | 1 | 7.1 |
| treatment, no BRAFi/MEKi treatment for | | |
| metastatic disease | | |
| BRAF mutant, prior BRAFi/MEKi treatment | 1 | 7.1 |
| for metastatic disease, no adjuvant BRAFi/ | | |
| MEKi treatment | | |

No participants with M stage M1d; no participants had received previous localized treatment for brain metastasis

TABLE 5
Participants With Clinically Significant Adverse
Events by Lenvatinib CSAE Category and Preferred
Term (Incidence > 0%) (APaT Population)

| | n | % |
|---|---|---|
| Participants in population | 14 | |
| with one or more adverse events | 8 | (57.1) |
| with no adverse events | 6 | (42.9) |
| Cardiac Dysfunction | 1 | (7.1) |
| Cardiac failure | 1 | (7.1) |
| Hemorrhage | 1 | (7.1) |
| Haemoptysis | 1 | (7.1) |
| Hepatotoxicity | 1 | (7.1) |
| Gamma-glutamyltransferase increased | 1 | (7.1) |
| Hypertension | 6 | (42.9) |
| Hypothyroidism | 2 | (14.3) |
| Blood thyroid stimulating hormone increased | 1 | (7.1) |
| Hypothyroidism | 1 | (7.1) |
| Proteinuria | 1 | (7.1) |
| Renal Events | 1 | (7.1) |
| Blood creatinine increased | 1 | (7.1) |

TABLE 6
Summary of Participants with Drug related Adverse Events

| | MK-3475 400 mg Q6W + | |
|---|---|---|
| | MK-1308 25 mg Q6W + | |
| | Lenvatinib 20 mg QD | |
| | n | (%) |
| Subjects in APaT population | 14 | 100 |
| With drug-related AE | 12 | 85.7 |
| With Grade 3-5 drug-related AE | 4 | 28.6 |
| With dose interruptions due to drug-related | 3 | 21.4 |
| AE | | |
| With drug-related SAE | 2 | 14.3 |
| Who discontinued one or more drugs due | 1* | 7.1 |
| to a drug-related AE | | |
| Who died due to a drug-related\AE | 0 | 0 |
| Who discontinued all study treatment due | 0 | 0 |
| to a drug-related AE | | |

*Lenvatinib was discontinued after C3 due to drug related G3 embolism. Participant continued treatment with MK1308+ pembrolizumab
\hat{ }Details in slides 22 to 24 (pembrolizumab 400 mg Q6W + MK-1308 25 mg Q6W + Lenvatinib 20 mg QD)

TABLE 7
Particants With Serious Adverse Events (Incidence >
0%) By Decreasing Frequence of Preffered Term (APaT Population)

MK-3475 400 mg Q6W +
MK-1308 25 mg Q6W +
Lenvatinib 20 mg QD

| | n | (%) |
|---|---|---|
| Participants in population | 14 | |
| With one or more adverse events | 2 | (14.3) |
| With no adverse events | 12 | (85.7) |
| Embolism | 1 | (7.1) |
| Pancreatitis | 1 | (7.1) |

Every participant is counted a single time for each applicable row and column.
MedDRA V23.1 preferred terms “Neoplasm progression”, “Malignant neoplasm progression” and “Disease progression” not related to the drug are excluded.
Serious advese events up to 120 days of last dose are included.
Database Cutoff Date: 28 Oct. 2020.

TABLE 8
Participants with Grade 3-5 Adverse Events (Incidence >
0%) by Decreasing Frequence of Preferred Term (APaT Population)

| | MK-3475 400 mg Q6W + | |
|---|---|---|
| | MK-1308 25 mg Q6W + | |
| | Lenvatinib 20 mg QD | |
| | n | (%) |
| Participants in population | 14 | |
| With one or more adverse events | 4 | (28.6) |
| With no adverse events | 10 | (71.4) |
| Hypertension | 2 | (14.3) |
| Blood alkaline phosphatase increased | 1 | (7.1) |
| Embolism | 1 | (7.1) |
| Gamma-glutamyltransferase increased | 1 | (7.1) |

Every participant is counted a single time for each applicable row and column.
MedDRA V23.1 preferred terms “Neoplasm progression”, “Malignant neoplasm progression” and “Disease progression” not related to the drug are excluded.
Non-serious advese events up to 90 days of last dose and serious adverse events up to 120 days of last dose are included.
Grades are based on NCI CTCAE version 5.
Database Cutoff Date: 28 Oct. 2020.

TABLE 9
Adverse Event Summary (APaT Population)

| | | MK-3475 400 mg Q6W + | |
|---|---|---|---|
| | | MK-1308 25 mg Q6W + | |
| | | Lenvatinib 20 mg QD | |
| | | N | (%) |
| Participants in population | | 14 | |
| | With one or more adverse events | 12 | (85.7) |
| | With no adverse event | 2 | (14.3) |
| | With drug-related^{a} adverse events | 12 | (85.7) |
| | related to Pembrolizumab | 10 | (71.4) |
| | related to Quavonlimab | 10 | (71.4) |
| | related to Lenvatinib | 12 | (85.7) |
| | With toxicity grade 3-5 adverse events | 4 | (28.6) |
| | With toxicity grade 3-5 drug-related adverse events | 4 | (28.6) |
| | With serious adverse events | 2 | (14.3) |
| | With serious drug-related adverse events | 2 | (14.3) |
| | Who died | 0 | (0.0) |
| | Who died due to a drug-related adverse event | 0 | (0.0) |
| | With dose interruption^{b} due to an adverse event | 3 | (21.4) |
| | interruption of Pembrolizumab | 0 | (0.0) |
| | interruption of Quavonlimab | 0 | (0.0) |
| | interruption of Lenvatinib | 3 | (21.4) |
| | With dose reduction of Lenvatinib due to an adverse event | 0 | (0.0) |
| | Discontinued^{c} due to an adverse event | 1 | (7.1) |
| | discontinued Pembrolizumab | 0 | (0.0) |
| | discontinued Quavonlimab | 0 | (0.0) |
| | discontinued Lenvatinib | 1 | (7.1) |
| | Discontinued due to a drug-related adverse event | 1 | (7.1) |
| | Discontinued due to a serious adverse event | 1 | (7.1) |
| | Discontinued due to a serious drug-related adverse event | 1 | (7.1) |

^{a}Determined by the investigator to be related to the drug.
^{b}Dose interrption of any study medication
^{c}Study medication withdrawn of any study medication.
Non-serious adverse events up to 90 days following the last dose and serious adverse events up to 120 days followign the last dose are included.
MedDRA V23.1 preferred terms “Neoplasm progression”, “Malignant neoplasm progression” and “Disease progression” not related to the drug are excluded.
(Database cutoff Date: 28 Oct. 2020).

TABLE 10
Summary of Drug Exposure (APaT Population)

MK-3475 400 mg Q6W +
MK-1308 25 mg Q6W +
Lenvatinib 20 mg QD
(N = 14)

Number Days on Levantinib

| | |
|---|---|
| Mean | 31.8 |
| Median | 21.5 |
| SD | 26.50 |
| Range | 1.0 to 90.0 |

Number Days on Pembrolizumab

| | |
|---|---|
| Mean | 24.9 |
| Median | 1.0 |
| SD | 31.60 |
| Range | 1.0 to 85.0 |

Number Days on Quavonlimab

| | |
|---|---|
| Mean | 24.9 |
| Median | 1.0 |
| SD | 31.60 |
| Range | 1.0 to 85.0 |

Number of Adminstration of Lenvatinib

| | |
|---|---|
| Mean | 2.1 |
| Median | 1.5 |
| SD | 1.27 |
| Range | 1.0 to 5.0 |

TABLE 11
Participants with Adverse Events by System Organ Class
and Preferred Term (Incidence > 0%) (APaT Population)

| | MK-3475 400 mg Q6W + | |
|---|---|---|
| | MK-1308 25 mg Q6W + | |
| | Lenvatinib 20 mg QD | |
| | n | (%) |
| Participants in population | 14 | |
| With one or more adverse events | 12 | (85.7) |
| With no adverse events | 2 | (14.3) |
| Blood and lymphatic system disorders | 2 | (14.3) |
| Lymphopenia | 1 | (7.1) |
| Neutropenia | 1 | (7.1) |
| Thrombocytopenia | 1 | (7.1) |
| Cardiac disorders | 1 | (7.1) |
| Cardiac failure | 1 | (7.1) |
| Endocrine disorders | 1 | (7.1) |
| Hypothyroidism | 1 | (7.1) |
| Gastrointestinal disorders | 6 | (42.9) |
| Abdominal pain | 1 | (7.1) |
| Abdominal pain upper | 1 | (7.1) |
| Constipation | 2 | (14.3) |
| Diarrhoea | 2 | (14.3) |
| Dry Mouth | 1 | (7.1) |
| Dyspepsia | 1 | (7.1) |
| Lip discolouration | 1 | (7.1) |
| Odnophagia | 1 | (7.1) |
| Pancreatitis | 1 | (7.1) |
| Stomatitis | 1 | (7.1) |
| General disorders and adminsitration | 6 | (42.9) |
| site conditions | | |
| Asthenia | 3 | (21.4) |
| Chest discomfort | 1 | (7.1) |
| Chest pain | 1 | (7.1) |
| Chills | 1 | (7.1) |
| Fatigue | 1 | (7.1) |
| Hyperthermia | 1 | (7.1) |
| Mucosal inflammation | 2 | (14.3) |

TABLE 12
Participants with Adverse Events by System Organ Class
and Preferred Term (Incidence > 0%) (APaT Population)

| | MK-3475 400 mg Q6W + | |
|---|---|---|
| | MK-1308 25 mg Q6W + | |
| | Lenvatinib 20 mg QD | |
| | n | (%) |
| General Disorders and Administration | 6 | (42.9) |
| Site Conditions | | |
| Pyrexia | 2 | (14.3) |
| Infections and infestations | 2 | (14.3) |
| Candida infection | 1 | (7.1) |
| Folliculitis | 2 | (14.3) |
| Injury, poisoning and procedural | 1 | (7.1) |
| complications | | |
| Arthropod bite | 1 | (7.1) |
| Wound complication | 1 | (7.1) |
| Investigations | 4 | (28.6) |
| Blood alkaline phosphatase increased | 1 | (7.1) |
| Blood creatinine increased | 1 | (7.1) |
| Blood lactate dehydrogenase | 1 | (7.1) |
| Blood lactic acid increased | 1 | (7.1) |
| Blood thyroid stimulating hormone | 1 | (7.1) |
| increased | | |
| Blood triglycerides increased | 1 | (7.1) |
| C-reactive protein increased | 1 | (7.1) |
| Gamma-glutamyltransferase increased | 1 | (7.1) |
| Lipase increased | 2 | (14.3) |
| Weight decreased | 2 | (14.3) |
| Metabolism and nutrition disorders | 4 | (28.6) |
| Decreased appetite | 2 | (14.3) |
| Folate deficiency | 1 | (7.1) |
| Hypervolaemia | 1 | (7.1) |
| Hypoalbuminaemia | 1 | (7.1) |
| Hypokalaemia | 1 | (7.1) |
| Hypomagnesaemia | 2 | (14.3) |
| Hypophosphataemia | 2 | (14.3) |
| Musculoskeletal and connective tissue | 3 | (21.4) |
| disorders | | |
| Arthralgia | 1 | (7.1) |
| Back Pain | 1 | (7.1) |

TABLE 13
Particpants with Adverse Events by System Organ Class
and Preferred Term (Incidence > 0%) (APaT Population)

| | MK-3475 400 mg Q6W + | |
|---|---|---|
| | MK-1308 25 mg Q6W + | |
| | Lenvatinib 20 mg QD | |
| | n | (%) |
| Musculoskeletal and connective tissue | 3 | (21.4) |
| disorders | | |
| Myalgia | 1 | (7.1) |
| Neoplasms benign, malignant and | 1 | (7.1) |
| unspecified (incl cysts and polps) | | |
| Tumor pain | 1 | (7.1) |
| Nervous System Disorders | 4 | |
| Headache | 3 | (21.4) |
| Sciatica | 1 | (7.1) |
| Renal and Urinary Disorders | 1 | (7.1) |
| Proteinuria | 1 | (7.1) |
| Respiratory, thoracic and mediastinal | 7 | (50.0) |
| disorders | | |
| Cough | 1 | (7.1) |
| Dysphonia | 6 | (42.9) |
| Dyspnoea exertional | 1 | (7.1) |
| Haemoptysis | 1 | (7.1) |
| Oropharyngeal pain | 1 | (7.1) |
| Painful respiration | 1 | (7.1) |
| Productive cough | 1 | (7.1) |
| Skin and subcutaneous tissue disorders | 4 | (28.6) |
| Night sweats | 1 | (7.1) |
| Pruritius | 1 | (7.1) |
| Rash | 1 | (7.1) |
| Urticaria | 1 | (7.1) |
| Vascular Disorders | 8 | (57.1) |
| Deep vein htrombosis | 1 | (7.1) |
| Embolism | 1 | (7.1) |
| Hypertension | 6 | (42.9) |

TABLE 14
Participants with Adverse Events by System Organ Class
and Preferred Term (Incidence > 0%) (APaT Population)

MK-3475 400 mg Q6W +
MK-1308 25 mg Q6W +
Lenvatinib 20 mg QD
(N = 14)

| | n | (%) |
|---|---|---|
| Vascular Disorders | 8 | (57.1) |
| Hypotension | 1 | (7.1) |

Every participant is counted a single time for each applicable row and column
Non-serious adverse events up to 90 days of last dose and serious adverse events up to 120 days of last dose are included.
MedDRA V23.1 preferred terms “Neoplasm progression”, “Malignant neoplasm progression” and “Disease progression” not related to the drug are excluded.
Database Cutoff Date: 28 Oct. 2020

TABLE 15
Participants With Adverse Events Clinically Significant
Adverse Events (CSAE) by Maximum Toxicity Grade

| | pembrolizumab 400 mg Q6W + | |
|---|---|---|
| | MK-1308 25 mg Q6W + | |
| | Lenvatinib 20 mg QD | |
| | n | (%) |
| Participants in population | 14 | |
| with one or more Adverse Events | 8 | 57.1 |
| Grade 1 | 1 | 7.1 |
| Grade 2 | 4 | 28.6 |
| Grade 3 | 2 | 14.3 |
| Grade 4 | 1 | 7.1 |
| with no Adverse Events | 6 | 42.9 |
| Hypothyroidism | 1 | 7.1 |
| Grade 2 | 1 | 7.1 |
| Blood creatinine increased | 1 | 7.1 |
| Grade 2 | 1 | 7.1 |
| Blood thyroid stimulating hormone | 1 | 7.1 |
| increased | | |
| Grade 1 | 1 | 7.1 |
| Cardiac failure | 1 | 7.1 |
| Grade 1 | 1 | 7.1 |
| Gamma-glutamyltransferase increased | 1 | 7.1 |
| Grade 4 | 1 | 7.1 |
| Haemoptysis | 1 | 7.1 |
| Grade 1 | 1 | 7.1 |
| Hypertension | 6 | 42.9 |
| Grade 2 | 4 | 28.6 |
| Grade 3 | 2 | 14.3 |
| Proteinuria | 1 | 7.1 |
| Grade 2 | 1 | 7.1 |

### Objectives and Endpoints

A listing of objectives and endpoints for the clinical trial substudy are shown in the table below.

TABLE 16
Objectives and Endpoints

| | |
|---|---|
| Objectives | Endpoints |

Primary

| | |
|---|---|
| Safety Lead-in Phase: To assess the safety and | Dose-limiting toxicity (DLTs). |
| tolerability, and to establish an RP2D if applicable, | Adverse Events (AEs). |
| of treatment combinations that have not been | Study intervention discontinuations due |
| evaluated in a previous Phase 1 study. | to AEs. |
| Efficacy Phase: To assess the safety and tolerability | Adverse Events (AEs). |
| of each treatment arm based on the proportion of | Study intervention discontinuations due |
| participants with adverse events (AEs). | to AEs. |
| Efficacy Phase: To evaluate objective response rate | Objective Response (OR): Complete |
| (ORR) of each treatment arm as assessed by | response (CR) or partial response (PR). |
| Blinded Independent Central Review (BICR) per | |
| RECIST 1.1. | |

Secondary

| | |
|---|---|
| Efficacy Phase: To evaluate the duration of response | DOR: For participants who demonstrate |
| (DOR) as assessed by BICR per RECIST 1.1. | CR or PR, DOR is defined as the time |
| | from the first documented evidence of CR |
| | or PR until disease progression or death |
| | due to any cause, whichever occurs first. |
| Efficacy Phase: To evaluate progression-free | PFS: The time from the date of |
| survival (PFS) as assessed by BICR per | randomization to the date of the first |
| RECIST 1.1. | documented PD per RECIST 1.1 by |
| | BICR, or death from any cause, |
| | whichever occurs first. |
| Efficacy Phase: To evaluate overall survival (OS) | OS: The time from the date of |
| | randomization to the date of death. |
| Efficacy Phase: To evaluate clinical benefit rate | CBR: The percentage of participants who |
| (CBR) per RECIST 1.1 as assessed by BICR | have achieved stable disease (SD) ≥6 |
| | months or CR or PR based on assessments |
| | by BICR per RECIST 1.1. |

Tertiary/Exploratory

| | |
|---|---|
| Efficacy Phase: To evaluate the correlation of tumor | Tumor Size Change: Percent change of |
| size change with DOR, PFS, and OS. | the sum of target lesions from baseline |
| Efficacy Phase: To characterize the pharmacokinetic | Plasma concentration of each agent |
| (PK) profiles and anti-drug antibody (ADA) | ADA formation rate |
| formation for investigational agents. | |
| Efficacy Phase: To identify molecular (genomic, | Molecular (genomic, metabolic, and/or |
| metabolic, and/or proteomic) biomarkers that may | proteomic) determinants of response or |
| be indicative of clinical response/resistance, safety, | resistance to treatments, using blood |
| and/or the mechanism of action of study treatment | and/or tumor tissue. |
| combinations with pembrolizumab, lenvatinib and | |
| other investigational agents. | |

### Dosing

Initially, 3 Dose-Limiting Toxicity (DLT) evaluable participants will be enrolled in Dose Level 0. More than 3 to 6 participants may be enrolled to Dose Level 0 if needed to replace participants who are not evaluable for DLT or to ensure a thorough evaluation of the dose level during the Safety Lead-In Phase. An event will be considered a DLT if it occurs during the first treatment cycle, or 3 weeks from the first dose of study intervention and meets at least 1 of the criteria. Patients who experience a DLT will be allowed to remain on the substudy if they meet the following criteria: (1) the investigator believes it is appropriate for participant to remain on the substudy, and (2) the event has resolved and no longer meets the definition of DLT.

Dose recommendations for the next evaluable participants will depend on the observed number of DLTs in the first 3 DLT evaluable participants and the mTPI table until 10 participants are studied at Dose Level 0 with ≤4 of 10 participants experiencing a DLT. In the event Dose Level 0 is not tolerable, lower dose levels may be selected. As participants become evaluable for DLT assessment, the number of participants who are evaluable for DLT versus the number of participants who developed a DLT will be continuously assessed.

If enrollment expands to 10 participants for a dose level, and ≤4 of the 10 participants develop a DLT, then the dose confirmation will stop. If enrollment expands to 10 participants for a dose level and >4/10 participants develop a DLT, then the next lower dose may be expanded to further explore the dose-response relationship.

The investigator may attribute each toxicity event to lenvatinib alone, to the CTLA4 antagonist (ex. MK-1308) alone, to pembrolizumab alone, or to the combination, and follow the dose delay and restart criteria for the CTLA4 antagonist (ex. MK-1308) and pembrolizumab and for lenvatinib. Participants may not have any dose modifications of the CTLA4 antagonist (ex. MK-1308) or pembrolizumab in this investigational treatment arm. If toxicity attributed to the CTLA4 antagonist (ex. MK-1308) or pembrolizumab does not resolve or the criteria for resuming treatment are not met, the participant must be discontinued from the CTLA4 antagonist (ex. MK-1308) or pembrolizumab. Participants may have dose modifications of lenvatinib in this investigational treatment arm. If toxicity attributed to lenvatinib does not resolve or the criteria for resuming treatment are not met, the participant must be discontinued from lenvatinib. Refer to Dosing Tables below (Tables 6-9).

Holding of 1 agent and not the other agent is appropriate if, in the opinion of the investigator, the toxicity is clearly related to 1 of the study interventions. For example, in the combination arm, if the CTLA4 antagonist (ex. MK-1308) or pembrolizumab is held due to an adverse event attributed to that drug, lenvatinib may continue to be administered. Appropriate documentation is required regarding which drug the investigator is attributing to the adverse event. If, in the opinion of the investigator, the toxicity is related to the combination of 2 agents, then both drugs should be held according to recommended dose modifications. Exceptional circumstances to following the dose modification table may be considered after consultation with the Sponsor.

In case toxicity does not resolve to Grade 0 to 1 within 12 weeks after last treatment, the CTLA4 antagonist (ex. MK-1308) and/or pembrolizumab and/or lenvatinib should be discontinued after consultation with the Sponsor.

With investigator and Sponsor agreement, participants with a laboratory AE still at Grade 2 after 12 weeks may continue treatment in the trial only if asymptomatic and controlled.

After any Grade 4 drug-related AE, participants should not restart study intervention without consultation with the Sponsor. (Toxicity must have resolved to Grade 0 to 1 or baseline prior to restarting).

TABLE 17
Dosing Levels of Pembrolizumab, the CTLA4
antagonist (ex. MK-1308), and Lenvatinib

| | Dose Level 0 | Dose Level −1 | Dose Level −2 |
|---|---|---|---|
| Pembrolizumab | 400 mg | 400 mg | 400 mg |
| anti-CTLA4 antibody | 25 mg | 25 mg | 25 mg |
| Lenvatinib | 20 mg | 14 mg | 10 mg |

TABLE 18
Dose Modifications Guidelines for
Lenvatinib-Related Adverse Events

| | | |
|---|---|---|
| Treatment-Related | | |
| Toxicity^{a, b} | Management | Dose Adjustment |

Grade 1 or Tolerable Grade 2

| | |
|---|---|
| Continue treatment | No change |

Intolerable Grade 2^{c, d}, or Grade 3^{e, g}

| | | |
|---|---|---|
| First Occurrence | Interrupt lenvatinib until | Reduce lenvatinib dose |
| | resolved to Grade 0-1, or | to 14 mg once a day |
| | tolerable Grade 2 | (1-level reduction) |
| Second Occurrence | Interrupt lenvatinib until | Reduce lenvatinib dose |
| (same toxicity or | resolved to Grade 0-1, or | to 10 mg once a day |
| new toxicity) | tolerable Grade 2 | (1-level reduction) |
| Third Occurrence | Interrupt lenvatinib until | Reduce lenvatinib dose |
| (same toxicity or | resolved to Grade 0-1, or | to 8 mg once a day |
| new toxicity) | tolerable Grade 2 | (1-level reduction) |
| Fourth Occurrence | Interrupt lenvatinib | Discuss with sponsor |
| (same toxicity or | | |
| new toxicity) | | |

Grade 4f: Discontinue Study Intervention
^{a}An interruption of study intervention with lenvatinib for more than 28 days will require Sponsor approval before study intervention can be resumed.
^{b}Initiate optimal medical management for nausea, vomiting, hypertension, hypothyroidism and/or diarrhea prior to any lenvatinib interruption or dose reduction.
^{c}Applicable only to Grade 2 toxicities judged by the participant and/or physician to be intolerable.
^{d}Obese participants (BMI ≥ 30) with weight loss do not need to return to their baseline weight or within 10% of their baseline weight (ie, Grade 1 weight loss). These participants may restart study intervention at a lower dose once their weight remains stable for at least 1 week and they reach at least a BMI of 25. The new stable weight should be used as the new baseline for further dose reductions.
For asymptomatic laboratory abnormalities, such as Grade ≥ 3 elevantions of amylase and lipase that are not considered clinically relevant by the investigator, continuation of treatment should be discussed with sponsor.
fExcluding laboratory abnormalities judged to be non-life-threatening, in which case manage as Grade 3.
^{g}For Grade 3 thromboembolic event, permanently discontinue lenvatinib.

TABLE 19
Dose Modification and Toxicity Management Guidelines for Immune-
Related Adverse Events Associated with Pembrolizumab

| | Toxicity grade | Action with | Corticosteroid and/ | Monitoring |
|---|---|---|---|---|
| irAEs | (CTCAE V5.0) | Pembrolizumab | or other therapies | and follow-up |
| Pneumonitis | Grade 2 | Withold | Adminster corticosteroids | Monitor participants |
| | Recurrent | Permanently | (initial dose of 1-2 mg/lg | for signs and symptons of |
| | Grade 2, | discontinue | prednisone or equivalent | pneumonitis. Evaluate |
| | Grade 3, | | followed by taper) | participants with suspected |
| | or 4 | | Add prophylactic antibiotics | pneumonitis with radiographic |
| | | | for opportunistic infections | imaging and initiate |
| | | | | corticosteriod treatment. |
| Diarrhea/ | Grade 2 or 3 | Withold | Adminster corticosteroids | Monitor participants for |
| Colitis | Recurrent | Permanently | (initial dose of 1-2 mg/lg | signs andsymptons of |
| | Grade 3 or | discontinue | prednisone or equivalent | enterocolitis (ie, diarrhea, |
| | Grade 4 | | followed by taper) | abdominal pain, blood or |
| | | | | mucus in stool with or |
| | | | | without fever) and of bowel |
| | | | | perforation (eg, peritoneat |
| | | | | signs and ileus) |

General Instructions:
1. Severe and life-threatening irAEs shoud be treated with IV cortiscosteroids followed by oral steroids. Other immunosuppressive treatment should begin if the irAEs are not controlled by corticosteroids.
2. Pembrolizumab must be permanently discontinued if the irAE does not resolve or the corticosteroid dose is not ≤10 mg/day within 12 weeks of the last pembrolizumab treatment.
3. The corticosteroid taper should begin when the irAE is ≤ Grade 1 and continue at least 4 weeks.
4. If pembrolizumab has been withheld, pembrolizumab may resume after the irAE decreased to ≤ Grade 1 after corticosteroid taper.

TABLE 20
Pembrolizumab Infusion Reaction Dose Modification and Treatment Guidelines

| | | Premedication at |
|---|---|---|
| NCI CTCAE Grade | Treatment | Subsequent Dosing |
| Grade 1 | Increase monitoring of vital signs as | None |
| Mild reaction; | medically indicated until the participant is | |
| infusion interruption | deemed medically stable in the opinion of | |
| not indicated; | the investigator. | |
| intervention not | | |
| indicated. | | |
| Grade 2 | Stop Infusion | Participants may be |
| Requires therapy or | Additional appropriate medical therapy may | premedicated 1.5 hours |
| infusion interruption | include but is not limited to: | (±30 minutes) prior to |
| but responds promptly | IV fluids, Antihistamines, NSAIDs, | infusion of pembrolizumab |
| to symptomatic treatment | Acetaminophen, Narcotics. | with: |
| (eg, antihistamines, | Increase monitoring of vital signs as | Diphenhydramine 50 mg |
| NSAIDs, narcotics, | medically stable in the opinion of the | PO (or equivalent dose |
| IV fluids); prophylactic | investigator. | of antihistamine). |
| medications indicated | If symptons resolve within 1 hour of | Acetaminophen 500-1000 mg |
| for ≤24 hours. | stopping drug infusion, the infusion may be | PO (or equivalent dose of |
| | restarted at 50% of the original infusion rate | analgesic). |
| | (eg, from 100 mL/hr to 50 mL/hr). Otherwise | |
| | dosing will be held until symptons resolve | |
| | and the participant should be premedicated | |
| | for the next scheduled dose. | |
| | Participants who develop Grade 2 toxicitiy | |
| | despite adequate premedication should be | |
| | permanently discontinued from further study | |
| | intervention. | |

### Pembrolizumab Administration

Pembrolizumab will be administered as a 30-minute IV infusion on Day 1 of every treatment cycle if given Q3W or on Day 1 of every other treatment cycle if given Q6W. Sites should make every effort to target infusion timing to be as close to 30 minutes as possible. However, given the variability of infusion pumps from site to site, a window of −5 minutes and +10 minutes is permitted (i.e, infusion time is 30 minutes: −5 min/+10 min).

Pembrolizumab with MK-1308, a CTLA4 Antagonist

An ongoing, multicenter, multi-arm, open-label Phase 1b study (MK-1308-001) is evaluating MK-1308 (a CTLA4 antagonist) in combination with pembrolizumab in participants with solid tumors (NCT03179436). In dose escalation, MK-1308 was given at either 25 mg or 75 mg (Cohorts 1 and 2) IV Q3W as monotherapy × 1 cycle, in combination with pembrolizumab 200 mg Q3W×4 cycles, followed by pembrolizumab monotherapy. In dose confirmation, participants with 1 L advanced NSCLC were treated with MK-1308 at 25 mg Q3W (Arm A), 25 mg Q6W (Arm B), or 75 mg Q6W (Arm C), in combination with pembrolizumab 200 mg Q3W. Response was assessed Q9W.

This first-in-human study of MK-1308 was designed to assess the safety, tolerability, PK, and pharmacodynamics of escalating doses of MK-1308 when used in combination with pembrolizumab in participants with advanced/metastatic solid tumors refractory to conventional therapy, 1 L treatment-naïve NSCLC, and 2 L SCLC. It is reasonable to expect the PK of MK-1308 to be consistent with that of other humanized mAbs that typically have a low clearance and a limited volume of distribution. Using a population PK model of ipilimumab, distribution of exposures from the 25 mg MK-1308 fixed dose overlapped considerably with those obtained with the 0.3 mg/kg weight-based ipilimumab dose, and exposures from the 75 mg MK-1308 fixed dose overlapped considerably with those obtained with the 1.0 mg/kg weight-based ipilimumab dose [Feng, Y., et al 2014]. Similar to pembrolizumab, a fixed-dose regimen of MK-1308 is expected to reduce complexity in the logistical chain at treatment facilities and to reduce waste. The goal in introducing the lower doses of 25 mg MK-1308 at the schedule of either Q3W or Q6W was to determine if a similar response rate could be achieved at lower doses, which would be expected to result in a reduction in immune-related toxicities from CTLA-4 antagonism. The goal in evaluating the 75 mg MK-1308 dose at the Q6W interval was to achieve the expected response rates with similar acceptable toxicity and discontinuation rates based on the Phase 1 CheckMate-012 results in the NSCLC population [Hellmann, M. D., et al 2016]. Details regarding specific benefits and risks for participants participating in this clinical study may be found in the Investigator's Brochure (IB) and informed consent documents.

Participants with advanced melanoma that is refractory to PD-1/L1 are planned to be enrolled in this study in the efficacy expansion phase based on the following evidence: (1) improved response rates after ipilimumab/nivolumab in the Checkmate 067 study; (2) improved response rates after treatment with ipilimumab/pembrolizumab in the KEYNOTE-029 study and; (3) the results from a Merck single-institution collaborative study in a similar melanoma population [Wolchok, J. D., et al 2017] [Long, G. V., et al 2017] [Olsonm, D., et al 2018].

An interim analysis of MK-1308 safety, antitumor activity, pharmacokinetic (PK), and pharmacodynamic biomarker data was performed for the Dose Escalation and Dose Confirmation phases of the study based on a 7 Nov. 2018 database lock. The ORR by blinded independent central review (BICR) with confirmation for MK-1308 25 mg administered Q6W in the advanced 1 L NSCLC population (Arm B) was similar to the ORR for the 75 mg dose level given Q6W (33% versus 22%, respectively). The target rate for DLT was not reached in any of Cohorts 1, 2, or 3; however, a higher toxicity rate correlated with a higher MK-1308 dose among several different AE characteristics, including drug-related AEs, Grade 3 to 5 AEs, SAEs, and AEs leading to treatment discontinuation or modification. The Grade 3 to 5 AE rate was 29% in Cohort 1, 71% in Cohort 2, and 100% in Cohort 3. In the Dose Confirmation phase, a lower MK-1308 dose and a longer treatment interval had a more favorable toxicity profile (45% in Arm B, 48% in Arm A, 53% in Arm C, and 86% in Arm E [MK-1308 75 mg in combination with pembrolizumab 200 mg Q3W]). Time to first Grade 3 to 5 AE was more rapid at a higher MK-1308 dose and with more frequent dosing (7 months in Arm B, 5.2 months in Arm A, 5.6 months in Arm C, and 1.1 month in Arm E). The most common AEs were fatigue (24.9%), pruritus (24.4%), rash (23.5%), decreased appetite (22.5%), AST increased (19.2%), ALT increased (17.4%), diarrhea (16.4%), and hypothyroidism (16%). In totality, the efficacy, safety, and PK data led to the selection of 25 mg given Q6W as the RP2D for MK-1308 for use in combination with pembrolizumab.

Pembrolizumab with Lenvatinib

E7080-A001-111/KEYNOTE-146 is an ongoing multicohort Phase 2 study to assess the efficacy and safety of lenvatinib in combination with pembrolizumab in 6 types of biomarker-unselected metastatic solid tumors, including melanoma (excluding uveal melanoma), that have progressed after treatment with approved therapies or for which there are no standard effective therapies available. The study is ongoing but is no longer enrolling melanoma patients. Eligible patients are aged 18 years or older and have histologically confirmed nonuveal melanoma, 0 to 2 prior systemic anticancer regimens, and an ECOG score of 0 or 1. The primary endpoint is ORR at Week 24 based on iRECIST, as determined by investigator-read tumor assessments performed at baseline, Q6W until Week 24, and then Q9W thereafter. Secondary endpoints include Objective Response Rate (ORR), Duration of Response (DOR), Progression-free Survival (PFS), Overall Survival (OS), and safety and tolerability of the combination. All participants received lenvatinib 20 mg daily in combination with 200 mg pembrolizumab IV Q3W. At data cutoff (1 Mar. 2018), 21 metastatic melanoma patients were enrolled, and 38% of participants had 1 or more prior anticancer therapy.

For all enrolled participants (N=21), the ORR at Week 24 was 47.6% (95% CI: 25.7, 70.2) using iRECIST by investigator review. Of the 10 confirmed responses, 9 (42.9%) were PR, and 1 (4.8%) was CR. Stable disease was observed in 7 (33.3%) participants, and 3 (14.3%) experienced progressive disease. One participant had an unknown response. Median duration of objective response was 12.5 months (95% CI, 2.7 months, NE). Median PFS observed was 7.6 months (95% CI: 2.6 months, 15.8 months).

All participants experienced ≥1 treatment-related AE. There were no fatal treatment-related AEs. The most common any-grade treatment-related AEs were fatigue (52%), decreased appetite (48%), diarrhea (48%), hypertension (48%), dysphonia (43%), and nausea (43%). Dose reduction and interruption due to treatment-related AEs occurred in 13 (62%) and 10 (47.6%) participants, respectively. The safety profile of lenvatinib in combination with pembrolizumab appears manageable in patients with malignant melanoma and other tumor types and is consistent with each agent's safety profile when administered as monotherapy.

### Pembrolizumab, MK-1308, and Lenvatinib

The primary objective is to assess the safety and tolerability of investigational treatment combinations (MK-1308 in combination with pembrolizumab and lenvatinib) based on the proportion of participants with adverse events, and to evaluate the objective response rates (ORR) as assessed by blinded independently central review per Response Evaluation Criteria in Solid Tumors 1.1 (RECIST 1.1).

The secondary objective is to evaluate the duration of response (DOR) as assessed by BICR per RECIST 1.1. DOR is defined as the time from the first documented evidence of complete response (CR) or partial response (PR) until disease progression or death due to any cause, whichever occurs first.

The tertiary/exploratory objectives include the evaluation of progression-free survival (PFS) as assessed by BICR per RECIST 1.1. PFS is the time from the date of randomization/allocation to the date of the first documentation of disease progression or death from any cause, whichever occurs first. Additionally, this study will be used to evaluate overall survival (OS). OS is the time from the date of randomization/allocation to the date of death from any cause. ORR is evaluated as assessed by the investigator per RECIST 1.1 (and per RECIST 1.1 for Immune Based Therapeutics (iRECIST)). An objective of this study is to characterize the pharmacokinetic (PK) profile of each agent in the example, the development of circulating antidrug antibodies, of each agent, following administration, and to identify molecular (genomic, metabolic, and/or proteomic) biomarkers that may be indicative of clinical response/resistance, safety, and/or mechanism of action of pembrolizumab, MK-1308, and lenvatinib. The study will also be used to assess the score change from baseline and the time to true deterioration (TTD) in patient-reported outcomes (PRO) scores in global health status/quality of life (QoL) and physical functioning, to evaluate health status as assessed by the European Quality of Life 5-dimension 5-level (EQ-5D-5L) questionnaire to generate utility scores, and to assess the effects of therapy on tumor growth kinetics.

RECIST 1.1 is used by the BICR when assessing images for efficacy measures and by the local site when determining eligibility. Modified RECIST 1.1 for immune-based therapeutics (iRECIST) assessment has been developed and published by the RECIST Working Group, with input from leading experts from industry and academia, along with participation from the US Food and Drug Administration and the European Medicines Agency. The unidimensional measurement of target lesions, qualitative assessment of non-target lesions, and response categories are identical to RECIST 1.1, until progression is seen by RECIST 1.1. However, if a participant is clinically stable, additional imaging may be performed to confirm radiographic progression. iRECIST is used by investigators to assess tumor response and progression and to make treatment decisions as well as for exploratory efficacy analyses where specified.

Male/female participants with melanoma who are at least 18 years of age are enrolled in this study. A maximum of approximately 100 participants will be enrolled. The participant population is PD-1 refractory melanoma.

After a Screening Phase of up to 28 days, each participant will receive study intervention for approximately 2 years or until disease progression is radiographically documented per RECIST 1.1 by the investigator, unacceptable AEs, withdrawal of consent or death, intercurrent illness that prevents further administration of study intervention, investigator's decision to discontinue the participant, noncompliance with study intervention or procedure requirements, or administrative reasons requiring cessation of study intervention, whichever occurs first. Participants who attain an investigator-determined confirmed complete response (CR) may consider stopping study intervention after at least 24 weeks of study intervention has been administered. In addition, if a confirmed CR per RECIST 1.1 is attained, at least 2 additional doses of study intervention must be received after CR is first documented.

Participants will be permitted to continue study intervention beyond RECIST 1.1-defined disease progression as long as the treating investigator considers that the participant may experience clinical benefit with continued intervention, and the participant is tolerating study intervention as per iRECIST (RECIST 1.1 for immune-based therapeutics). Treatment beyond disease progression per iRECIST may be permitted upon Sponsor consultation and approval.

After the End of Treatment (EOT), each participant will be followed for the occurrence of AEs and spontaneously reported pregnancy.

Participants who discontinue due to radiographic disease progression will move into posttreatment Safety and Survival Follow-up. Participants who discontinue for reasons other than radiographic disease progression will have posttreatment Safety and Follow-up imaging for disease status until disease progression is documented radiographically per RECIST 1.1 by the investigator, a nonstudy cancer treatment is initiated, consent is withdrawn, pregnancy, death, or becoming lost to follow-up. All participants will be followed for overall survival (OS) until death, withdrawal of consent, or the end of this study.

This study will include participants with PD-1 refractory melanoma. PD-1 refractory melanoma must have progressed on treatment with an anti-PD-1/L1 mAb administered either as monotherapy, or in combination with other checkpoint inhibitors or other therapies. Participants must have received at least 2 doses of an approved anti-PD-1/L1 mAb and have demonstrated disease progression after PD-1/L1 as defined by RECIST 1.1. No approved therapies exist in this setting and therefore, this population is considered as having a high need.

This study will consist of 1 or more investigational treatment arms evaluating pembrolizumab based/non-pembrolizumab-based combinations. If more than 1 investigational treatment arm is open at any given time, participants will be randomly assigned to 1 of the investigational treatment arms open for enrollment.

This study will only consist of an Efficacy Phase once the RP2D of a combination has been established in a separate Phase 1 study. However, when the RP2D of the combination has not previously been established (i.e, pembrolizumab combined with 2 or more investigational agents), this study will have 2 phases: an initial Safety Lead-in Phase, in which the preliminary RP2D of the combination will be established, followed by an Efficacy Phase.

### Efficacy Endpoints

The primary efficacy objective of this study is to evaluate the antitumor effect of various investigational agents with or without pembrolizumab in participants with PD-1 refractory melanoma.

This study will use objective response as the primary efficacy endpoint. Objective response rate is defined as the proportion of participants who have best response as CR or PR. Responses are based on BICR using RECIST 1.1 (modified to follow a maximum of 10 target lesions and a maximum of 5 target lesions per organ). Objective response rate is an appropriate endpoint to evaluate the antitumor activity of investigational treatment arms.

A treatment effect measured by ORR can support accelerated approval, support traditional approval, or represent direct clinical benefit based on the specific disease, context of use, magnitude of the effect, number of CRs, durability of response, disease setting, location of the tumors, available therapy, and risk-benefit relationship.

This study will use DOR as a secondary efficacy endpoint. Duration of response is defined as the time from the earliest date of qualifying response until the earliest date of disease progression or death from any cause, whichever comes first. Duration of response per RECIST 1.1, modified to follow a maximum of 10 target lesions and a maximum of 5 target lesions per organ, assessed by BICR will serve as an additional measure of efficacy and is a commonly accepted endpoint by both regulatory authorities and the oncology community.

This study will use PFS as an exploratory efficacy endpoint. Progression-free survival is defined as the time from date of randomization/allocation until the first date of disease progression or death from any cause, whichever comes first, based on RECIST 1.1 criteria as assessed by BICR. Images will be read by a central imaging vendor blinded to treatment assignment to minimize bias in the response assessments. Progression-free survival can reflect tumor growth and be assessed before the determination of a survival benefit. Its determination is not confounded by subsequent therapy. Treatment effect measured by PFS can be a surrogate endpoint to support accelerated approval, a surrogate endpoint to support traditional approval, or it can represent direct clinical benefit based on the specific disease, context of use, magnitude of the effect, the disease setting, location of metastatic sites, available therapy, the risk-benefit relationship, and the clinical consequences of delaying or preventing progression in key disease sites (e.g., delay of new lesions in the brain or spine) or delaying administration of more toxic therapies. This study will also evaluate ORR as assessed by investigator per RECIST 1.1 as an exploratory endpoint.

Additionally, this study will use OS as an exploratory efficacy endpoint. Overall survival has been recognized as the gold standard for the demonstration of superiority of a new antineoplastic therapy in randomized clinical studies. Overall survival in this study is defined as the time from date of randomization/allocation to death from any cause.

### Inclusion Criteria

Participants are eligible to be included in the study only if all the of following apply:
1. Has histologically or cytologically confirmed melanoma.
2. Has unresectable Stage III or Stage IV melanoma, per AJCC 8th Edition Staging Criteria (see Appendix 9), not amenable to local therapy.
3. Has the presence of at least 1 measurable lesion by CT or MRI per RECIST 1.1 as confirmed by BICR.
   a. Cutaneous lesions and other superficial lesions are not considered measurable lesions but may be considered as nontarget lesions.
   b. If participants have only 1 measurable lesion per RECIST 1.1, the biopsy specimen should be obtained from the nontarget lesion or archival tissue. If biopsy specimen was obtained from a lone target lesion, a repeat screening CT must be obtained post biopsy and measurable disease confirmed by BICR.
   c. Lesions that are in an area that has been previously irradiated should not be considered measurable unless there has been documented growth of the lesions since the completion of radiation.
4. Has progressed on treatment with an anti-PD-1/L1 mAb administered either as monotherapy, or in combination with other checkpoint inhibitors or other therapies. PD-1 treatment progression is defined by meeting all of the following criteria:
   a. Received at least 2 doses of an approved anti-PD-1/L1 mAb.
   b. Demonstrated disease progression after PD-1/L1 as defined by RECIST 1.1. The initial evidence of disease progression is to be confirmed by a second assessment no less than 4 weeks from the date of the first documented progressive disease, in the absence of rapid clinical progression. This determination is made by the investigator. Once progressive disease is confirmed per iRECIST by the investigator, the initial date of progressive disease documentation will be considered the date of disease progression.
   c. Progressive disease has been documented within 12 weeks from the last dose of anti-PD-1/L1 mAb.
   d. Participants who receive anti-PD-1 therapy as adjuvant treatment following complete resection of Stage III or IV melanoma and have disease recurrence (unresectable loco-regional disease or distant metastases) while on active treatment or within 6 months of stopping anti-PD-1. For these participants, the following applies: 1) a second assessment to confirm disease progression beyond recurrence is not required; and 2) they must have received at least 2 prior doses of anti-PD-1/L1 mAb. This participant population may be limited to approximately 20% in each investigational treatment arm.
   e. Prior therapy with CTLA-4 inhibitors will not be allowed.
5. Has submitted prestudy imaging. Note: The site's study team must have reviewed prestudy images that are of diagnostic quality from at least 3 dates to determine that radiographic progression has occurred per RECIST 1.1/iRECIST following initiation of an anti-PD-1/L1 agent. The CIV must have received these scans and have confirmed that they are of acceptable diagnostic quality prior to treatment randomization/allocation in this study for a retrospective analysis of Inclusion Criterion 4: participants must be refractory to anti-PD-1/L1 agents. The CIV will not be confirming this eligibility criterion prior to treatment randomization/allocation.
6. Has not received more than 3 lines of therapy for their advanced melanoma.
7. Has provided a tumor biopsy.
   a. Participants must submit tumor sample during Screening for confirmation of adequacy of tumor tissue at a central pathology laboratory. Participants who do not submit a tumor tissue sample will not be randomized/allocated.
   b. Tumor sample should be freshly obtained (strongly preferred). In cases where newly obtained tissue is not possible to provide; an archival sample may be acceptable after discussion with the Sponsor. Only archival samples that have been obtained after progression on a PD-1/L1 agent will be accepted.
   c. If a fresh tissue sample is submitted, it is preferred that the tumor biopsy is not obtained from a lone target lesion. If the biopsy specimen was obtained from a lone target lesion, a repeat screening CT must be obtained post-biopsy and measurable disease confirmed by BICR.
8. Male Participants—Male participants are eligible to participate if they agree to the following during the intervention period and for at least 30 days after the last dose of lenvatinib:
   a. Be abstinent from heterosexual intercourse as their preferred and usual lifestyle (abstinent on a long term and persistent basis) and agree to remain abstinent, or
   b. Must agree to use contraception unless confirmed to be azoospermic (vasectomized or secondary to medical cause) as below:
      i. Agree to use a male condom plus partner use of an additional contraceptive method when having penile-vaginal intercourse with a women of childbearing potential (WOCBP) who is not currently pregnant. Note: Men with a pregnant or breastfeeding partner must agree to remain abstinent from penile-vaginal intercourse or use a male condom during each episode of penile-vaginal penetration.
9. A female participant is eligible to participate if she is not pregnant or breastfeeding, and at least 1 of the following conditions applies:
   a. Is not a WOCBP, or
   b. Is a WOCBP and using a contraceptive method that is highly effective (with a failure rate of <1% per year), with low user dependency, or be abstinent from heterosexual intercourse as their preferred and usual lifestyle (abstinent on a long term and persistent basis), as described in Appendix 5 during the intervention period and for at least 120 days post pembrolizumab or 30 days post lenvatinib, whichever occurs last. The investigator should evaluate the potential for contraceptive method failure (i.e, noncompliance, recently initiated) in relationship to the first dose of study intervention
   A WOCBP must have a negative highly sensitive pregnancy test (urine or serum as required by local regulations) within 24 hours before the first dose of study intervention.
   If a urine test cannot be confirmed as negative (e.g., an ambiguous result), a serum pregnancy test is required. In such cases, the participant must be excluded from participation if the serum pregnancy result is positive.
   The investigator is responsible for review of medical history, menstrual history, and recent sexual activity to decrease the risk for inclusion of a woman with an early undetected pregnancy.

### Exclusion Criteria

The participant must be excluded from the study if the participant:

### Medical Conditions

1. Has a diagnosis of immunodeficiency or is receiving chronic systemic steroid therapy (in dosing exceeding 10 mg daily of prednisone equivalent) or any other form of immunosuppressive therapy within 7 days before the first dose of study intervention. Participants with asthma that require intermittent use of bronchodilators, inhaled steroids, or local steroid injections would not be excluded from the study.
2. Has a known additional malignancy that is progressing or requires active treatment within the past 2 years. Exceptions to the secondary malignancy exclusion include basal cell carcinoma of the skin, squamous cell carcinoma of the skin, new nonulcerated primary melanoma <1 mm in depth with no nodal involvement, Grade 1 follicular lymphoma or carcinoma in situ (e.g., breast carcinoma, cervical cancer in situ) that have undergone potentially curative therapy.
3. Has known active CNS metastases and/or carcinomatous meningitis. Participants with previously treated CNS metastases may participate provided they are stable (without evidence of progression by imaging prior to the first dose of study intervention as evidenced by 2 scans at least 4 weeks apart providing stability, and any neurologic symptoms have returned to baseline), have no evidence of new or enlarging brain metastases confirmed by repeat imaging, and have not required steroids for at least 14 days before study intervention. The second scan showing stability may be used as baseline scan if acquired within the Screening Phase.
   Note: Participants with asymptomatic previously untreated brain metastases may participate provided there are ≤3 total lesions in the brain and their longest diameter is <1 cm. Stability of these lesions does not need to be confirmed by repeat imaging. Baseline MRI brain scan will be obtained for all participants. Brain CT scan should only be used when MRI is contraindicated. The second brain MRI showing stability may be used as baseline scan if acquired within the Screening Phase.
4. Has ocular or mucosal melanoma.
5. Has known hypersensitivity to active substances or any of their excipients including previous clinically significant hypersensitivity reaction to treatment with another mAb. For a list of excipients, refer to the respective IB.
6. Has an active autoimmune disease that has required systemic treatment in the past 2 years (i.e, with use of disease modifying agents, corticosteroids, or immunosuppressive drugs). Replacement therapy (e.g., thyroxine, insulin, or physiologic corticosteroid replacement therapy for adrenal or pituitary insufficiency) is not considered a form of systemic treatment and is allowed.
7. Has an active infection requiring systemic therapy.
8. Has known history of human immunodeficiency virus (HIV; HIV 1/2 antibodies). No testing of HIV is required unless mandated by local health authority (see Appendix 11 for country-specific requirements).
9. Has known history of hepatitis B (defined as HBsAg reactive) or known hepatitis C virus (defined as HCV RNA [qualitative] is detected) infection. Note: No testing for hepatitis B and hepatitis C is required unless mandated by local health authority (see Appendix 11 for country-specific requirements).
10. Has a history of (noninfectious) pneumonitis that required steroids or current pneumonitis.
11. Has a history of active tuberculosis (TB; Bacillus tuberculosis). (See Appendix 11 for country-specific requirements).
12. A WOCBP who has a positive urine pregnancy test within 24 hours prior to randomization or treatment allocation (see Appendix 5). If the urine test is positive or cannot be confirmed as negative, a serum pregnancy test will be required. Note: In the event that more than 24 hours have elapsed between the Screening pregnancy test and the first dose of study intervention, another pregnancy test (urine or serum) must be performed and must be negative in order for the participant to start receiving study intervention.

### Prior/Concomitant Therapy

13. Has received prior systemic anticancer therapy including investigational agents within 4 weeks prior to randomization/allocation. Note: Participants must have recovered from all AEs due to previous therapies to ≤Grade 1 or baseline. Participants with ≤Grade 2 neuropathy and/or ≤Grade 2 endocrinopathy may be eligible. Note: If participant received major surgery, they must have recovered adequately from the toxicity and/or complications from the intervention prior to starting study intervention.
14. Has received prior radiotherapy within 2 weeks of first dose of study intervention. Participants must have recovered from all radiation-related toxicities, not require corticosteroids, and not have had radiation pneumonitis.
15. Has had major surgery (<3 weeks prior to first dose of study intervention) Note: If the participant received major surgery, they must have recovered adequately from the toxicity and/or complications from the intervention prior to first dose of study intervention. Adequate wound healing after major surgery must be assessed clinically, independent of time elapsed for eligibility.
16. Has received a live vaccine within 30 days before the first dose of study intervention. Examples of live vaccines include, but are not limited to, the following: measles, mumps, rubella, varicella/zoster (chicken pox), yellow fever, rabies, BCG, and typhoid vaccine. Seasonal influenza vaccines for injection are generally killed virus vaccines and are allowed; however, intranasal influenza vaccines (e.g., FluMist®) are live attenuated vaccines and are not allowed.

### Prior/Concurrent Clinical Study Experience

17. Is currently participating in or has participated in a study of an investigational agent or has used an investigational device within 4 weeks prior to the first dose of study intervention. Note: Participants who have entered the Follow-up phase of an investigational study may participate if it has been 4 weeks since the last dose of the previous investigational agent.

### Other Exclusions

18. Has a history or has current evidence of any condition, therapy, or laboratory abnormality that might confound the results of the study, interfere with the participation for the full duration of the study, or is not in the best interest of the participant to participate, in the opinion of the treating investigator.
19. Is pregnant or breastfeeding or expecting to conceive or father children within the projected duration of the study, starting with the Screening visit through 120 days after the last dose of study intervention.
20. Has had an allogeneic tissue/solid organ transplant.
21. Has a pre-existing Grade ≥3 gastrointestinal or nongastrointestinal fistula.
22. Has radiographic evidence of encasement of invasion of a major blood vessel, or of intratumoral cavitation. Note: the degree of proximity to major blood vessels should be considered because of the potential risk of severe hemorrhage associated with tumor shrinkage/necrosis following lenvatinib therapy.
23. Has clinically significant hemoptysis or tumor bleeding within 2 weeks prior to the first dose of study intervention.
24. Has clinically significant cardiovascular disease within 12 months from first dose of study intervention, including New York Heart Association Class III or IV congestive heart failure, unstable angina, myocardial infarction, cerebral vascular accident, or cardiac arrhythmia associated with hemodynamic instability. Note: Medically controlled arrhythmia would be permitted.
25. Has urine protein ≥1 g/24-hour. Note: Participants having >1+ proteinuria on urinalysis will undergo 24-hour urine collection for quantitative assessment of proteinuria.
26. Has prolongation of QTc interval (calculated using Fridericia's formula) to >480 msec.
27. Has LVEF below the institutional normal range as determined by MUGA or echocardiogram.
28. Has presence of gastrointestinal condition including malabsorption, gastrointestinal anastomosis, or any other condition that might affect the absorption of lenvatinib.
29. Has a known psychiatric or substance abuse disorder that would interfere with cooperation with the requirements of the study.

### Dose Limiting Toxicity

Dose-limiting toxicities will be defined from toxicities observed during the first cycle of the Safety Lead-in Phase. The occurrence of any of the following toxicities will be considered a DLT unless the investigator assessment determines the toxicity to be clearly not related to the study intervention:
Grade 4 nonhematologic toxicity (not laboratory)
Grade 4 hematologic toxicity lasting >=7 days, except
   Grade 4 platelet count decreased of any duration
   Grade 3 platelet count decreased if associated with bleeding
   Grade 4 lymphopenia lasting ≥21 days
Any nonhematologic AE>=Grade 3 (not laboratory) lasting >3 days despite optimal supportive care
Any Grade 3 or Grade 4 clinically significant nonhematologic laboratory abnormality, if:
   medical intervention is required to treat the participant, or
   the abnormality leads to hospitalization, or
   the abnormality persists for >1 week
any febrile neutropenia Grade 3 or Grade 4
Any treatment related AE that causes the participant to discontinue study intervention during the DLT window
Any Grade 5 toxicity
Any treatment-related toxicity that causes a >2 week delay in initiation of Cycle 3

### Assessment at Screening and Prior to RECIST 1.1 Progression

Until radiographic disease progression based on RECIST 1.1, there is no distinct iRECIST assessment.

### Assessment and Decision at RECIST 1.1 Progression

For participants who show evidence of radiological progressive disease by RECIST 1.1 as determined by the investigator, the investigator will decide whether to continue a participant on study intervention until repeat imaging is obtained (using iRECIST for participant management). This decision by the investigator should be based on the participant's overall clinical condition.

Clinical stability is defined as the following:
Absence of symptoms and signs indicating clinically significant progression of disease
No decline in ECOG performance status
No requirements for intensified management, including increased analgesia, radiation, or other palliative care

Any participant deemed clinically unstable should be discontinued from study intervention at site-assessed first radiologic evidence of progressive disease and is not required to have repeat tumor imaging for confirmation of progressive disease by iRECIST.

If the investigator decides to continue treatment, the participant may continue to receive study intervention and the tumor assessment should be repeated 4 to 8 weeks later to confirm progressive disease by iRECIST, per investigator assessment. Images should continue to be sent in to the central imaging vendor for potential retrospective BICR.

Tumor flare may manifest as any factor causing radiographic progression per RECIST 1.1, including:
Increase in the sum of diameters of target lesion(s) identified at baseline to ≥20% and
≥5 mm from nadir
Note: The iRECIST publication uses the terminology “sum of measurements,” but “sum of diameters” will be used in this protocol, consistent with the original RECIST 1.1 terminology.
Unequivocal progression of nontarget lesion(s) identified at baseline
Development of new lesion(s)

iRECIST defines new response categories, including iUPD (unconfirmed progressive disease) and iCPD (confirmed progressive disease). For purposes of iRECIST assessment, the first visit showing progression according to RECIST 1.1 will be assigned a visit (overall) response of iUPD, regardless of which factors caused the progression.

At this visit, target and nontarget lesions identified at baseline by RECIST 1.1 will be assessed as usual.

New lesions will be classified as measurable or non-measurable using the same size thresholds and rules as for baseline lesion assessment in RECIST 1.1. From measurable new lesions, up to 5 lesions total (up to 2 per organ), may be selected as New Lesions—Target. The sum of diameters of these lesions will be calculated and kept distinct from the sum of diameters for target lesions at baseline. All other new lesions will be followed qualitatively as New Lesions—Nontarget.

### Assessment at the Confirmatory Imaging

On the confirmatory imaging, the participant will be classified as progression confirmed (with an overall response of iCPD), or as showing persistent unconfirmed progression (with an overall response of iUPD), or as showing disease stability or response (iSD/iPR/iCR).

### Confirmation of Progression

Progression is considered confirmed, and the overall response will be iCPD, if ANY of the following occurs:
Any of the factors that were the basis for the iUPD at the previous visit show worsening
   For target lesions, worsening is a further increase in the sum of diameters of ≥5 mm, compared to any prior iUPD time point
   For nontarget lesions, worsening is any significant growth in lesions overall, compared to a prior iUPD time point; this does not have to meet the “unequivocal” standard of RECIST 1.1
   For new lesions, worsening is any of these:
      An increase in the new lesion sum of diameters by ≥5 mm from a prior iUPD time point
      Visible growth of new nontarget lesions
      The appearance of additional new lesions
Any new factor appears that would have triggered progressive disease by RECIST 1.1
   Persistent iUPD

Progression is considered not confirmed, and the overall response remains iUPD, if:
None of the progression-confirming factors identified above occurs AND
The target lesion sum of diameters (initial target lesions) remains above the initial PD threshold (by RECIST 1.1)

Additional imaging for confirmation should be scheduled 4 to 8 weeks from the imaging on which iUPD is seen. This may correspond to the next visit in the original visit schedule. The assessment of the subsequent confirmation imaging proceeds in an identical manner, with possible outcomes of iCPD, iUPD, and iSD/iPR/iCR.

Resolution of iUPD

Progression is considered not confirmed, and the overall response becomes iSD/iPR/iCR, if:
None of the progression-confirming factors identified above occurs, AND
The target lesion sum of diameters (initial target lesions) is not above the initial progressive disease threshold.

The response is classified as iSD or iPR (depending on the sum of diameters of the target lesions), or iCR if all lesions resolve.

In this case, the initial iUPD is considered to be pseudo-progression, and the level of suspicion for progression is “reset.” This means that the next visit that shows radiographic progression, whenever it occurs, is again classified as iUPD by iRECIST, and the confirmation process is repeated before a response of iCPD can be assigned.

### Management Following the Confirmatory Imaging

If repeat imaging does not confirm progressive disease per iRECIST, as assessed by the investigator, and the participant continues to be clinically stable, study intervention may continue and follow the regular imaging schedule. If progressive disease is confirmed, participants will be discontinued from study intervention.

Detection of Progression at Visits after Pseudo-Progression Resolves

After resolution of pseudo-progression (i.e., achievement of iSD/iPR/iCR), iUPD is indicated by any of the following events:
Target lesions
   Sum of diameters reaches the progressive disease threshold (≥20% and ≥5 mm increase from nadir) either for the first time, or after resolution of previous pseudo-progression. The nadir is always the smallest sum of diameters seen during the entire substudy, either before or after an instance of pseudo-progression.
Nontarget lesions
   If nontarget lesions have never shown unequivocal progression, their doing so for the first-time results in iUPD.
   If nontarget lesions have shown previous unequivocal progression, and this progression has not resolved, iUPD results from any significant further growth of non-target lesions, taken as a whole.
New lesions
   New lesions appear for the first time
   Additional new lesions appear
   Previously identified new target lesions show an increase of ≥5 mm in the new lesion sum of diameters, from the nadir value of that sum
   Previously identified nontarget lesions show any significant growth

If any of the events above occur, the overall response for that visit is iUPD, and the iUPD evaluation process (see Assessment at the Confirmatory Imaging above) is repeated.

Progression must be confirmed before iCPD can occur. The decision process is identical to the iUPD confirmation process for the initial progressive disease, with 1 exception: If new lesions occurred at a prior instance of iUPD, and at the confirmatory imaging the burden of new lesions has increased from its smallest value (for new target lesions, the sum of diameters is ≥5 mm increased from its nadir), then iUPD cannot resolve to iSD or iPR. It will remain iUPD until either a decrease in the new lesion burden allows resolution to iSD or iPR, or until a confirmatory factor causes iCPD. Additional details about iRECIST are provided in the iRECIST publication [Seymour, L., et al 2017].

### AJCC 8^{th} Edition Staging Criteria and Overall Staging Table

The AJCC has designated staging by TNM classification to define melanoma. The following staging tables (Tables 21-24) adapted from AJCC 8th edition. Refer to AJCC guidelines for more information [Gershenwald, J. E., et al 2017]. ECOG performance status is listed in Table 25.

TABLE 21
Melanoma T Category Definition

| T Stage | T Stage Definition (thickness and ulceration) |
|---|---|
| TX | Primary tumor thickness cannot be assessed |
| | (ulceration status not applicable) |
| T0 | No evidence of primary tumor (ulceration status not applicable) |
| Tis | Melanoma in situ (ulceration status not applicable) |
| T1 | ≤1.0 mm (ulceration status unknown or unspecified) |
| T1a | <0.8 mm without ulceration |
| T1b | <0.8 mm with ulceration or 0.8-1.0 mm with or without |
| | ulceration |
| T2 | >1.0-2.0 mm (ulceration status unknown or unspecified) |
| T2a | >1.0-2.0 mm without ulceration |
| T2b | >1.0-2.0 mm with ulceration |
| T3 | >2.0-4.0 mm (ulceration status unknown or unspecified) |
| T3a | >2.0-4.0 mm without ulceration |
| T3b | >2.0-4.0 mm with ulceration |
| T4 | >4.0 mm (ulceration status unknown or unspecified) |
| T4a | >4.0 mm without ulceration |
| T4b | >4.0 mm with ulceration |

Abbreviations:
T = primary tumor.

TABLE 22
Melanoma N Category Definition

| N | Number of Tumor-involved | Presence of In-transit, Satellite, |
|---|---|---|
| Category | Regional Lymph Nodes | and/or Microsatellite Metastases |
| NX | Regional Nodes not assessed (exception: | No |
| | pathological N category not required for T1 | |
| | melanomas, use clinical N information) | |
| N0 | No regional metastases detected | No |
| N1 | One tumor-involved node or any number of in- | |
| | transit, satellite, and/or microsatellite | |
| | metastases with no tumor-involved nodes | |
| N1a | One clinically occult (detected by SLN biopsy) | No |
| N1b | One clinically detected | No |
| N1c | No regional lymph node disease | Yes |
| N2 | 2 or 3 tumor-involved nodes or any number of | |
| | in-transit, satellite, and/or micro-satellite | |
| | metastases with 1 tumor-involved node | |
| N2a | 2 or 3 clinically occult (detected by SLN biopsy) | No |
| N2b | 2 or 3, at least one of which was clinically detected | No |
| N2c | One clinically occult or clinically detected | Yes |
| N3 | Four or more tumor-involved nodes or any number | |
| | of in-transit, satellite, and/or microsatellite | |
| | metastases with 2 or more tumor-involved nodes, or | |
| | any number of matted nodes without or with in- | |
| | transit, satellite, and/or microsatellite metastases. | |
| N3a | 4 or more clinically occult (detected by SLN biopsy) | No |
| N3b | 4 or more, at least one of which was clinically | No |
| | detected, or the presence of any number of matted | |
| | nodes | |
| N3c | 2 or more clinically occult or clinically detected | Yes |
| | and/or presence of any number of matted nodes. | |

Abbreviations:
N = regional lymph node; SLN = sentinel lymph node.

TABLE 23
Melanoma M Category Definition

| M Category | Anatomic Site | Lactate Dehydrogenase Level |
|---|---|---|
| M0 | No evidence of distant metastasis | Not applicable |
| M1 | Evidence of distant metastasis | See below |
| M1a | Distant metastasis to skin, soft tissue | Not recorded or unspecified |
| | including muscle, and/or nonregional | |
| | lymph node | |
| M1a(0) | | Not elevated |
| M1a(1) | | Elevated |
| M1b | Distant metastasis to lung with or without | Not recorded or unspecified |
| | M1a sites of disease | |
| M1b(0) | | Not elevated |
| M1b(1) | | Elevated |
| M1c | Distant metastasis to noncentral nervous | Not recorded or unspecified |
| | system visceral sites with or without M1a | |
| | or M1b sites of disease | |
| M1c(0) | | Not elevated |
| M1c(1) | | Elevated |
| Mid | Distant metastasis to CNS with or without | Not recorded or unspecified |
| | M1a, M1b, or M1c sites of disease | |
| M1d(0) | | Not elevated |
| M1d(1) | | Elevated |

Abbreviations:
CNS = central nervous system; M = distant metastasis.

TABLE 24
AJCC Pathological (pTNM) Staging Groups
Staging (AJCC 8th edition)

| | | | |
|---|---|---|---|
| 0 | Tis | N0 | M0 |
| IA | T1a | N0 | M0 |
| IA | T1b | N0 | M0 |
| IB | T2a | N0 | M0 |
| IIA | T2b | N0 | M0 |
| IIA | T3a | N0 | M0 |
| IIB | T3b | N0 | M0 |
| IIB | T4a | N0 | M0 |
| IIC | T4b | N0 | M0 |
| IIIB | T0 | N1b, N1c | M0 |
| IIIC | T0 | N2b, N2c, N3b, N3c | M0 |
| IIIA | T1a/b-T2a | N1a or N2a | M0 |
| IIIB | T1a/b-t2a | N1b/c orN2b | M0 |
| IIIB | T2b/T3a | N1a-N2b | M0 |
| IIIC | T1a-T3a | N2c or N3a/b/c | M0 |
| IIIC | T3b/T4a | Any N ≥ N1 | M0 |
| IIIC | T4b | N1a-N2c | M0 |
| IIID | T4b | N3a/b/c | M0 |
| IV | Any T, Tis | Any N | M1 |

Abbreviations:
M0 = No evidence of distant metastases; N0 = No regional metastasis detected including no tumor-involved nodes and no in-transit, satellite, and/or microsatellite metastasis; pTNM = pathological tumor, nodes, and metastasis.

TABLE 25
ECOG Performance Status

| Grade | Description |
|---|---|
| 0 | Normal activity. Fully active, able to carry on all predisease |
| | performance without restriction. |
| 1 | Symptoms, but ambulatory. Restricted in physically strenuous |
| | activity, but ambulatory and able to carry out work of a light |
| | or sedentary nature (e.g., light housework, office work). |
| 2 | In bed <50% of the time. Ambulatory and capable of all self- |
| | care, but unable to carry out any work activities. Up and |
| | about >50% of waking hours. |
| 3 | In bed >50% of the time. Capable of only limited self-care, |
| | confined to bed or chair more than 50% of waking hours. |
| 4 | 100% bedridden. Completely disabled. Cannot carry on any |
| | self-care. Totally confined to bed or chair. |
| 5 | Dead. |

Abbreviations:
ECOG = Eastern Cooperative Oncology Group Oken, M. M., Creech, R. H., Tormey, D. C., Horton, J., Davis, T. E., McFadden, E. T., Carbone, P. P.: Toxicity and Response Criteria of The Eastern Cooperative Oncology Group. Am J Clin Oncol 5:649-655, 1982. The Eastern Cooperative Oncology Group, Robert Comis M.D., Group Chair.

### Discontinuation of Study Intervention and Participant Withdrawal

Discontinuation of study intervention does not represent withdrawal from the study. As certain data on clinical events beyond study intervention discontinuation may be important to the study, they must be collected through the participant's last scheduled follow-up, even if the participant has discontinued study intervention. Therefore, all participants who discontinue study intervention prior to completion of the specified treatment period will continue to participate in the study.

Participants may discontinue study intervention at any time for any reason or be discontinued from the study intervention at the discretion of the investigator should any untoward effect occur. In addition, a participant may be discontinued from study intervention by the investigator or the Sponsor if study intervention is inappropriate, the study plan is violated, or for administrative and/or other safety reasons.

A participant must be discontinued from study intervention but continue to be monitored in the study for any of the following reasons:

The participant or participant's legally acceptable representative requests to discontinue study intervention.
The participant has a medical condition or personal circumstance which, in the opinion of the investigator and/or Sponsor, placed the participant at unnecessary risk from continued administration of study intervention.
The participant has a confirmed positive serum pregnancy test.
Documented disease progression. Note: Participants will be permitted to continue treatment beyond documented RECIST 1.1-defined progression if investigator-assessed clinical stability is observed, and the participant is tolerating study intervention. Treatment beyond progressive disease per iRECIST may be permitted in this study following Sponsor consultation and approval. A description of the adaptations and iRECIST process is provided in Appendix 8, with additional details in the iRECIST publication [Seymour, L., et al 2017].
Any progression or recurrence of any malignancy, or any occurrence of another malignancy, which requires active treatment. Exceptions to secondary malignancy include basal cell carcinoma of the skin, squamous cell carcinoma of the skin, new nonulcerated primary melanoma <1 mm in depth with no nodal involvement, Grade 1 follicular lymphoma or carcinoma in situ (e.g., breast carcinoma, cervical cancer in situ) that have undergone potentially curative therapy. Exceptions should be discussed with the Sponsor prior to continuing therapy.
Recurrent Grade 2 pneumonitis and other AEs that may require treatment discontinuation (Dose Modification).
Completion of approximately 2 years of study intervention.

### Informed Consent

The investigator or medically qualified designee (consistent with local requirements) must obtain documented consent from each potential participant or each participant's legally acceptable representative prior to participating in a clinical study. If there are changes to the participant's status during the study (e.g., health or age of majority requirements), the investigator or medically qualified designee must ensure the appropriate consent is in place.

### Tumor Imaging and Assessment of Disease

The process for image collection and transmission to the CIV can be found in the site imaging manual.

Diagnostic quality CT of the chest, abdomen, pelvis, and neck (if disease present) is required at baseline and at all scheduled follow-up visits. Tumor imaging is strongly preferred to be acquired by CT; the CT portion of PET/CT may be used in place of stand-alone CT if it is of diagnostic quality per the Site Imaging Manual. If a site's standard practice is to follow participants with PET/CT rather than a dedicated CT, these initial images must show progression on prior anti-PD-1/L1 inhibitor, consistent with RECIST 1.1 and iRECIST. There will be no allowance for “worsening of existing disease” by PET if there is NO new lesion. There must be a NEW lesion seen either on PET/CT, or diagnostic quality CT. Refer to the Site Imaging Manual for additional information.

For the abdomen and pelvis, contrast-enhanced MRI may be used when CT with iodinated contrast is contraindicated, or when mandated by local practice. Magnetic resonance imaging is the strongly preferred modality for imaging the brain. The same imaging technique regarding modality, ideally the same scanner, and the use of contrast should be used in a participant throughout this study to optimize the reproducibility of the assessment of existing and new tumor burden and improve the accuracy of the assessment of response or progression based on imaging. Note: for the purposes of assessing tumor imaging, the term “investigator” refers to the local investigator at the site and/or the radiological reviewer at the site or at an offsite facility.

Brain imaging is required for all participants at Screening. Magnetic resonance imaging is preferred; however, CT imaging will be acceptable, if MRI is medically contraindicated.

All scheduled images performed at Screening for all study participants from the sites will be submitted to the CIV to verify the presence of measurable disease by BICR per RECIST 1.1 prior to randomization. All subsequent scheduled images for all study participants from the sites will be submitted to the CIV. In addition, images (including via other modalities) that are obtained at an unscheduled time point to determine disease progression, as well as imaging obtained for other reasons, and captures radiologic progression based on investigator assessment, should also be submitted to the CIV.

### Initial Tumor Imaging

Initial tumor imaging at Screening must be performed within 28 days prior to the date of the first dose of study intervention. If additional time is needed for initial image submission (screening imaging for real-time confirmation of measurable disease per RECIST 1.1 by BICR) and CIV diagnostic quality review (for real-time quality review of prestudy images by BICR), an extension of the screening window of up to 7 days may be approved following mandatory Sponsor consultation. Tumor imaging performed as part of routine clinical management is acceptable for use as Screening tumor imaging if it is of diagnostic quality and performed within 28 days prior to the first dose of study intervention.

Participants in this study with previously treated brain metastases may participate provided they have stable brain metastases, i.e, without evidence of progression by imaging (confirmed by MRI if MRI was used at prior imaging or confirmed by CT imaging if CT used at prior imaging prior to the first dose of study intervention as evidenced by 2 scans at least 4 weeks apart providing stability).

### Tumor Imaging During the Study

The first on-study imaging assessment should be performed at 9 weeks (63 days±7 days) from the date of randomization. Subsequent tumor imaging should be performed every 9 weeks (63 days±7 days) for the first year (until Week 52), after which the imaging interval increases to every 12 weeks (84 days±7 days) for −2 years (until Week 104); and Q24W thereafter or sooner if clinically indicated. Imaging timing should follow calendar days and should not be adjusted for delays in cycle starts. Imaging should continue to be performed until disease progression is identified by the investigator per RECIST 1.1 unless the investigator elects to continue treatment and follow iRECIST, the start of new anticancer treatment, withdrawal of consent, or death, whichever occurs first. All supplemental imaging must be submitted to the CIV.

Per iRECIST, disease progression should be confirmed by the site 4 to 8 weeks after site-assessed first radiologic evidence of progressive disease in clinically stable participants. Participants who have unconfirmed disease progression may continue on treatment at the discretion of the investigator until progression is confirmed by the site. Participants who receive confirmatory imaging do not need to undergo the next scheduled tumor imaging if it is less than 4 weeks later; tumor imaging may resume at the subsequent scheduled imaging time point, if clinically stable. Participants who have confirmed disease progression by iRECIST, as assessed by the site, will discontinue study intervention.

### End of Treatment and Follow Up Tumor Imaging

For participants who discontinue study intervention, tumor imaging should be performed at the time of treatment discontinuation (±4-week window). If previous imaging was obtained within 4 weeks prior to the date of discontinuation, then imaging at treatment discontinuation is not mandatory. For participants who discontinue study intervention due to documented disease progression, this is the final required tumor imaging if the investigator elects not to implement iRECIST.

For participants who discontinue study intervention without documented disease progression, every effort should be made to continue monitoring disease status by tumor imaging using the same imaging schedule used while on treatment (every 9 weeks for the first year [through Week 52] and approximately every 12 weeks after Year 1) until the start of a new anticancer treatment, disease progression, pregnancy, death, withdrawal of consent, or the end of this study, whichever occurs first.

### RECIST 1.1 Assessment of Disease

RECIST 1.1 will be used as the primary measure for assessment of tumor response, date of disease progression, and as a basis for all protocol guidelines related to disease status (e.g., discontinuation of study intervention). Although RECIST 1.1 references a maximum of 5 target lesions in total and 2 per organ, this protocol allows a maximum of 10 target lesions in total and 5 per organ, if clinically relevant to enable a broader sampling of tumor burden.

iRECIST is based on RECIST 1.1 but adapted to account for the unique tumor response seen with immunotherapeutic drugs. iRECIST will be used by the investigator to assess tumor response and progression and make treatment decisions. When clinically stable, participants should not be discontinued until progression is confirmed by the investigator. This allowance to continue treatment despite initial radiologic progressive disease takes into account the observation that some participants can have a transient tumor flare in the first few months after the start of immunotherapy, and then experience subsequent disease response. This data will be captured in the clinical database.

Any participant deemed clinically unstable should be discontinued from study intervention at central verification of site-assessed first radiologic evidence of progressive disease and is not required to have repeat tumor imaging for confirmation of progressive disease by iRECIST.

If the investigator decides to continue treatment, the participant may continue to receive study intervention and the tumor assessment should be repeated 4 to 8 weeks later to confirm progressive disease by iRECIST, per investigator assessment. Images should continue to be sent in to the central imaging vendor for potential retrospective BICR.

If repeat imaging does not confirm progressive disease per iRECIST, as assessed by the investigator, and the participant continues to be clinically stable, study intervention may continue and follow the regular imaging schedule. If progressive disease is confirmed, participants will be discontinued from study intervention.

If a participant has confirmed radiographic progression (iCPD), study intervention should be discontinued; however, if the participant is achieving a clinically meaningful benefit, an exception to continue study intervention may be considered following consultation with the Sponsor.

A summary of imaging and treatment requirements after first radiologic evidence of progression is provided in Table 24.

TABLE 26
Imaging and Treatment After First Radiologic Evidence of Progressive Disease

| | Clinically Stable | | Clinically Unstable | |
|---|---|---|---|---|
| | Imaging | Treatment | Imaging | Treatment |
| First radiologic | Repeat imaging | May continue study | Repeat imaging | Discontinue |
| evidence of | at 4 to 8 weeks | intervention at the | at 4 to 8 weeks | treatment. |
| progressive | to confirm | investigator's | to confirm | |
| disease by | progressive | discretion while | progressive | |
| RECIST 1.1 | disease. | awaiting confirmatory | disease per | |
| | | tumor imaging by | investigator's | |
| | | site by iRECIST. | discretion only. | |
| Repeat tumor | No additional | Discontinue | No additional | Not applicable. |
| imaging | imaging | treatment | imaging required. | |
| confirms PD | required. | (exception is | | |
| (iCPD) by | | possible upon | | |
| iRECIST per | | consultation | | |
| investigator | | with Sponsor). | | |
| assessment. | | | | |
| Repeat tumor | Repeat imaging | Continue study | Repeat imaging | Discontinue |
| imaging shows | at 4 to 8 weeks | intervention at the | at 4 to 8 weeks | treatment. |
| iUPD by | to confirm | investigator's | to confirm | |
| iRECIST per | progressive | discretion. | progressive | |
| investigator | disease. May | | disease per | |
| assessment. | occur at next | | investigator's | |
| | regularly | | discretion only. | |
| | scheduled | | | |
| | imaging visit. | | | |
| Repeat tumor | Continue | Continue study | Continue regularly | May restart study |
| imaging shows | regularly | intervention at the | scheduled imaging | intervention if |
| iSD, iPR, or iCR | scheduled | investigator's | assessments. | condition has |
| by iRECIST per | imaging | discretion | | improved and/or |
| investigator | assessments. | | | clinically stable |
| assessment. | | | | per investigator's |
| | | | | discretion. Next |
| | | | | tumor imaging |
| | | | | should occur |
| | | | | according to the |
| | | | | regular imaging |
| | | | | schedule. |

### Safety Assessment

Safety assessments include the collection of AEs and SAEs, monitoring of vital signs, physical examinations, performance of electrocardiograms (ECGs), a MUGA scan (using technetium-based tracer) or an ECHO at screening to assess LVEF), and pregnancy tests, among others.

### Safety Endpoints

The safety endpoints include AEs, SAEs, and study intervention discontinuation due to AEs. In addition, safety and tolerability will be assessed by clinical review of all relevant parameters including AEs, laboratory tests, and vital signs.

### Adverse Event (AE)

An AE is any untoward medical occurrence in a clinical study participant, temporally associated with the use of study intervention, whether or not considered related to the study intervention.
NOTE: An AE can therefore be any unfavorable and unintended sign (including an abnormal laboratory finding), symptom, or disease (new or exacerbated) temporally associated with the use of a study intervention.

The following are included as AEs:
Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator.
Exacerbation of a chronic or intermittent pre-existing condition including either an increase in frequency and/or intensity of the condition.
New conditions detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.
For all reports of overdose (whether accidental or intentional) with an associated AE, the AE term should reflect the clinical symptoms or abnormal test result. An overdose without any associated clinical symptoms or abnormal laboratory results is reported using the terminology “accidental or intentional overdose without adverse effect.”

The following events do not meet the AE definition for the purpose of this study:
Medical or surgical procedure (e.g., endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen.
Surgery planned prior to informed consent to treat a pre-existing condition that has not worsened.

### Serious Adverse Event (SAE)

If an event is not an AE per the above, then it cannot be an SAE even if serious conditions are met. An SAE is defined as any untoward medical occurrence that, at any dose:
Results in death.
Is life-threatening. The term “life-threatening” in the definition of “serious” refers to an event in which the participant was at risk of death at the time of the event. It does not refer to an event, which hypothetically might have caused death, if it were more severe.
Requires inpatient hospitalization or prolongation of existing hospitalization. Hospitalization is defined as an inpatient admission, regardless of length of stay, even if the hospitalization is a precautionary measure for continued observation. (Note: Hospitalization for an elective procedure to treat a pre-existing condition that has not worsened is not an SAE. A pre-existing condition is a clinical condition that is diagnosed prior to the use of an MSD product and is documented in the participant's medical history.
Results in persistent or significant disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.
Is a congenital anomaly/birth defect. In offspring of participant taking the product regardless of time to diagnosis.
Other important medical events. Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

### Objective Response Rate (ORR)

The ORR is defined as the percentage of participants who achieve a confirmed CR or PR per RECIST 1.1 as assessed by BICR. Participants without follow-up scans will be considered nonresponders. ORR as assessed by investigator per RECIST 1.1 and iRECIST are considered exploratory endpoints.

### Duration of Response (DOR)

For participants who demonstrate confirmed CR or PR per RECIST 1.1 as assessed by BICR, duration of response is defined as the time from the first documented evidence of CR or PR until disease progression or death due to any cause, whichever occurs first.

### Progression-free Survival (PFS)

Progression-free survival is defined as the time from date of randomization/allocation to the first documented progressive disease per RECIST 1.1 by BICR, or death due to any cause, whichever occurs first.

### Overall Survival (OS)

Overall survival is defined as the time from date of randomization/allocation to date of death from any cause.

The study treatments are outlined below in Table 27.

TABLE 27
Study Treatments

| Study | | | Unit | | | Regimen/ |
|---|---|---|---|---|---|---|
| Treatment | Dosage | Dose | Dose | Dosage | Route of | Treatment |
| Name | Formulation | Frequency | Strengths | Level(s) | Administration | Period |
| MK-1308 | Solution for | Every | 50 mg/ml | 25 mg | IV infusion | Day 1 of each |
| | infusion | 6 weeks | | | | 21-day cycle |
| | | (Q6W) | | | | for up to 2 |
| | | | | | | years of |
| | | | | | | treatment |
| Pembrolizu | Solution for | Every | | 400 mg | IV infusion | Day 1 of each |
| mab | infusion | 6 weeks | | | | 21-day cycle |
| | | (Q6W) | | | | for up to 2 |
| | | | | | | years of |
| | | | | | | treatment |
| Lenvatinib | Capsule | Once Daily | 10 mg | 10, 14, | Oral | Daily on a 21- |
| | | (QD) | 4 mg | or 20 | | day cycle for |
| | | | | mg | | up to 2 years of |
| | | | | | | treatment |

* 4 mg capsules provided for successive dose reduction of lenvatinib, if needed

TABLE 28
Investigational Summary
Pembrolizumab + MK-1308 + Lenvatinib (SoA applicable to participants in the Safety Lead-In and Efficacy Phase)

| Study | | | | | | | | | Posttreatment | | | | |
|---|---|---|---|---|---|---|---|---|---|---|---|---|---|
| Period | Treatment Cycle = 21 Days | | | | | | | EOT^{b} | Safety | Follow-Up | | Survival | Notes |
| Visit Timing / | 1 | | | 2 | | | Cycle 3 | At | 30 | Q12W | Q24W | Q12W | All procedures and assessments are to be |
| Cycle Number | | | | | | | and | D/C | Days | | | | performed prior to administration of study |
| | | | | | | | Onwards^{a} | | After | | | | intervention unless otherwise indicated. |
| | | | | | | | | | Last | | | | |
| | | | | | | | | | Dose | | | | |
| Cycle Day | 1 | 8 | 15 | 1 | 8 | 15 | 1 | | | | | | |
| Scheduling | | ±3 | ±3 | ±3 | ±3 | ±3 | ±3 | | +7 | ±7 | ±7 | ±7 | |
| Window (Days) | | | | | | | | | | | | | |

Administrative Procedures

| | | | | | | | | | | | | | |
|---|---|---|---|---|---|---|---|---|---|---|---|---|---|
| Concomitant | X | X | X | X | X | X | X | X | X | X | | | Concomitant medications will be recorded for |
| Medication | | | | | | | | | | | | | 90 days after last dose (or up to 120 days after |
| Review | | | | | | | | | | | | | last dose for SAEs). |
| Randomization/ | X | | | | | | | | | | | | Participants may be randomized/allocated up to |
| Allocation | | | | | | | | | | | | | 3 days prior to first dose of study intervention |
| | | | | | | | | | | | | | and after confirmation of eligibility. |
| | | | | | | | | | | | | | All procedures and assessments on first dose of |
| | | | | | | | | | | | | | study intervention should be performed after |
| | | | | | | | | | | | | | randomization/allocation. |
| Dispensation of | X | | | | | | | X | | | | | Day 1 of every cycle (eg. C1D1, C2D1, C3D1, |
| Pembrolizumab | | | | | | | | | | | | | etc.) |
| Drug Kit | | | | | | | | | | | | | |
| Pembrolizumab | X | | | | | | | X | | | | | Pembrolizumab will be administered Q6W (ie., |
| Administration | | | | | | | | | | | | | every other cycle) for up to 2 years of |
| | | | | | | | | | | | | | treatment. |
| Dispensation of | X | | | | | | | X | | | | | Day 1 of every cycle (eg. C1D1, C2D1, C3D1, |
| MK-1308 drug | | | | | | | | | | | | | etc.) |
| kit | | | | | | | | | | | | | |
| MK-1308 | X | | | | | | | X | | | | | MK-1308 will be administered Q6W (ie, every |
| administration | | | | | | | | | | | | | other cycle) for up to 2 years of treatment. |
| Dispensation of | X | | | X | | | | X | | | | | Day lof every cycle (eg., C1D1, C2D1, C3D1, |
| Lenvatinib drug | | | | | | | | | | | | | etc.) |
| kit | | | | | | | | | | | | | Note: Dose reductions of lenvatinib in the |
| | | | | | | | | | | | | | middle of a cycle will be allowed. |
| Lenvatinib | X | X | X | X | X | X | X | | | | | | Lenvatinib will be administered daily on a 21- |
| Administration | | | | | | | | | | | | | day cycle for up to 2 years of treatment. On |
| | | | | | | | | | | | | | C1D1, C2D1, C3D1, etc, lenvatinib will be |
| | | | | | | | | | | | | | taken 0-4 hours after completion of |
| | | | | | | | | | | | | | pembrolizumab and MK-1308 administration, |
| | | | | | | | | | | | | | if applicable. |
| | | | | | | | | | | | | | Lenvatinib will be taken in the clinic on CID 1, |
| | | | | | | | | | | | | | C3D1, C3D1, etc. |
| | | | | | | | | | | | | | Participants may be allowed to continue |
| | | | | | | | | | | | | | lenvatinib beyond 2 years, at the investigator’s |
| | | | | | | | | | | | | | discretion if there is clinical benefit. |
| Subsequent | | | | | | | | | X | X | X | X | All anticancer therapy will be recorded until |
| Antineoplastic | | | | | | | | | | | | | time of death or termination of survival follow- |
| treatment | | | | | | | | | | | | | up. If a clinic visit is not feasible, follow-up |
| | | | | | | | | | | | | | information may be obtained via telephone or |
| | | | | | | | | | | | | | email. |

^{a}Participants who attain an investigator-determined complete response (CR) and stop study intervention will have 30-day Safety Follow-up
visit and move to the follow-up visits per SoA.
^{b}If EOT visit occurs~30 days from last dose of study intervention, a 30-day Safety Follow-up visit is not required. In this situation, all
procedures required at the 30-day Safety Follow-up visit and EOT are performed once and entered into the EOT visit only. End-of-treatment will
be defined as the date when the participant discontinues all study interventions.
^{c}For participants who D/C study intervention for reasons other than progressive disease, follow-up visits to monitor disease status continue
until progressive disease or initiation of a new anticancer therapy. Participants who D/C study intervention due to progressive disease will
proceed directly to Survival Follow-up.
^{d}a brain MRI must be performed at screening for all participants. For participants with brain metastasis at screening, brain MRI should then be
performed Q9W until Week 52 of study intervention and Q12W thereafter until Week 104; following Week 104, imaging should be performed
every Q24W, or sooner if clinically indicated. Brain CT scan should only be used when MRI is contraindicated. The same imaging technique
regarding modality and the use of contrast should be used in a participant throughout the study to optimize the visualization of existing and new
tumor burden.

### V. SEQUENCE LISTING

The present specification is being filed with a computer readable form (CRF) copy of the Sequence Listing. The CRF entitled 24797-WO-PCT-SEQLIST.txt, which was created on Feb. 18, 2021 and is 40.0 KB in size, is incorporated herein by reference in its entirety.

Table 29 below summarizes all sequences disclosed in the present specification.

TABLE 29
SEQ ID NOS and Corresponding Sequences

| SEQ | | |
|---|---|---|
| ID | | |
| NO | Description | Sequence |
| 1 | CDRH1 | GFTFSDNW |
| 2 | CDRH2 | IRNKPYNYET |
| 3 | CDRH3 | TAQFAY |
| 4 | CDRL1 | ENIYGG |
| 5 | CDRL2 | GAT |
| 6 | CDRL3 | QNVLRSPFT |
| 7 | CDRL3 | QNVLSRHPG |
| 8 | CDRL3 | QNVLSSRPG |
| 9 | VH 8D2/8D2 | EVKLDETGGGLVQPGRPMKLSCVASGFTFSDNWMNW |
| | (RE) | VRQSPEKGLEWLAQIRNKPYNYETYYSDSVKGRFTISR |
| | | DDSKSSVYLQMNNLRGEDMGIYYCTAQFAYWGQGTL |
| | | VTVSA |
| 10 | VL 8D2/8D2 | DIQMTQSPASLSASVGETVTITCGTSENIYGGLNWYQRK |
| | (RE) | QGKSPQLLIFGATNLADGMSSRFSGSGSGRQYSLKISSL |
| | | HPDDVATYYCQNVLRSPFTFGSGTKLEI |
| 11 | VH 8D2H1L1 | EVQLVESGGGLVQPGGSMRLSCAASGFTFSDNWMNW |
| | | VRQAPGKGLEWLAQIRNKPYNYETYYSDSVKGRFTISR |
| | | DDSKNSVYLQMNSLKTEDTGVYYCTAQFAYWGQGTL |
| | | VTVSS |
| 12 | VL 8D2H1L1 | DIQMTQSPSSLSASVGDRVTITCRTSENIYGGLNWYQRK |
| | | QGKSPKLLIYGATNLASGMSSRFSGSGSGTDYTLKISSL |
| | | HPDDVATYYCQNVLRSPFTFGSGTKLEIK |
| 13 | VH 8D2H2L2 | EVQLVESGGGLVQPGGSMRLSCAASGFTFSDNWMNW |
| | | VRQAPGKGLEWLAQIRNKPYNYETYYSASVKGRFTISR |
| | | DDSKNSVYLQMNSLKTEDTGVYYCTAQFAYWGQGTL |
| | | VTVSS |
| 14 | VL | DIQMTQSPSSLSASVGDRVTITCRTSENIYGGLNWYQRK |
| | 8D2H2L2 and | PGKSPKLLIYGATNLASGVSSRFSGSGSGTDYTLTISSLQ |
| | 8D2H2L2 | PEDVATYYCQNVLRSPFTFGSGTKLEIK |
| | VARIANT 1 | |
| 15 | VH | EVQLVESGGGLVQPGGSLRLSCAASGFTFSDNWMNWV |
| | 8D2H2L2 | RQAPGKGLEWLAQIRNKPYNYETYYSASVKGRFTISRD |
| | VARIANT 1 | DSKNSVYLQMNSLKTEDTGVYYCTAQFAYWGQGTLV |
| | | TVSS |
| 16 | VH 8D2H3L3 | EVQLVESGGGLVQPGGSLRLSCAASGFTFSDNWMNWV |
| | | RQAPGKGLEWVAQIRNKPYNYETEYAASVKGRFTISRD |
| | | DSKNSAYLQMNSLKTEDTAVYYCTAQFAYWGQGTLV |
| | | TVSS |
| 17 | VL 8D2H3L3 | DIQMTQSPSSLSASVGDRVTITCRASENIYGGLNWYQQ |
| | | KPGKAPKLLIYGATSLASGVPSRFSGSGSGTDYTLTISSL |
| | | QPEDFATYYCQNVLRSPFTFGSGTKLEIK |
| 18 | VH8D2H2L15 | EVQLVESGGGLVQPGGSMRLSCAASGFTFSDNWMNW |
| | | VRQAPGKGLEWLAQIRNKPYNYETYYSASVKGRFTISR |
| | | DDSKNSVYLQMNSLKTEDTGVYYCTAQFAYWGQGTL |
| | | VTVSS |
| 19 | VL 8D2H2L15 | DIQMTQSPSSLSASVGDRVTITCRTSENIYGGLNWYQRK |
| | | PGKSPKLLIYGATNLASGVSSRFSGSGSGTDYTLTISSLQ |
| | | PEDVATYYCQNVLSRHPGFGSGTKLEIK |
| 20 | VH8D2H2L17 | EVQLVESGGGLVQPGGSMRLSCAASGFTFSDNWMNW |
| | | VRQAPGKGLEWLAQIRNKPYNYETYYSASVKGRFTISR |
| | | DDSKNSVYLQMNSLKTEDTGVYYCTAQFAYWGQGTL |
| | | VTVSS |
| 21 | VL 8D2H2L17 | DIQMTQSPSSLSASVGDRVTITCRTSENIYGGLNWYQRK |
| | | PGKSPKLLIYGATNLASGVSSRFSGSGSGTDYTLTISSLQ |
| | | PED VAT YYCQNVLS SRPGFGSGTKLEIK |
| 22 | Full Heavy | EVQLVESGGGLVQPGGSLRLSCAASGFTFSDNWMNWV |
| | Chain | RQAPGKGLEWLAQIRNKPYNYETYYSASVKGRFTISRD |
| | 8D2H2L2 | DSKNSVYLQMNSLKTEDTGVYYCTAQFAYWGQGTLV |
| | VARIANT 1 | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPC |
| | | PAPELLGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPAPIEKTISKAKGQP |
| | | REPQVYTLPPSRDELTKNQVSLTCLVKGFYPSDIAVEWE |
| | | SNGQPENNYKTTPPVLDSDGSFFLYSKLTVDKSRWQQG |
| | | NVFSCSVMHEALHNHYTQKSLSLSPGK |
| 23 | Full Light | DIQMTQSPSSLSASVGDRVTITCRTSENIYGGLNWYQRK |
| | Chain | PGKSPKLLIYGATNLASGVSSRFSGSGSGTDYTLTISSLQ |
| | 8D2H2L2 | PEDVATYYCQNVLRSPFTFGSGTKLEIKRTVAAPSVFIFP |
| | VARIANT 1 | PSDEQLKSGTASVVCLLNNFYPREAKVQWKVDNALQS |
| | | GNSQESVTEQDSKDSTYSLSSTLTLSKADYEKHKVYAC |
| | | EVTHQGLS SP VTKSFNRGEC |
| 24 | CDRL1 | RASQSVGSSYLA |
| | (Ipilimumab) | |
| 25 | CDRL2 | GAFSRAT |
| | (Ipilimumab) | |
| 26 | CDRL3 | QQYGSSPWT |
| | (Ipilimumab) | |
| 27 | CDRH1 | SYTMH |
| | (Ipilimumab) | |
| 28 | CDRH2 | FISYDGNNKYYADSVKG |
| | (Ipilimumab) | |
| 29 | CDRH3 | TGWLGPFDY |
| | (Ipilimumab) | |
| 30 | Heavy chain | QVQLVESGGGVVQPGRSLRLSCAASGFTFSSYTMHWV |
| | VR | RQAPGKGLEWVTFISYDGNNKYYADSVKGRFTISRDNS |
| | (Ipilimumab) | KNTLYLQMNSLRAEDTAIYYCARTGWLGPFDYWGQGT |
| | | LVTVSS |
| 31 | Light chain VR | EIVLTQSPGTLSLSPGERATLSCRASQSVGSSYLAWYQQ |
| | (Ipilimumab) | KPGQAPRLLIYGAFSRATGIPDRFSGSGSGTDFTLTISRL |
| | | E PEDFAVYYCQQYGSSPWTFGQGTKVEIK |
| 32 | Heavy Chain | Gln Val Gln Leu Val Glu Ser Gly Gly Gly Val Val Gln Pro |
| | (Ipilimumab) | Gly Arg Ser Leu Arg Leu Ser Cys Ala Ala Ser Gly Phe Thr |
| | | Phe Ser Ser Tyr Thr Met His Trp Val Arg Gln Ala Pro Gly Lys |
| | | Gly Leu Glu Trp Val Thr Phe Ile Ser Tyr Asp Gly Asn Asn |
| | | Lys Tyr Tyr Ala Asp Ser Val Lys Gly Arg Phe Thr Ile Ser Arg |
| | | Asp Asn Ser Lys Asn Thr Leu Tyr Leu Gln Met Asn Ser Leu |
| | | Arg Ala Glu Asp Thr Ala Ile Tyr Tyr Cys Ala Arg Thr Gly |
| | | Trp Leu Gly Pro Phe Asp Tyr Trp Gly Gln Gly Thr Leu Val |
| | | Thr Val Ser Ser Ala Ser Thr Lys Gly Pro Ser Val Phe Pro Leu |
| | | Ala Pro Ser Ser Lys Ser Thr Ser Gly Gly Thr Ala Ala Leu Gly |
| | | Cys Leu Val Lys Asp Tyr Phe Pro Glu Pro Val Thr Val Ser |
| | | Trp Asn Ser Gly Ala Leu Thr Ser Gly Val His Thr Phe Pro Ala |
| | | Val Leu Gln Ser Ser Gly Leu Tyr Ser Leu Ser Ser Val Val Thr |
| | | Val Pro Ser Ser Ser Leu Gly Thr Gln Thr Tyr Ile Cys Asn Val |
| | | Asn His Lys Pro Ser Asn Thr Lys Val Asp Lys Arg Val Glu |
| | | Pro Lys Ser Cys Asp Lys Thr His Thr Cys Pro Pro Cys Pro |
| | | Ala Pro Glu Leu Leu Gly Gly Pro Ser Val Phe Leu Phe Pro |
| | | Pro Lys Pro Lys Asp Thr Leu Met Ile Ser Arg Thr Pro Glu Val |
| | | Thr Cys Val Val Val Asp Val Ser His Glu Asp Pro Glu Val |
| | | Lys Phe Asn Trp Tyr Val Asp Gly Val Glu Val His Asn Ala |
| | | Lys Thr Lys Pro Arg Glu Glu Gln Tyr Asn Ser Thr Tyr Arg |
| | | Val Val Ser Val Leu Thr Val Leu His Gln Asp Trp Leu Asn |
| | | Gly Lys Glu Tyr Lys Cys Lys Val Ser Asn Lys Ala Leu Pro |
| | | Ala Pro Ile Glu Lys Thr Ile Ser Lys Ala Lys Gly Gln Pro Arg |
| | | Glu Pro Gln Val Tyr Thr Leu Pro Pro Ser Arg Asp Glu Leu |
| | | Thr Lys Asn Gln Val Ser Leu Thr Cys Leu Val Lys Gly Phe |
| | | Tyr Pro Ser Asp Ile Ala Val Glu Trp Glu Ser Asn Gly Gln Pro |
| | | Glu Asn Asn Tyr Lys Thr Thr Pro Pro Val Leu Asp Ser Asp |
| | | Gly Ser Phe Phe Leu Tyr Ser Lys Leu Thr Val Asp Lys Ser |
| | | Arg Trp Gln Gln Gly Asn Val Phe Ser Cys Ser Val Met His |
| | | Glu Ala Leu His Asn His Tyr Thr Gln Lys Ser Leu Ser Leu |
| | | Ser Pro Gly Lys |
| 33 | Light Chain | Glu Ile Val Leu Thr Gln Ser Pro Gly Thr Leu Ser Leu Ser Pro |
| | (Ipilimumab) | Gly Glu Arg Ala Thr Leu Ser Cys Arg Ala Ser Gln Ser Val |
| | | Gly Ser Ser Tyr Leu Ala Trp Tyr Gln Gln Lys Pro Gly Gln Ala |
| | | Pro Arg Leu Leu Ile Tyr Gly Ala Phe Ser Arg Ala Thr Gly He |
| | | Pro Asp Arg Phe Ser Gly Ser Gly Ser Gly Thr Asp Phe Thr |
| | | Leu Thr Ile Ser Arg Leu Glu Pro Glu Asp Phe Ala Val Tyr Tyr |
| | | Cys Gln Gln Tyr Gly Ser Ser Pro Trp Thr Phe Gly Gln Gly |
| | | Thr Lys Val Glu Ile Lys Arg Thr Val Ala Ala Pro Ser Val Phe |
| | | Ile Phe Pro Pro Ser Asp Glu Gln Leu Lys Ser Gly Thr Ala Ser |
| | | Val Val Cys Leu Leu Asn Asn Phe Tyr Pro Arg Glu Ala Lys |
| | | Val Gln Trp Lys Val Asp Asn Ala Leu Gln Ser Gly Asn Ser |
| | | Gln Glu Ser Val Thr Glu Gln Asp Ser Lys Asp Ser Thr Tyr Ser |
| | | Leu Ser Ser Thr Leu Thr Leu Ser Lys Ala Asp Tyr Glu Lys |
| | | His Lys Val Tyr Ala Cys Glu Val Thr His Gln Gly Leu Ser Ser |
| | | Pro Val Thr Lys Ser Phe Asn Arg Gly Glu Cys |
| 34 | Pembrolizumab, | RASKGVSTSGYSYLH |
| | VL-CDRI | |
| 35 | Pembrolizumab, | LASYLES |
| | VL-CDR2 | |
| 36 | Pembrolizumab, | QHSRDLPLT |
| | VL-CDR3 | |
| 37 | Pembrolizumab, | EIVLTQSPATLSLSPGERATLSCRASKGVSTSGYSYLHW |
| | VL | YQQKPGQAPRLLIYLASYLESGVPARFSGSGSGTDFTLTI |
| | | SSLEPEDFAVYYCQHSRDLPLTFGGGTKVEIK |
| 38 | Pembrolizumab, | EIVLTQSPATLSLSPGERATLSCRASKGVSTSGYSYLHW |
| | lightchain | YQQKPGQAPRLLIYLASYLESGVPARFSGSGSGTDFTLTI |
| | | SSLEPEDFAVYYCQHSRDLPLTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 39 | Pembrolizumab, | NYYMY |
| | VH-CDR1 | |
| 40 | Pembrolizumab, | GINPSNGGTNFNEKFKN |
| | VH-CDR2 | |
| 41 | Pembrolizumab, | RDYRFDMGFDY |
| | VH-CDR3 | |
| 42 | Pembrolizumab, | QVQLVQSGVEVKKPGASVKVSCKASGYTFTNYYMYW |
| | VH | VRQAPGQGLEWMGGINPSNGGTNFNEKFKNRVTLTTD |
| | | SSTTTAYMELKSLQFDDTAVYYCARRDYRFDMGFDYW |
| | | GQGTTVTVSS |
| 43 | Pembrolizumab, | QVQLVQSGVEVKKPGASVKVSCKASGYTFTNYYMYW |
| | heavychain | VRQAPGQGLEWMGGINPSNGGTNFNEKFKNRVTLTTD |
| | | SSTTTAYMELKSLQFDDTAVYYCARRDYRFDMGFDYW |
| | | GQGTTVTVSSASTKGPSVFPLAPCSRSTSESTAALGCLV |
| | | KDYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFLGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |

1. A method of treating cancer, comprising administering to a human patient in need thereof:
(a) a PD-1 antagonist;
(b) a CTLA-4 antagonist; and
(c) 4-[3-chloro-4-(cyclopropylaminocarbonyl)aminophenoxy]-7-methoxy-6-quinolinecarboxamide (lenvatinib) or a pharmaceutically acceptable salt thereof.

2. The method of claim 1, wherein the cancer is selected from the group consisting of bladder cancer, breast cancer, non-small cell lung cancer, colorectal cancer, renal cell carcinoma (RCC), hepatocellular carcinoma, and melanoma.

3. The method of claim 2, wherein the cancer is advanced RCC or metastatic RCC.

4-5. (canceled)

6. The method of claim 2, wherein the cancer is advanced melanoma or metastatic melanoma.

7-8. (canceled)

9. A kit comprising:
(a) a PD-1 antagonist;
(b) a CTLA-4 antagonist; and
(c) 4-[3-chloro-4-(cyclopropylaminocarbonyl)aminophenoxy]-7-methoxy-6-quinolinecarboxamide (lenvatinib) or a pharmaceutically acceptable salt thereof.

10-16. (canceled)

17. The method of claim 1, wherein the PD-1 antagonist is an anti-human PD-1 monoclonal antibody or antigen binding fragment thereof.

18-20. (canceled)

21. The method of claim 1, wherein the CTLA-4 antagonist is an anti-human CTLA4 monoclonal antibody or antigen binding fragment thereof.

22-23. (canceled)

24. The method of claim 17, wherein the anti-human PD-1 monoclonal antibody is pembrolizumab.

25. The method of claim 17, wherein the anti-human PD-1 monoclonal antibody is nivolumab or cemiplimab.

26. (canceled)

27. The method of claim 21, wherein the anti-human CTLA-4 monoclonal antibody comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

28. The method of claim 21, wherein the anti-human CTLA-4 monoclonal antibody comprises a V_{L} region comprising an amino acid sequence as set forth in SEQ ID NO:14, and a V_{H} region comprising an amino acid sequence as set forth in SEQ ID NO:15.

29. The method of claim 21, wherein the anti-human CTLA-4 monoclonal antibody comprises a light chain comprising or consisting of an amino acid sequence as set forth in SEQ ID NO: 23 and a heavy chain comprising or consisting of an amino acid sequence as set forth in SEQ ID NO:22.

30. The method, of claim 1, wherein:
(a) the PD-1 antagonist is pembrolizumab; and
(b) the CTLA-4 antagonist is a monoclonal antibody or antigen binding fragment thereof comprising a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

31. The method of claim 1, wherein:
(a) the PD-1 antagonist is nivolumab or cemiplimab; and
(b) the CTLA-4 antagonist is a monoclonal antibody or antigen binding fragment thereof comprising a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively.

32. (canceled)

33. The method of claim 30 wherein the human patient is administered 200 mg, 240 mg, 400 mg, or 2 mg/kg pembrolizumab, and wherein pembrolizumab is administered once every six weeks.

34. The method of claim 30, wherein the human patient is administered 400 mg pembrolizumab, and wherein pembrolizumab is administered once every six weeks.

35. The method of claim 31, wherein the human patient is administered 240 mg or 3 mg/kg nivolumab once every two weeks, 480 mg nivolumab once every four weeks, or 350 mg cemiplimab once every three weeks.

36. (canceled)

37. The method of claim 33, wherein the human patient is administered from about 1 mg to about 200 mg of the anti-human CTLA-4 antibody, and wherein the anti-human CTLA4 antibody is administered once every six weeks.

38. (canceled)

39. The method of claim 33, wherein the human patient is administered 25 mg of the anti-human CTLA-4 antibody, and wherein the anti-human CTLA4 antibody is administered once every six weeks.

40. The method of claim 33, wherein the human patient is administered 8, 10, 12, 14, 18, 20, or 24 mg lenvatinib or a pharmaceutically acceptable salt thereof, and wherein lenvatinib or the pharmaceutically acceptable salt thereof is administered once daily.

41. A method of treating melanoma, comprising administering to a human patient in need thereof:
(a) 400 mg pembrolizumab;
(b) 25 mg of an anti-human CTLA-4 monoclonal antibody or antigen binding fragment thereof that comprises a V_{L} CDR1, a V_{L} CDR2, and a V_{L} CDR3 comprising amino acid sequences as set forth in SEQ ID NOs: 4, 5, and 6, respectively, and a V_{H} CDR1, a V_{H} CDR2, and a V_{H} CDR3 comprising amino acid sequences as set forth in SEQ ID NOs: 1, 2, and 3, respectively; and
(c) 20 mg lenvatinib.

42. The method of claim 41, wherein each of (a) and (b) is administered once every six weeks, and wherein (c) is administered once daily.

43. (canceled)

44. The method of claim 41, wherein (a) and (b) are administered on the same day, and wherein (a) and (b) are administered sequentially or concurrently.

45. The method of claim 1, wherein the pharmaceutically acceptable salt thereof is lenvatinib mesylate.


SEQUENCE LISTING (43 sequences)

SEQ ID NO: 1 - protein, 8 aa, Artificial Sequence
  Monoclonal antibody CDRH1
GFTFSDNW

SEQ ID NO: 2 - protein, 10 aa, Artificial Sequence
  Monoclonal antibody CDRH2
IRNKPYNYET

SEQ ID NO: 3 - protein, 6 aa, Artificial Sequence
  Monoclonal antibody CDRH3
TAQFAY

SEQ ID NO: 4 - protein, 6 aa, Artificial Sequence
  Monoclonal antibody CDRL1
ENIYGG

SEQ ID NO: 5 - protein, 3 aa, Artificial Sequence
  Monoclonal antibody CDRL2
GAT

SEQ ID NO: 6 - protein, 9 aa, Artificial Sequence
  Monoclonal antibody CDRL3
QNVLRSPFT

SEQ ID NO: 7 - protein, 9 aa, Artificial Sequence
  Monoclonal antibody CDRL3
QNVLSRHPG

SEQ ID NO: 8 - protein, 9 aa, Artificial Sequence
  Monoclonal antibody CDRL3
QNVLSSRPG

SEQ ID NO: 9 - protein, 115 aa, Artificial Sequence
  8D2/8D2 (RE) VH
EVKLDETGGGLVQPGRPMKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 10 - protein, 106 aa, Artificial Sequence
  8D2/8D2 (RE) VL
DIQMTQSPASLSASVGETVTITCGTSENIYGGLNWYQRKQGKSPQLLIFGATNLADGMSSRFSGSGSGRQYSLKISSLHPDDVATYYCQNVLRSPFTFGSGTKLEI

SEQ ID NO: 11 - protein, 115 aa, Artificial Sequence
  8D2H1L1 VH
EVQLVESGGGLVQPGGSMRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 12 - protein, 107 aa, Artificial Sequence
  8D2H1L1 VL
DIQMTQSPSSLSASVGDRVTITCRTSENIYGGLNWYQRKQGKSPKLLIYGATNLASGMSSRFSGSGSGTDYTLKISSLHPDDVATYYCQNVLRSPFTFGSGTKLEIK

SEQ ID NO: 13 - protein, 115 aa, Artificial Sequence
  8D2H2L2 VH
EVQLVESGGGLVQPGGSMRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 14 - protein, 107 aa, Artificial Sequence
  8D2H2L2 VL
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIK

SEQ ID NO: 15 - protein, 115 aa, Artificial Sequence
  8D2H2L2 VH - VARIANT 1
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 16 - protein, 115 aa, Artificial Sequence
  8D3H3L3 VL
EVQLVESGGGLVQPGGSLRLSCAASGFTFSDNWMNWVRQAPGKGLEWVAQIRNKPYNYETEYAASVKGRFTISRDDSKNSAYLQMNSLKTEDTAVYYCTAQFAYWGQGTLVTVSS

SEQ ID NO: 17 - protein, 107 aa, Artificial Sequence
  8D3H3L3 VL
DIQMTQSPSSLSASVGDRVTITCRASENIYGGLNWYQQKPGKAPKLLIYGATSLASGVPSRFSGSGSGTDYTLTISSLQPEDFATYYCQNVLRSPFTFGSGTKLEIK

SEQ ID NO: 18 - protein, 115 aa, Artificial Sequence; identical to SEQ ID NO: 13
  8D2H2L15 VH

SEQ ID NO: 19 - protein, 107 aa, Artificial Sequence
  8D2H2L15 VL
DIQMTQSPSSLSASVGDRVTITCRTSENIYGGLNWYQRKPGKSPKLLIYGATNLASGVSSRFSGSGSGTDYTLTISSLQPEDVATYYCQNVLSRHPGFGSGTKLEIK

SEQ ID NO: 20 - protein, 115 aa, Artificial Sequence; identical to SEQ ID NO: 13
  8D2H2L17 VH

SEQ ID NO: 21 - protein, 107 aa, Artificial Sequence
  8D2H2L17 VL
DIQMTQSPSSLSASVGDRVTITCRTSENIYGGLNWYQRKPGKSPKLLIYGATNLASGVSSRFSGSGSGTDYTLTISSLQPEDVATYYCQNVLSSRPGFGSGTKLEIK

SEQ ID NO: 22 - protein, 445 aa, Artificial Sequence
  8D2H2L2 Full Heavy Chain - VARIANT 1
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, Artificial Sequence
  8D2H2L2 Full Light Chain - VARIANT 1
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 12 aa, Artificial Sequence
  Light and heavy chain Ipilimumab - CDRL1
RASQSVGSSYLA

SEQ ID NO: 25 - protein, 7 aa, Artificial Sequence
  Light and heavy chain Ipilimumab - CDRL2
GAFSRAT

SEQ ID NO: 26 - protein, 9 aa, Artificial Sequence
  Light and heavy chain Ipilimumab - CDRL3
QQYGSSPWT

SEQ ID NO: 27 - protein, 5 aa, Artificial Sequence
  Light and heavy chain Ipilimumab - CDRH1
SYTMH

SEQ ID NO: 28 - protein, 17 aa, Artificial Sequence
  Light and heavy chain Ipilimumab - CDRH2
FISYDGNNKYYADSVKG

SEQ ID NO: 29 - protein, 9 aa, Artificial Sequence
  Light and heavy chain Ipilimumab - CDRH3
TGWLGPFDY

SEQ ID NO: 30 - protein, 118 aa, Artificial Sequence
  Heavy chain VR - Ipilimumab
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 31 - protein, 108 aa, Artificial Sequence
  Light chain VR - Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 32 - protein, 448 aa, Artificial Sequence
  Mature heavy chain and the mature  light chain of Ipilimumab
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 215 aa, Artificial Sequence
  Mature heavy chain and the mature  light chain of Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 15 aa, Artificial Sequence
  Pembrolizumab VL-CDR1
RASKGVSTSGYSYLH

SEQ ID NO: 35 - protein, 7 aa, Artificial Sequence
  Pembrolizumab, VL-CDR2
LASYLES

SEQ ID NO: 36 - protein, 9 aa, Artificial Sequence
  Pembrolizumab VL-CDR3
QHSRDLPLT

SEQ ID NO: 37 - protein, 111 aa, Artificial Sequence [sequence omitted]

SEQ ID NO: 38 - protein, 218 aa, Artificial Sequence
  Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 5 aa, Artificial Sequence
  Pembrolizumab VH-CDR1
NYYMY

SEQ ID NO: 40 - protein, 17 aa, Artificial Sequence
  Pembrolizumab VH-CDR2
GINPSNGGTNFNEKFKN

SEQ ID NO: 41 - protein, 11 aa, Artificial Sequence
  Pembrolizumab VH-CDR3
RDYRFDMGFDY

SEQ ID NO: 42 - protein, 120 aa, Artificial Sequence [sequence omitted]

SEQ ID NO: 43 - protein, 447 aa, Artificial Sequence
  Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK